(19) United States
(12) Patent Application Publication
(10) Pub. No.: US 2009/0082329 A1
(43) Pub. Date: Mar. 26, 2009

(54) Novel Sulphur-Containing Cyclic Urea Derivatives, Preparation Thereof and Pharmaceutical Use Thereof as Kinase Inhibitors

(75) Inventors: Frank HALLEY, Chaville (FR); Youssef EL-AHMAD, Creteil (FR); Victor CERTAL, Draweil (FR); Corinne VENOT, Paris (FR); Anne DAGALLIER, Paris (FR); Hartmut STROBEL, Liederbach (DE); Kurt RITTER, Frankfurt (DE); Sven RUF, Mainz (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: AVENTIS PHARMA S.A., Antony (FR)

(21) Appl. No.: 12/173,191

(22) Filed: Jul. 15, 2008

Related U.S. Application Data
(63) Continuation of application No. PCT/FR2007/000080, filed on Jan. 17, 2007.

(30) Foreign Application Priority Data
Jan. 23, 2006 (FR) 0600566

(51) Int. Cl. A61K 31/4545; C07D 401/06; C07D 401/14; C07D 403/06; A61K 31/506; A61K 31/496; A61P 3/00; A61P 37/00; A61P 11/00; A61P 35/00; A61P 9/00; C12N 9/99; A61K 31/5377; C07D 403/14; C07D 413/06; A61K 31/4439

(52) U.S. Cl. 514/210.18; 546/274.4; 546/194; 544/331; 544/322; 544/139; 544/370; 544/238; 514/341; 514/318; 514/275; 514/235.8; 514/254.05; 435/184

(57) ABSTRACT

The disclosure relates to compounds of formula (I):

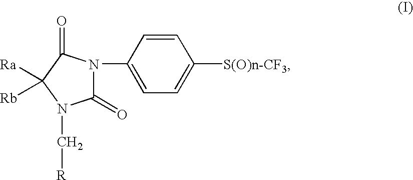

wherein Ra, Rb, R, and n are as defined in the disclosure, to pharmaceutical compositions comprising said compounds, and to processes for making and methods of using the same.

The present invention relates to novel sulfated cyclic urea derivatives, to a process for preparing them, to their use as medicaments, to pharmaceutical compositions containing them and to the pharmaceutical use of such derivatives for preventing and treating complaints that may be modulated by inhibiting the activity of protein kinases.

The present invention relates to novel cyclic urea derivatives that have inhibitory effects on protein kinases.

The products of the present invention may thus be used especially for preventing or treating complaints capable of being modulated by inhibiting the activity of protein kinases.

The inhibition and regulation of protein kinases especially constitute a powerful new mechanism of action for treating a large number of solid or liquid tumours.

Such complaints that the products of the present patent application can treat are thus most particularly solid or liquid tumours.

Such protein kinases belong especially to the following group: EGFR, Fak, FLK-1, FGFR1, FGFR2, FGFR3, FGFR4, FGFR5, flt-1, IGF-1R, KDR, PLK, PDGFR, tie2, VEGFR, AKT, Raf.

The protein kinase IGF1-R (Insulin Growth Factor-1 Receptor) is particularly indicated.

The present invention thus relates particularly to novel inhibitors of the IGF-1R receptor that may be used for oncology treatments.

Cancer remains a disease for which the existing treatments are clearly insufficient. Certain protein kinases, especially including IGF-1R (Insulin Growth Factor 1 Receptor), play an important role in many cancers. The inhibition of such protein kinases is potentially important in the chemotherapy of cancers, especially for suppressing the growth or survival of tumours. The present invention thus relates to the identification of novel products that inhibit such protein kinases.

Protein kinases participate in signalling events that control the activation, growth and differentiation of cells in response either to extracellular mediators or to changes in the environment. In general, these kinases belong to two groups: those that preferentially phosphorylate serine and/or threonine residues and those that preferentially phosphorylate tyrosine residues [S. K. Hanks and T. Hunter, FASEB. J., 1995, 9, pages 576-596]. The serine/threonine kinases are, for example, the isoforms of the protein kinases C [A. C. Newton, J. Biol. Chem., 1995, 270, pages 28495-28498] and a group of cycline-dependent kinases, for instance cdc2 [J. Pines, Trends in Biochemical Sciences, 1995, 18, pages 195-197]. Tyrosine kinases comprise growth factor receptors, for instance the epidermal growth factor (EGF) receptor [S. Iwashita and M. Kobayashi, Cellular Signalling, 1992, 4, pages 123-132], and cytosol kinases, for instance p56tck, p59fYn and ZAP-70 and the kinases csk [C. Chan et. al., Ann. Rev. Immunol., 1994, 12, pages 555-592].

Abnormally high levels of kinase protein activity have been implicated in many diseases, resulting from abnormal cellular functions. This may arise either directly or indirectly from a dysfunction in the mechanisms for controlling the kinase activity, linked, for example, to a mutation, an overexpression or an inappropriate activation of the enzyme, or an over- or underproduction of cytokines or of growth factors, also involved in the transduction of the signals upstream or downstream of the kinases. In all these cases, a selective inhibition of the action of the kinases offers hope of a beneficial effect.

The type 1 receptor for the insulin-like growth factor (IGF-I-R) is a transmembrane receptor with tyrosine kinase activity, which binds firstly to IGFI, but also to IGFII and to insulin with lower affinity. The binding of IGF1 to its receptor results in oligomerization of the receptor, the activation of tyrosine kinase, intermolecular autophosphorylation and the phosphorylation of cell substrates (main substrates: IRS1 and Shc). The receptor activated by its ligand induces mitogenic activity in normal cells. However, IGF-I-R plays an important role in “abnormal” growth.

Several clinical reports underline the important role of the IGF-I route in the development of human cancers:

IGF-I-R is often found overexpressed in many types of tumour (breast, colon, lung, sarcoma, prostate, multiple myeloma) and its presence is often associated with a more aggressive phenotype.

High concentrations of circulating IGF1 are strongly correlated with a risk of prostate cancer, lung cancer and breast cancer.

Furthermore, it has been widely documented that IGF-I-R is necessary for establishing and maintaining the transformed phenotype in vitro as in vivo [Baserga R, Exp. Cell. Res., 1999, 253, pages 1-6]. The kinase activity of IGF-I-R is essential for the transformation activity of several oncogenes: EGFR, PDGFR, the large T antigen of the SV40 virus, activated Ras, Raf, and v-Src. The expression of IGF-I-R in normal fibroblasts induces a neoplastic phenotype, which may then result in the formation of a tumour in vivo. The expression of IGF-I-R plays an important role in substrate-independent growth. IGF-I-R has also been shown to be a protector in chemotherapy-induced and radiation-induced apoptosis, and cytokine-induced apoptosis. Furthermore, the inhibition of endogenous IGF-I-R with a negative dominant, the formation of a triple helix or the expression of an antisense sequence brings about suppression of the transforming activity in vitro and reduction of tumour growth in animal models.

Among the kinases for which a modulation of the activity is desired, FAK (Focal Adhesion Kinase) is also a preferred kinase.

FAK is a cytoplasmic tyrosine kinase that plays an important role in transducing the signal transmitted by the integrins, a family of heterodimeric receptors of cellular adhesion. FAK and the integrins are colocalized in perimembrane structures known as adhesion plaques. It has been shown in many cell types that the activation of FAK and its phosphorylation on tyrosine residues and in particular its autophosphorylation on tyrosine 397 were dependent on the binding of the integrins to their extracellular ligands and thus induced during cellular adhesion [Kornberg L, et al. J. Biol. Chem. 267(33): 23439-442 (1992)]. The autophosphorylation on tyrosine 397 of FAK represents a binding site for another tyrosine kinase, Src, via its SH2 domain [Schaller et al. Mol. Cell. Biol. 14: 1680-1688 1994; Xing et al. Mol. Cell. Biol. 5: 413-421 1994]. Src can then phosphorylate FAK on tyrosine 925, thus recruiting the adapter protein Grb2 and inducing in certain cells activation of the ras and MAP kinase pathway involved in controlling cellular proliferation [Schlaepfer et al. Nature; 372: 786-791 1994; Schlaepfer et al. Prog. Biophys. Mol. Biol. 71: 435-478 1999; Schlaepfer and Hunter, J. Biol. Chem. 272: 13189-13195 1997].

The activation of FAK can thus induce the jun NH2-terminal kinase (JNK) signalling pathway and result in the progression of the cells to the G1 phase of the cellular cycle [Oktay et al., J. Cell. Biol. 145: 1461-1469 1999]. Phosphatidylinositol-3—OH kinase (PI3-kinase) also binds to FAK on tyrosine 397 and this interaction might be necessary for the activation of PI3-kinase [Chen and Guan, Proc. Nat. Acad. Sci. USA. 91: 10148-10152 1994; Ling et al. J. Cell. Biochem. 73: 533-544 1999]. The FAK/Src complex phosphorylates various substrates, for instance paxillin and p130CAS in fibroblasts [Vuori et al. Mol. Cell. Biol. 16: 2606-2613 1996].

The results of numerous studies support the hypothesis that FAK inhibitors might be useful in treating cancer. Studies have suggested that FAK might play an important role in in vitro cell proliferation and/or survival. For example, in CHO cells, certain authors have demonstrated that the overexpression of p125FAK induces an acceleration of the G1 to S transition, suggesting that p125FAK promotes cellular proliferation [Zhao J.-H et al. J. Cell Biol. 143: 1997-2008 1998]. Other authors have shown that tumour cells treated with FAK antisense oligonucleotides lose their adhesion and go into apoptosis (Xu et al, Cell Growth Differ. 4: 413-418 1996). It has also been demonstrated that FAK promotes the migration of cells in vitro. Thus, fibroblasts that are deficient for the expression of FAK (“knockout” mice for FAK) show a rounded morphology and deficiencies in cell migration in response to chemotactic signals, and these defects are suppressed by re-expression of FAK [DJ. Sieg et al., J. Cell Science. 112: 2677-91 1999]. The overexpression of the C-terminal domain of FAK (FRNK) blocks the stretching of adherent cells and reduces cellular migration in vitro [Richardson A. and Parsons J. T. Nature. 380: 538-540 1996]. The overexpression of FAK in CHO or COS cells or in human astrocytoma cells promotes migration of the cells. The involvement of FAK in promoting the proliferation and migration of cells in numerous cell types in vitro suggests the potential role of FAK in neoplastic processes. A recent study has effectively demonstrated the increase in the proliferation of tumour cells in vivo after induction of the expression of FAK in human astrocytoma cells [Cary L. A. et al. J. Cell Sci. 109: 1787-94 1996; Wang D et al. J. Cell Sci. 113: 4221-4230 2000]. Furthermore, immunohistochemical studies on human biopsies have demonstrated that FAK is overexpressed in prostate cancer, breast cancer, thyroid cancer, cancer of the colon, melanoma, brain cancer and lung cancer, the level of expression of FAK being directly correlated to the tumours having the most aggressive phenotype [Weiner T M, et al. Lancet. 342 (8878): 1024-1025 1993; Owens et al. Cancer Research. 55: 2752-2755 1995; Maung K. et al. Oncogene 18: 6824-6828 1999; Wang D et al. J. Cell Sci. 113: 4221-4230 2000].

Protein kinase AKT (also known as PKB) and phosphoinositide 3-kinase (PI3K) are involved in a cell signalling pathway that transmits signals from growth factors activating membrane receptors.

This transduction pathway is involved in numerous cellular functions: regulation of apoptosis, control of transcription and translation, glucose metabolism, angiogenesis and mitochondrial integrity. First identified as an important component of insulin-dependent signalling pathways regulating metabolic responses, serine/threonine kinase AKT was then identified as a mediator playing a key role in survival induced with growth factors. It has been shown that AKT can inhibit death by apoptosis induced by various stimuli, in a certain number of cell types and tumour cells. In accordance with these findings, it has been shown that AKT can, by phosphorylation of given serine residues, inactivate BAD, GSK3β, caspase-9, and Forkhead transcription factor, and can activate IKKalpha and e-NOS. It is interesting to note that the protein BAD is found hyper-phosphorylated in 11 human tumour cell lines out of 41 studied. Furthermore, it has been shown that hypoxia modulates the induction of VEGF in cells transformed with Ha-ras by activating the PI3K/AKT pathway and by involving the binding sequence of the HIF-1 (hypoxia inducible factor-1) transcription factor known as HRE for “hypoxy-responsive element”.

AKT plays a very important role in cancer pathologies. The amplification and/or overexpression of AKT has been reported in many human tumours, for instance gastric carcinoma (amplification of AKT1), ovary carcinoma, breast carcinoma or pancreatic carcinoma (amplification and overexpression of AKT2) and breast carcinomas deficient in oestrogen receptors, and also androgen-independent prostate carcinomas (overexpression of AKT3). Furthermore, AKT is constitutively activated in all the PTEN (−/−) tumours, the PTEN phosphatase being deleted or inactivated by mutations in many types of tumours, for instance carcinomas of the ovary, of the prostate, of the endometrium, glioblastomas and melanomas. AKT is also involved in the oncogenic activation of bcr-abl (references: Khawaja A., Nature 1999, 401, 33-34; Cardone et al. Nature 1998, 282, 1318-1321; Kitada S. et al., Am J Pathol 1998 January; 152(1): 51-61; Mazure N M et al. Blood 1997, 90, 3322-3331; Zhong H. et al. Cancer Res. 2000, 60, 1541-1545).

One subject of the present invention is thus the products of general formula (I):

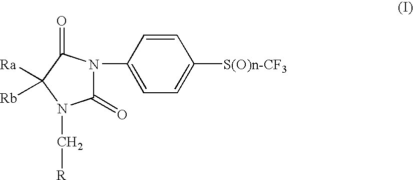

in which:
n represents the integer 0 or 2
Ra and Rb represent CH3 or form, together with the carbon atom to which they are attached, a cycloalkyl radical,
R represents a pyridyl or pyrimidinyl radical substituted with a radical NR1R2,
NR1R2 being such that:
one from among R1 and R2 represents a hydrogen atom or an alkyl radical, and the other from among R1 and R2 is chosen from a hydrogen atom and alkyl radicals optionally substituted with a radical chosen from hydroxyl, alkoxy, aziridyl, azetidinyl, pyrrolidinyl, piperidyl, morpholinyl, and piperazinyl, which is itself optionally substituted on its second nitrogen atom with an alkyl radical; optionally substituted cycloalkyl, heterocycloalkyl, aryl and heteroaryl radicals; and the radical CO—R3 with R3 chosen from NR4R5 and optionally substituted alkoxy, heterocycloalkyl, aryl, aryloxy and heteroaryl radicals;
R4 and R5, which may be identical to or different from R1 and R2, are such that:
either one from among R4 and R5 represents a hydrogen atom or an alkyl radical, and the other from among R4 and R5 is chosen from a hydrogen atom and alkyl radicals optionally substituted with a radical chosen from hydroxyl, alkoxy, aziridyl, azetidinyl, pyrrolidinyl, piperidyl, morpholinyl, and piperazinyl, which is itself optionally substituted on its second nitrogen atom with an alkyl radical; optionally substituted cycloalkyl, heterocycloalkyl, aryl and heteroaryl radicals;
or R4 and R5 form, with the nitrogen atom to which they are attached, a cyclic amine optionally containing another heteroatom chosen from N and O, which is optionally substituted,
all the above aryl, phenyl, aryloxy and heteroaryl radicals, and also the cyclic amine NR4R5, being optionally substituted with one to three radicals, which may be identical or different, chosen from halogen atoms and alkyl, phenyl, NH2, NHAlk, N(Alk)2, CO—NHAlk and CO—N(Alk)2 radicals; the said products of formula (I) being in any possible racemic, enantiomeric or diastereoisomeric isomer form, and also the addition salts with mineral and organic acids or with mineral and organic bases of the said products of formula (I).

It may be noted that when Ra and Rb, together with the carbon atom to which they are attached, form a cycloalkyl radical, this radical is especially cyclopropyl.

A subject of the present invention is thus the products of formula (I) as defined above:

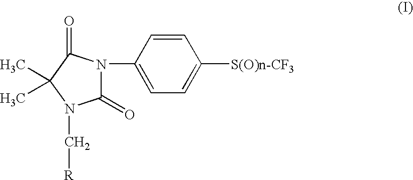

in which:
n represents the integer 0 or 2
Ra and Rb represent CH3,
R represents a pyridyl or pyrimidinyl radical substituted with
a radical NR1R2,
NR1R2 being such that:
one from among R1 and R2 represents a hydrogen atom or an alkyl radical, and the other from among R1 and R2 is chosen from a hydrogen atom and alkyl radicals optionally substituted with a radical chosen from hydroxyl, alkoxy, aziridyl, azetidinyl, pyrrolidinyl, piperidyl, morpholinyl, or piperazinyl, which is itself optionally substituted on its second nitrogen atom with an alkyl radical; optionally substituted cycloalkyl, heterocycloalkyl, phenyl, pyrimidinyl and pyridyl radicals; and the radical CO—R3 with R3 chosen from NR4R5 and optionally substituted alkoxy, piperidyl, phenyl and phenoxy radicals;
R4 and R5, which may be identical to or different from R1 and R2, are such that:
either one from among R4 and R5 represents a hydrogen atom or an alkyl radical, and the other from among R4 and R5 is chosen from a hydrogen atom and alkyl radicals optionally substituted with a radical chosen from hydroxyl, alkoxy, aziridyl, azetidinyl, pyrrolidinyl, piperidyl, morpholinyl, or piperazinyl, which is itself optionally substituted on its second nitrogen atom with an alkyl radical; optionally substituted cycloalkyl, heterocycloalkyl, phenyl, pyrimidinyl and pyridyl radicals; or R4 and R5 form, with the nitrogen atom to which they are attached, a cyclic amine optionally containing another heteroatom chosen from N and O, which is optionally substituted,
all the above phenyl, pyrimidinyl and pyridyl radicals being optionally substituted with one to three radicals, which may be identical or different, chosen from halogen atoms and alkyl, phenyl, NH2, NHAlk, N(Alk)2, CO—NHAlk and CO—N(Alk)2 radicals; the said products of formula (I) being in any possible racemic, enantiomeric and diastereoisomeric isomer form, and also the addition salts with mineral and organic acids or with mineral and organic bases of the said products of formula (I).

In the products of formula (I) and hereinbelow, the terms indicated have the following meanings:
the term “Hal”, “Halo” or halogen denotes fluorine, chlorine, bromine or iodine atoms, and preferably fluorine and chlorine,
the term “alkyl” or “alk” denotes a linear or branched radical containing not more than 12 carbon atoms, chosen from methyl, ethyl, propyl, isopropyl, butyl, isobutyl, sec-butyl, tert-butyl, pentyl, isopentyl, sec-pentyl, tert-pentyl, neopentyl, hexyl, isohexyl, sec-hexyl, tert-hexyl, heptyl, octyl, nonyl, decyl, undecyl and dodecyl radicals, and also the linear or branched positional isomers thereof.

Mention is made more particularly of alkyl radicals containing not more than 6 carbon atoms, and especially methyl, ethyl, propyl, isopropyl, n-butyl, isobutyl, tert-butyl, linear or branched pentyl and linear or branched hexyl radicals.
the term “alkoxy radical” denotes a linear or branched radical containing not more than 12 carbon atoms and preferably 6 carbon atoms chosen, for example, from methoxy, ethoxy, propoxy, isopropoxy, linear, secondary or tertiary butoxy, pentoxy, hexoxy and heptoxy radicals, and also the linear or branched positional isomers thereof,
the term “cycloalkyl radical” denotes a 3- to 10-membered monocyclic or bicyclic carbocyclic radical and especially denotes cyclopropyl, cyclobutyl, cyclopentyl and cyclohexyl radicals,
the term “acyl radical” or —CO-r denotes a linear or branched radical containing not more than 12 carbon atoms, in which the radical r represents a hydrogen atom or an alkyl, cycloalkyl, cycloalkenyl, cycloalkyl, heterocycloalkyl or aryl radical, these radicals having the values indicated above and being optionally substituted as indicated: examples that are mentioned include the formyl, acetyl, propionyl, butyryl or benzoyl radical, or alternatively valeryl, hexanoyl, acryloyl, crotonoyl or carbamoyl. It is noted that the radical CO—R₃ can especially take the values defined above for —CO-r,
the term “aryl radical” denotes unsaturated monocyclic radicals or unsaturated radicals consisting of fused carbocyclic rings. Examples of such aryl radicals that may be mentioned include phenyl or naphthyl radicals.

Mention is made more particularly of the phenyl radical.

The aryloxy radical denotes a radical —O-aryl in which the aryl radical has the meaning indicated above.

The term “heterocycloalkyl radical” denotes a saturated carbocyclic radical which is not more than 7-membered, interrupted with one or more heteroatoms, which may be identical or different, chosen from oxygen, nitrogen and sulfur atoms: heterocycloalkyl radicals that may especially be mentioned include dioxolane, dioxane, dithiolan, thiooxolane, thioxane, oxiranyl, oxylanyl, dioxolanyl, piperazinyl, piperidyl, pyrrolidinyl, imidazolidinyl, pyrazolidinyl, morpholinyl, or tetrahydrofuryl, tetrahydrothienyl, chromanyl, dihydrobenzofuryl, indolinyl, piperidyl, perhydropyranyl, pyrindolinyl, tetrahydroquinolyl, tetrahydroisoquinolyl and thiazolidinyl radicals, all these radicals being optionally substituted.

Among the heterocycloalkyl radicals that may especially be mentioned are optionally substituted piperazinyl, optionally substituted piperidyl, optionally substituted pyrrolidinyl, imidazolidinyl, pyrazolidinyl, morpholinyl and thiazolidinyl radicals.

The term “heteroaryl radical” denotes a partially or totally unsaturated carbocyclic radical which is not more than 7-membered, interrupted with one or more heteroatoms, which may be identical or different, chosen from oxygen, nitrogen and sulfur atoms; among the 5-membered heteroaryl radicals that may be mentioned are furyl radicals such as 2-furyl, thienyl radicals such as 2-thienyl and 3-thienyl, and pyrrolyl, diazolyl, thiazolyl, thiadiazolyl, thiatriazolyl, isothiazolyl, oxazolyl, oxadiazolyl, 3- or 4-isoxazolyl, imidazolyl, pyrazolyl and isoxazolyl radicals. Among the 6-membered heteroaryl radicals that may especially be mentioned are pyridyl radicals such as 2-pyridyl, 3-pyridyl and 4-pyridyl, and pyrimidyl, pyrimidinyl, pyridazinyl, pyrazinyl and tetrazolyl radicals.
as fused heteroaryl radicals containing at least one hetero atom chosen from sulfur, nitrogen and oxygen, examples that may be mentioned include benzothienyl such as 3-benzothienyl, benzofuryl, benzofuryl, benzopyrrolyl, benzimidazolyl, benzoxazolyl, thionaphthyl, indolyl, purinyl, quinolyl, isoquinolyl and naphthyridinyl.

Among the fused heteroaryl radicals that may be mentioned more particularly are benzothienyl, benzofuryl, indolyl, quinolyl, benzimidazolyl, benzothiazolyl, furyl, imidazolyl, indolizinyl, isoxazolyl, isoquinolyl, isothiazolyl, oxadiazolyl, pyrazinyl, pyridazinyl, pyrazolyl, pyridyl, pyrimidinyl, pyrrolyl, quinazolinyl, 1,3,4-thiadiazolyl, thiazolyl and thienyl radicals and triazolyl groups, these radicals optionally being substituted as indicated for the heteroaryl radicals.

The term “patient” denotes human beings, but also other mammals.

The term “prodrug” denotes a product that may be converted in vivo via metabolic mechanisms (such as hydrolysis) into a product of formula (I). For example, an ester of a product of formula (I) containing a hydroxyl group may be converted by hydrolysis in vivo into its parent molecule. Alternatively, an ester of a product of formula (I) containing a carboxyl group may be converted by in vivo hydrolysis into its parent molecule.

Examples of esters of the products of formula (I) containing a hydroxyl group that may be mentioned include the acetates, citrates, lactates, tartrates, malonates, oxalates, salicylates, propionates, succinates, fumarates, maleates, methylenebis-β-hydroxynaphthoates, gentisates, isethionates, di-p-tolyltartrates, methanesulfonates, ethanesulfonates, benzenesulfonates, p-toluenesulfonates, cyclohexylsulfamates and quinates.

Esters of products of formula (I) that are particularly useful, containing a hydroxyl group, may be prepared from acid residues such as those described by Bundgaard et al., J. Med. Chem., 1989, 32, pp. 2503-2507: these esters especially include substituted (aminomethyl)benzoates, dialkylaminomethylbenzoates in which the two alkyl groups may be linked together or may be interrupted with an oxygen atom or with an optionally substituted nitrogen atom, i.e. an alkylated nitrogen atom, or alternatively (morpholinomethyl)benzoates, e.g. 3- or 4-(morpholinomethyl)benzoates, and (4-alkylpiperazin-1-yl)benzoates, e.g. 3- or 4-(4-alkylpiperazin-1-yl)benzoates.

The carboxyl radical(s) of the products of formula (I) may be salified or esterified with various groups known to those skilled in the art, among which nonlimiting examples that may be mentioned include the following compounds:
among the salification compounds, mineral bases such as, for example, one equivalent of sodium, potassium, lithium, calcium, magnesium or ammonium, or organic bases such as, for example, methylamine, propylamine, trimethylamine, diethylamine, triethylamine, N,N-dimethylethanolamine, tris(hydroxymethyl)aminomethane, ethanolamine, pyridine, picoline, dicyclohexylamine, morpholine, benzylamine, procaine, lysine, arginine, histidine or N-methylglucamine,
among the esterification compounds, alkyl radicals to form alkoxycarbonyl groups such as, for example, methoxycarbonyl, ethoxycarbonyl, tert-butoxycarbonyl or benzyloxycarbonyl, these alkyl radicals possibly being substituted with radicals chosen, for example, from halogen atoms and hydroxyl, alkoxy, acyl, acyloxy, alkylthio, amino or aryl radicals, such as, for example, in chloromethyl, hydroxypropyl, methoxymethyl, propionyloxymethyl, methylthiomethyl, dimethylaminoethyl, benzyl or phenethyl groups.

The term “esterified carboxyl” means, for example, radicals such as alkyloxycarbonyl radicals, for example methoxycarbonyl, ethoxycarbonyl, propoxycarbonyl, butyl or tert-butyloxycarbonyl, cyclobutyloxycarbonyl, cyclopentyl-oxycarbonyl or cyclohexyloxycarbonyl.

Mention may also be made of radicals formed with readily cleavable ester residues, such as methoxymethyl or ethoxymethyl radicals; acyloxyalkyl radicals such as pivaloyloxymethyl, pivaloyloxyethyl, acetoxymethyl or acetoxyethyl; alkyloxycarbonyloxyalkyl radicals such as methoxycarbonyloxy methyl or ethyl radicals, and isopropyloxycarbonyloxy methyl or ethyl radicals.

A list of such ester radicals may be found, for example, in European patent EP 0 034 536.

The term “amidated carboxyl” means radicals of the type —CONR4R5 in which the radicals R4 and R5 have the meanings indicated above.

The term “alkylamino radical” NHalk means linear or branched methylamino, ethylamino, propylamino or butylamino radicals. Alkyl radicals containing not more than 4 carbon atoms are preferred, the alkyl radicals possibly being chosen from the alkyl radicals mentioned above.

The term “dialkylamino radical” N(alk)2 means radicals in which alk takes the values defined above: as previously, alkyl radicals containing not more than 4 carbon atoms, chosen from the list indicated above, are preferred. Examples that may be mentioned include dimethylamino, diethylamino and methylethylamino radicals.

The term “cyclic amine” denotes a 3- to 8-membered cycloalkyl radical in which a carbon atom is replaced with a nitrogen atom, the cycloalkyl radical having the meaning indicated above and also possibly containing one or more other heteroatoms chosen from O, S, SO2, N and NR3 with R3 as defined above: examples of such cyclic amines that may be mentioned include optionally substituted aziridyl, azetidinyl, pyrrolidinyl, piperidyl, morpholinyl, piperazinyl, indolinyl, pyrindolinyl and tetrahydroquinolyl radicals: mention is made more particularly of pyrrolidinyl, piperidyl and morpholinyl radicals.

The term “salified carboxyl” means the salts formed, for example, with one equivalent of sodium, potassium, lithium, calcium, magnesium or ammonium. Mention may also be made of the salts formed with organic bases such as methylamine, propylamine, trimethylamine, diethylamine and triethylamine. The sodium salt is preferred.

When the products of formula (I) comprise an amino radical that may be salified with an acid, it is clearly understood that these acid salts also form part of the invention. Mention may be made of the salts obtained, for example, with hydrochloric acid or methanesulfonic acid.

The addition salts with mineral or organic acids of the products of formula (I) may be, for example, the salts formed with hydrochloric acid, hydrobromic acid, hydriodic acid, nitric acid, sulfuric acid, phosphoric acid, propionic acid, acetic acid, trifluoroacetic acid, formic acid, benzoic acid, maleic acid, fumaric acid, succinic acid, tartaric acid, citric acid, oxalic acid, glyoxylic acid, aspartic acid, ascorbic acid, alkylmonosulfonic acids such as, for example, methane-sulfonic acid, ethanesulfonic acid or propanesulfonic acid, alkyldisulfonic acids such as, for example, methanedisulfonic acid or alpha,beta-ethanedisulfonic acid, arylmonosulfonic acids such as benzenesulfonic acid, and aryldisulfonic acids.

It may be recalled that stereoisomerism may be defined in its broad sense as the isomerism of compounds having the same structural formulae but whose various groups are arranged differently in space, especially such as in monosubstituted cyclohexanes whose substituent may be in an axial or equatorial position, and the various possible rotational conformations of ethane derivatives.

However, there is another type of stereoisomerism, due to the different spatial arrangements of fixed substituents, either on double bonds or on rings, which is often referred to as geometrical isomerism or cis-trans isomerism. The term “stereoisomer” is used in the present patent application in its broadest sense and thus relates to all the compounds indicated above.

A subject of the invention is especially the products of formula (I) as defined above, in which:

n represents the integer 0 or 2
R represents a pyridyl or pyrimidinyl radical substituted with
a radical NR1R2,
NR1R2 being such that R1 represents a hydrogen atom or an alkyl radical, and R2 is chosen from a hydrogen atom and alkyl radicals optionally substituted with a hydroxyl, aziridyl, azetidinyl, pyrrolidinyl, piperidyl, morpholinyl, or piperazinyl, which is itself optionally substituted on its second nitrogen atom with an alkyl radical; 3- to 6-membered cycloalkyl radicals; an optionally substituted phenyl radical; a pyrimidinyl radical; a pyridyl radical optionally substituted with a halogen atom; and the radical CO—R3 with R3 chosen from NR4R5 and optionally substituted alkoxy, piperidyl and phenyl radicals;
R4 and R5, which may be identical to or different from R1 and R2, are such that:
either one from among R4 and R5 represents a hydrogen atom or an alkyl radical, and the other from among R4 and R5 is chosen from a hydrogen atom and alkyl radicals optionally substituted with a hydroxyl, aziridyl, azetidinyl, pyrrolidinyl, piperidyl, morpholinyl, or piperazinyl, which is itself optionally substituted on its second nitrogen atom with an alkyl radical; 3- to 6-membered cycloalkyl radicals; an optionally substituted phenyl radical; a pyrimidinyl radical; a pyridyl radical optionally substituted with a halogen atom;
or R4 and R5 form, with the nitrogen atom to which they are attached, an aziridyl, azetidinyl, pyrrolidinyl, piperidyl, morpholinyl, or piperazinyl, which is itself optionally substituted on its second nitrogen atom with an alkyl radical, all the phenyl radicals being optionally substituted with one to three radicals, which may be identical or different, chosen from halogen atoms, alkyl radicals and radicals CO—NHAlk and CO—N(Alk)2;
the said products of formula (I) being in any possible racemic, enantiomeric or diastereoisomeric isomer form, and also the addition salts with mineral and organic acids or with mineral and organic bases of the said products of formula (I).

A subject of the invention is especially the products of formula (I) as defined above in which

n represents the integer 0 or 2
R represents a pyridyl or pyrimidinyl radical substituted with
a radical NR1R2,
NR1R2 being such that R1 represents a hydrogen atom or an alkyl radical containing one or two carbon atoms, and R2 is chosen from alkyl radicals containing 1 to 4 carbon atoms optionally substituted with a hydroxyl radical; an optionally substituted phenyl radical; a pyrimidinyl radical; a pyridyl radical optionally substituted with a halogen atom; and the radical CO—R3 with R3 chosen from piperidyl, optionally substituted phenyl, NH(alk) and N(alk)2; all the phenyl radicals being optionally substituted with one to three radicals, which may be identical or different, chosen from halogen atoms and alkyl radicals and radicals CO—NHAlk and CO—N(Alk)2;
the said products of formula (I) being in any possible racemic, enantiomeric or diastereoisomeric isomer form, and also the addition salts with mineral and organic acids or with mineral and organic bases of the said products of formula (I).

A subject of the invention is especially the products of formula (I) as defined above in which:

n represents the integer 0 or 2
R represents a pyridyl or pyrimidinyl radical substituted with
a radical NR1R2
in which R1 represents a hydrogen atom and R2 represents an isopropyl radical substituted with a hydroxyl radical; an optionally substituted phenyl radical; a pyrimidinyl radical; a pyridyl radical optionally substituted with a fluorine atom; or a radical CO—R3 with R3 chosen from piperidyl, optionally substituted phenyl, NHCH3 and N(CH3)2; all the phenyl radicals being optionally substituted with one to three radicals, which may be identical or different, chosen from chlorine and fluorine atoms, methyl radicals and radical CO—N(CH3)2; the said products of formula (I) being in any possible racemic, enantiomeric or diastereoisomeric isomer form, and also the addition salts with mineral and organic acids or with mineral and organic bases of the said products of formula (I).

A subject of the invention is especially the products of formula (I) as defined above in which n, Ra, Rb and R have the meanings given in any one of the preceding claims, in which the radicals NR1R2 or NR4R5 or alternatively NR1R2 and NR4R5 are chosen from the following radicals named ex 18 to ex 40:

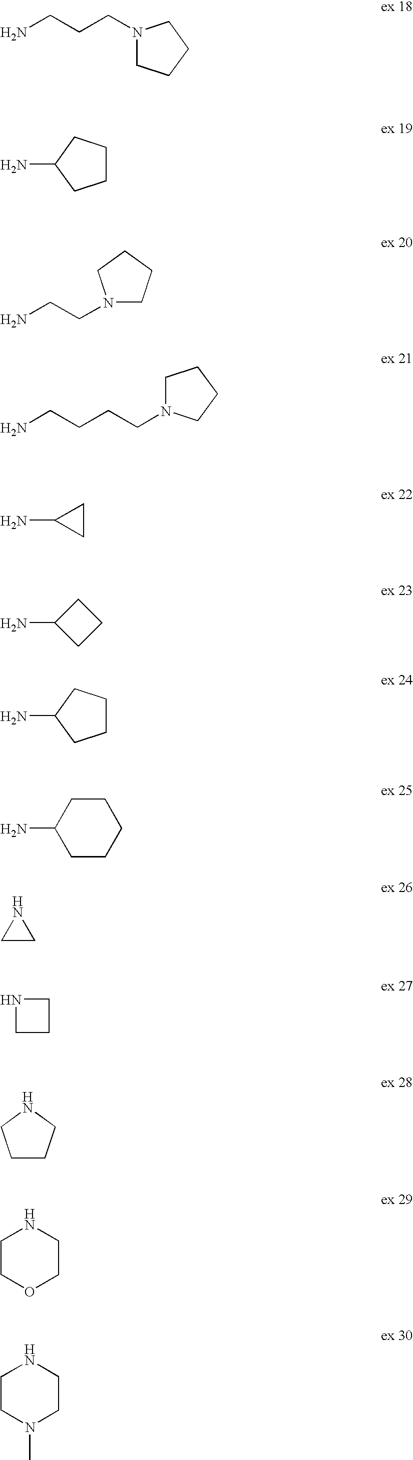

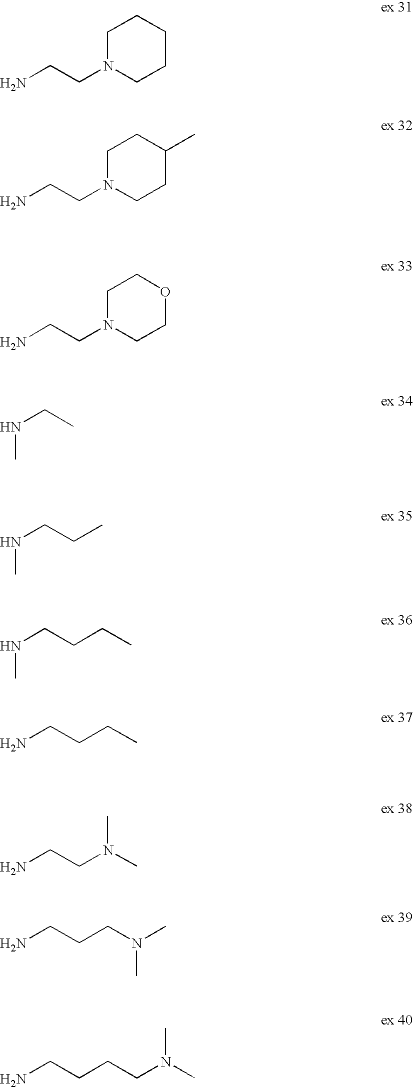

the said products of formula (I) being in any possible racemic, enantiomeric or diastereoisomeric isomer form, and also the addition salts with mineral and organic acids or with mineral and organic bases of the said products of formula (I).

A subject of the present invention is especially the products of formula (I) belonging to formula (I) as defined above in which the radical NR1R2 is chosen from the values ex 18 to ex 40:

A subject of the present invention is especially the products of formula (I) as defined above belonging to formula (Ia):

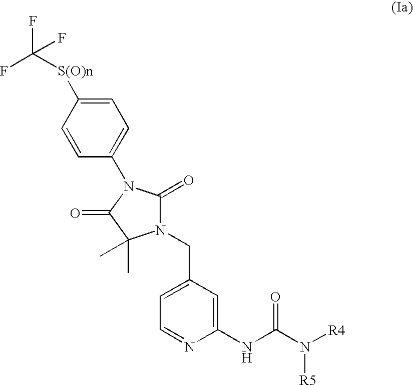

in which n and NR4R5 have the definitions given above and especially NR4R5 is chosen from the values ex 18 to ex 40 defined above,
the said products of formula (Ia) being in any possible racemic, enantiomeric or diastereoisomeric isomer form, and also the addition salts with mineral and organic acids or with mineral and organic bases of the said products of formula (Ia).

Among the preferred products of the invention, mention may be made more specifically of the products of formula (I) as defined above, whose names are as follows:
1-({2-[(2,5-dichlorophenyl)amino]pyridin-4-yl}methyl)-5,5-dimethyl-3-{4-[(trifluoromethyl)thio]phenyl}imidazolidine-2,4-dione
N-{4-[(5,5-dimethyl-2,4-dioxo-3-{4-[(trifluoromethyl)thio]-phenyl}imidazolidin-1-yl)methyl]pyridin-2-yl}piperidine-1-carboxamide
3,4-dichloro-N-{4-[(5,5-dimethyl-2,4-dioxo-3-{4-[(trifluoro-methyl)thio]phenyl}imidazolidin-1-yl)methyl]pyridin-2-yl}benzamide
1-{4-[(5,5-dimethyl-2,4-dioxo-3-{4-[(trifluoromethyl)-sulfonyl]phenyl}imidazolidin-1-yl)methyl]pyridin-2-yl}-3-methylurea
1-({2-[(2,5-difluorophenyl)amino]pyridin-4-yl}methyl)-5,5-dimethyl-3-{4-[(trifluoromethyl)thio]phenyl}imidazolidine-2,4-dione
3,5-dichloro-N-{4-[(5,5-dimethyl-2,4-dioxo-3-{4-[(trifluoro-methyl)thio]phenyl}imidazolidin-1-yl)methyl]pyridin-2-yl}benzamide
2-chloro-N-{4-[(5,5-dimethyl-2,4-dioxo-3-{4-[(trifluoro-methyl)thio]phenyl}imidazolidin-1-yl)methyl]pyridin-2-yl}-6-fluoro-3-methylbenzamide
3-({4-[(5,5-dimethyl-2,4-dioxo-3-{4-[(trifluoromethyl)thio]-phenyl}imidazolidin-1-yl)methyl]pyridin-2-yl}amino)-N,N-dimethylbenzamide
1-[(2-{[(1R)-2-hydroxy-1-methylethyl]amino}pyrimidin-4-yl)-methyl]-5,5-dimethyl-3-{4-[(trifluoromethyl)sulfonyl]phenyl}-imidazolidine-2,4-dione
3-{4-[(5,5-dimethyl-2,4-dioxo-3-{4-[(trifluoromethyl)thio]-phenyl}imidazolidin-1-yl)methyl]pyrimidin-2-yl}-1,1-dimethyl-urea
5,5-dimethyl-1-{[2-(pyridin-3-ylamino)pyrimidin-4-yl]methyl}-3-{4-[(trifluoromethyl)thio]phenyl}imidazolidine-2,4-dione
3-{4-[(5,5-dimethyl-2,4-dioxo-3-{4-[(trifluoromethyl)sulfonyl]phenyl}imidazolidin-1-yl)methyl]pyrimidin-2-yl}-1,1-dimethylurea
5,5-dimethyl-1-{[2-(pyrimidin-5-ylamino)pyridin-4-yl]methyl}-3-{4-[(trifluoromethyl)sulfonyl]phenyl}imidazolidine-2,4-dione
5,5-dimethyl-1-{[2-(pyrimidin-5-ylamino)pyridin-4-yl]methyl}-3-{4-[(trifluoromethyl)thio]phenyl}imidazolidine-2,4-dione
5,5-dimethyl-1-{[2-(pyrimidin-5-ylamino)pyrimidin-4-yl]-methyl}-3-{4-[(trifluoromethyl)thio]phenyl}imidazolidine-2,4-dione
5,5-dimethyl-1-{[2-(pyrimidin-5-ylamino)pyrimidin-4-yl]-methyl}-3-{4-[(trifluoromethyl)sulfonyl]phenyl}imidazolidine-2,4-dione
1-({2-[(5-fluoropyridin-3-yl)amino]pyridin-4-yl}methyl)-5,5-dimethyl-3-{4-[(trifluoromethyl)thio]phenyl}imidazolidine-2,4-dione
   the said products of formula (I) being in any possible racemic, enantiomeric or diastereoisomeric isomer form, and also the addition salts with mineral and organic acids or with mineral and organic bases of the said products of formula (I).

The products of formula (I) according to the present invention may be prepared according to the usual methods known to those skilled in the art.

The products of formula (I) according to the present invention may be prepared by application or adaptation of known methods and especially of the methods described in the literature, for instance those described by R. C. Larock in: Comprehensive Organic Transformations, VCH publishers, 1989.

The products according to the present invention may especially be prepared as indicated in General Scheme 1, consisting of General Scheme 1A and General Scheme 1B, General Scheme 2 and General Scheme 3 below.

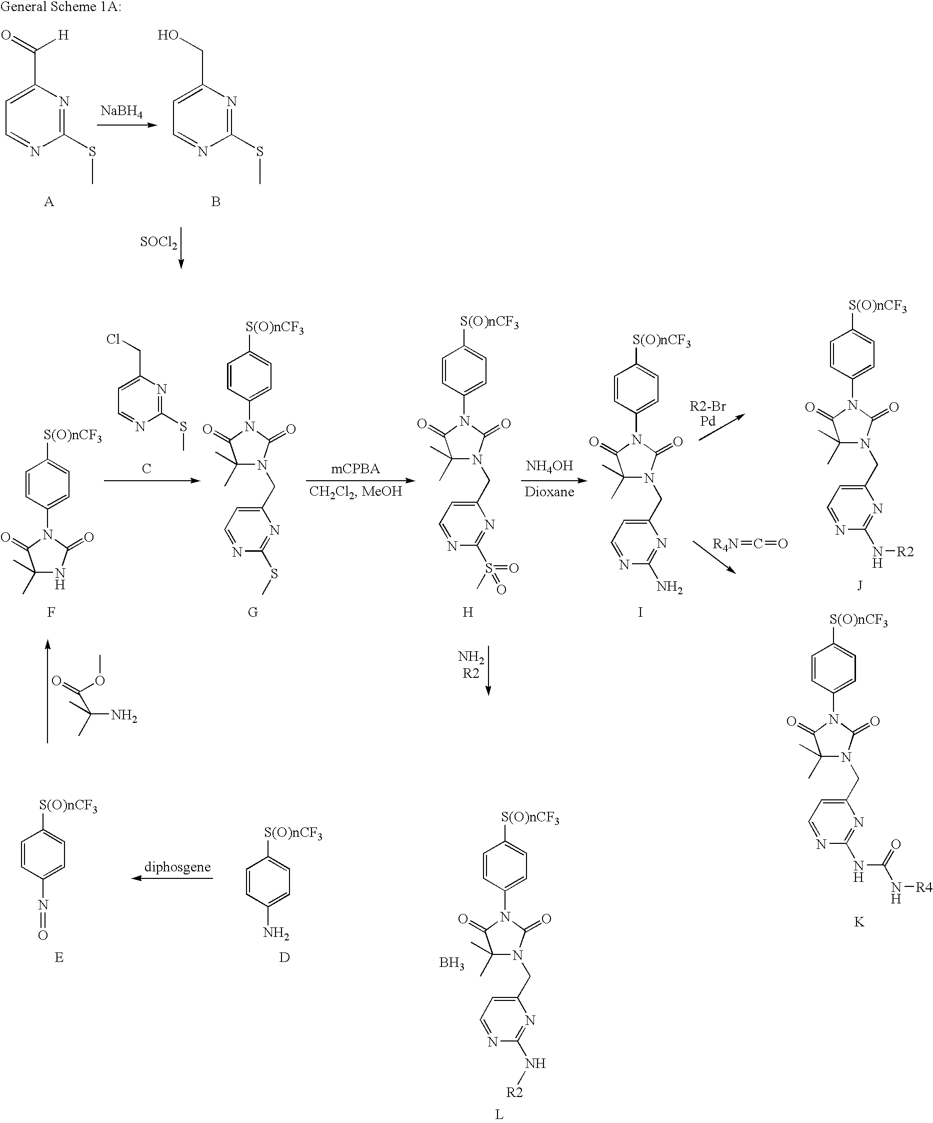

In General Scheme 1A:

The alcohol B may be obtained by treating the aldehyde A with a reducing agent such as sodium borohydride, in a solvent such as methanol at a temperature of between 0° C. and 60° C., for instance under the conditions described by Wang, E. et al. (Heterocycles 2002, 57(11), 2021-2033).

The chlorinated product C may be obtained from the alcohol B, for instance under the conditions described by Fucase K. et al. (Tetrahedron Lett., 1991, 32(32), 4019-4022) by treatment with thionyl chloride in the presence of DMF in a solvent such as dichloromethane at a temperature of between 0° C. and 20° C. The isocyanate E may be obtained from the anilines D by treatment with diphosgene in a solvent such as dioxane or toluene, for instance under the conditions described by Francis, J. E. et al. (J. Med. Chem. (1991), 34(1), 281-90). The hydantoin F may be obtained from the isocyanate E by reaction with methyl 2,2-dimethyl glycinate in a solvent such as toluene or N,N-dimethylformamide at a temperature of between 20° C. and the reflux temperature of the solvent, as described, for example, by Brana M. F. (J. Het. Chem. (2002), 39(2), 417-420.

The product G may be prepared by reacting the products F and C with sodium hydride in tetrahydrofuran or N,N-dimethylformamide at a temperature of between 0° C. and 60° C., as described by Johnson T. A. et al. (J. Am. Chem. Soc. (2002), 124, 11689-11698).

The product of general formula H may be prepared either by reacting G with meta-chloroperbenzoic acid in solvents such as a dichloromethane/methanol mixture (90:10; v/v) or 1,2-dichloroethane at temperatures of between 0° C. and 60° C. as described by Jeong, I. H. et al. (Bull. Korean Chem. Soc. (2002), 23 (12), 1823-1826).

Or by reaction of F with P (General Scheme 1B) in the presence of sodium hydride in tetrahydrofuran or N,N-dimethylformamide at a temperature of between 0° C. and 60° C. as described for the preparation of compound G.

The products of general formula I and L may be prepared by reacting H with ammonia dissolved in water and/or dioxane or with an amine (RNH2) dissolved in dioxane in a sealed microwave tube or by heating to temperatures of between 40° C. and 150° C., or as described by Font, D. et al. (Synthesis (2002), (13), 1833-1842).

The products of formula J may be prepared starting with I by reaction with an aryl or heteroaryl bromide (R2-Br) in the presence of a palladium-based catalyst such as palladium acetate and a ligand such as Xantphos (9,9-dimethyl-4,5-bis(diphenylphosphino)xanthene) in a solvent such as toluene, dioxane or tert-butanol, for instance under the conditions described Buchwald, S. L. et al. (J. Org. Chem. 2001, 66 (8), 2560-2565).

The products of general formula K may be obtained by reacting I with an isocyanate (R4-N═C═O) using the usual methods known to those skilled in the art.

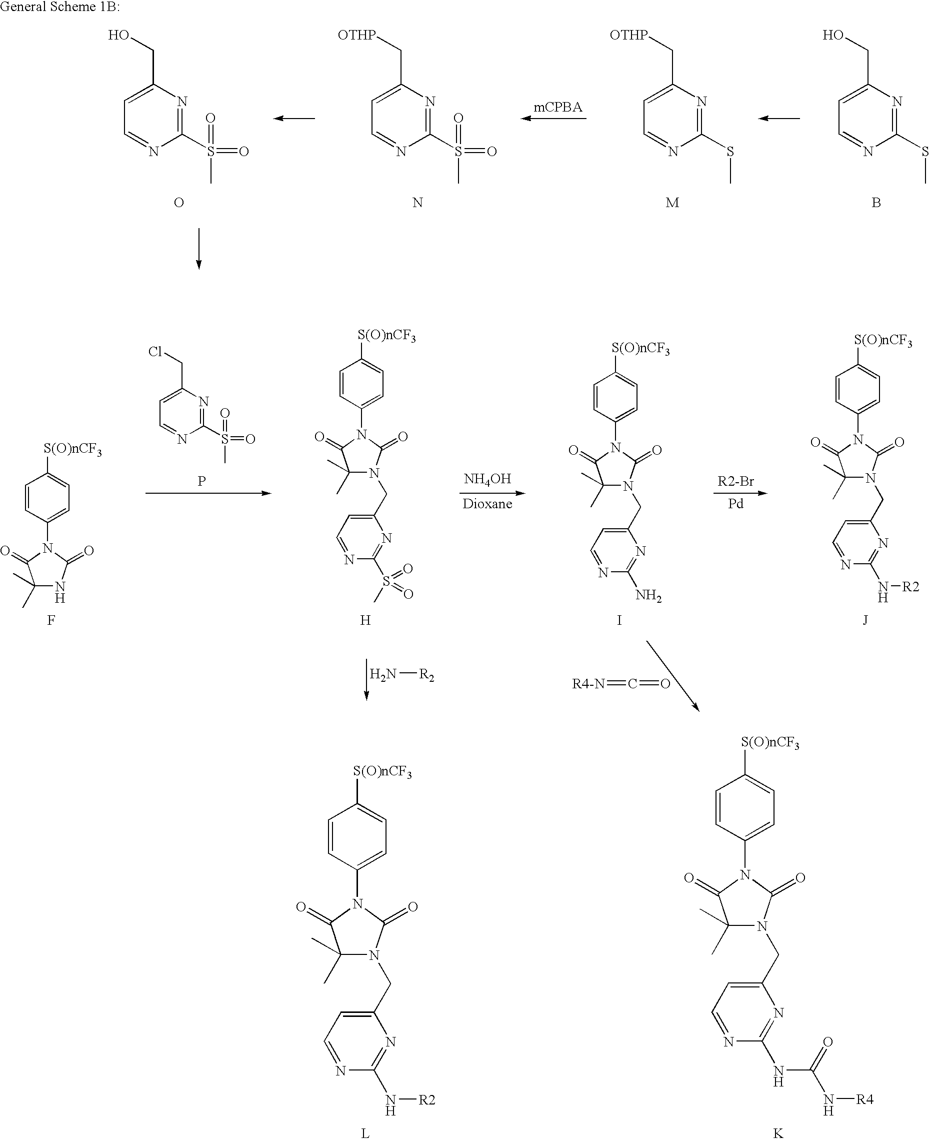

In General Scheme 1B:

The intermediates H for which n=2 can be prepared as described in General Scheme 1A, and the intermediates H for which n=0 can be prepared as described in General Scheme 1B. The product M can be obtained by treating the alcohol B with 3,4-dihydro-2H-pyran in dichloromethane in the presence of para-toluenesulfonic acid at a temperature of 20° C. as described by T. W. Greene et al. (Protective Groups in Organic Chemistry, John Wiley & Sons 1991, second edition).

The product N can be prepared by oxidizing sulphur, following the conditions described for the product H. The product 0 can be prepared by deprotecting the product N as described by T. W. Greene et al. (Protective Groups in Organic Chemistry, John Wiley & Sons 1991, second edition).

The product P can be prepared by halogenating the alcohol 0 as described in the preparation of the product C.

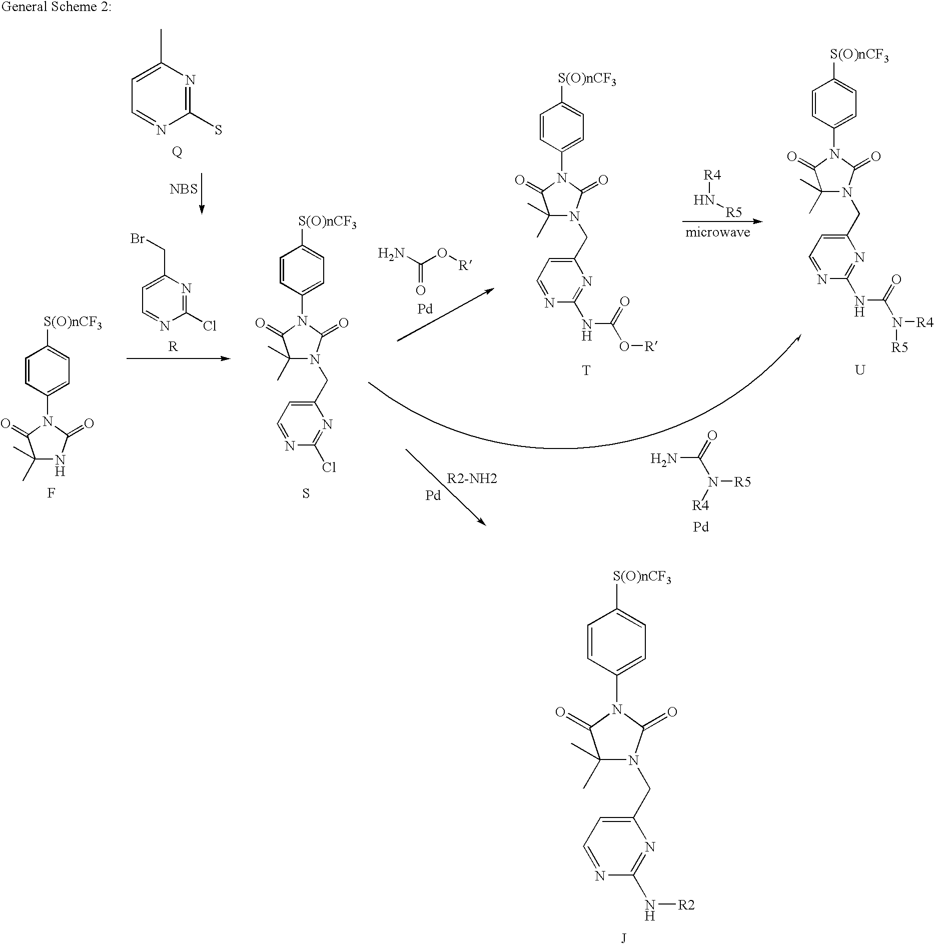

In General Scheme 2:

R′ represents alkyl or aryl as defined in R3 The product R may be prepared by bromination of product Q in the presence of N-bromosuccinimide in a solvent such as carbon tetrachloride as described by Brown, D. J. et al. (Aust. J. Chem. (1974), 2251).

Product S may be prepared from products R and F as described in the preparation of product G.

Product T may be prepared from S by reaction with a carbamate (NH2COOR′) in the presence of a palladium-based catalyst as described in the preparation of J.

Product U may be prepared either by reacting the carbamate T with an amine in a solvent such as N-methylpyrrolidinone or toluene at a temperature of between 50° C. and the reflux temperature of the solvent or by microwave, as described by Manov-Yuvenskii V. I et al. (Zh. Prikl. Khim. (1993), 66 (6), 1319-1327).

Or starting with S by reaction with a urea (NH2CONR4R5) in the presence of a palladium-based catalyst as described in the preparation of J.

Product J may be prepared from S by reaction with an amine (R2-NH2) in the presence of a palladium-based catalyst such as palladium acetate and a ligand such as Xantphos in a solvent such as toluene, dioxane or tert-butanol, for instance under the conditions described by Buchwald, S. L. et al. (J. Org. Chem. 2001, 66 (8), 2560-2565).

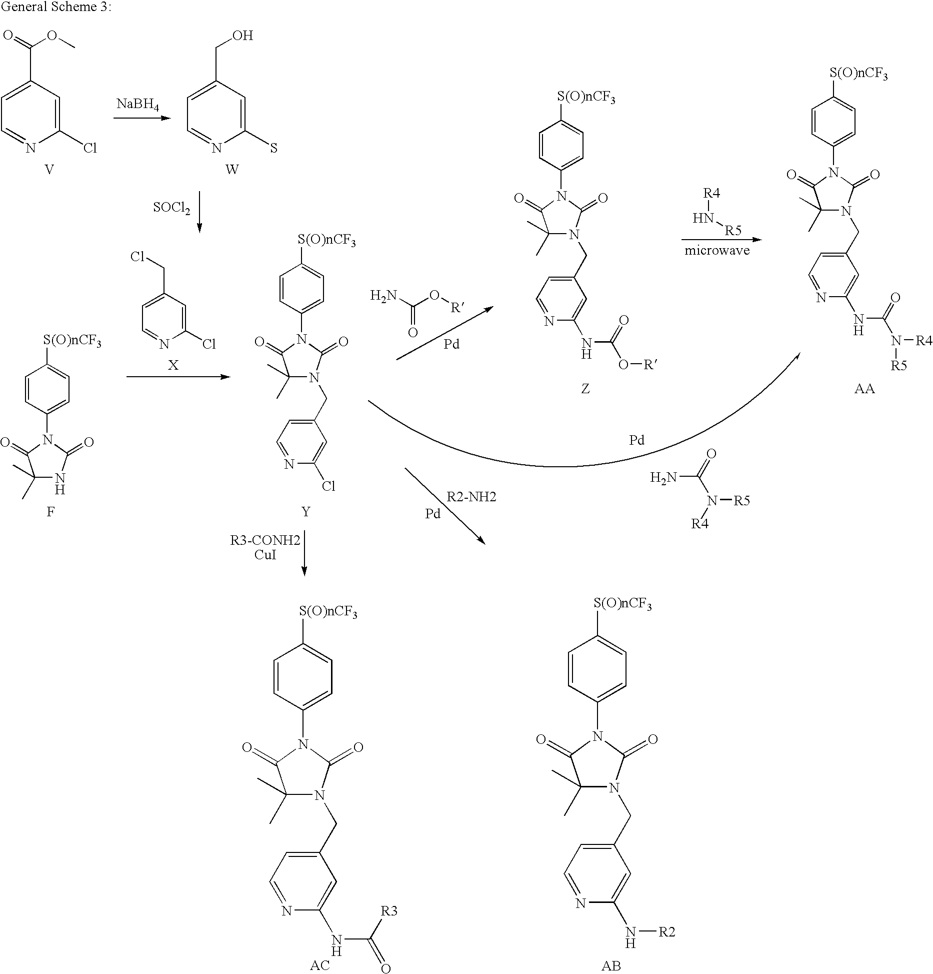

In the General Scheme 3:

R′ represents alkyl or aryl as defined in R3.

The alcohol W may be prepared by reduction of the ester V with a reducing agent such as sodium borohydride in a solvent such as ethanol at a temperature of between 20° C. and the reflux temperature of the solvent, as described by Zanka, A. et al. (Synlett (1999), (10), 1636-1638).

The product X is prepared by chlorination of the alcohol W as described in the preparation of C.

The product Y may be prepared from the products F and X using the conditions described for the preparation of G.

The product Z may be prepared from the product Y and the carbamate (NH2COOR′) using the conditions described for the preparation of J.

The product AA may be prepared either by reacting the product Z with an amine (NHR4R5) according to the conditions described for the product U,

or by reacting the product Y with a urea (NH2CONR4R5) according to the conditions described for the product J.

The product AB may be prepared from the product Y and the amine (NH2R2) according to the conditions described for the preparation of the product J.

The product AC may be prepared from the product Y and the amide (NH2COR3) in the presence of a copper catalyst, as described by Buchwald S. L. et al. (J. Am. Chem. Soc. (2001), 123, 7727-7729).

In such preparations of the products of formula (I) according to the present invention, the starting materials, the intermediates and the products of formula (I), which may be in protected form, may be subjected, if necessary or if desired, to one or more of the following transformations, in any order:

a) a reaction for esterification of an acid function,
b) a reaction for saponification of an ester function to an acid function,
c) a reaction for oxidation of an alkylthio group to the corresponding sulfoxide or sulfone group,
d) a reaction for conversion of a ketone function to an oxime function,
e) a reaction for reducing a free or esterified carboxyl function to an alcohol function,
f) a reaction for conversion of an alkoxy function to a hydroxyl function, or alternatively of a hydroxyl function to an alkoxy function,
g) a reaction for oxidation of an alcohol function to an aldehyde, acid or ketone function,
h) a reaction for conversion of a nitrile radical to a tetrazolyl,
i) a reaction for reduction of nitro compounds to amino compounds,
j) a reaction for removal of the protecting groups that may be borne by the protected reactive functions,
k) a reaction for salification with a mineral or organic acid or with a base to obtain the corresponding salt,
l) a reaction for resolution of the racemic forms to resolved products,
said products of formula (I) thus obtained being in any possible racemic, enantiomeric or diastereoisomeric isomer form.

It may be noted that such reactions for converting substituents into other substituents may also be performed on the starting materials, and also on the intermediates as defined above before continuing the synthesis according to the reactions indicated in the process described above.

In the reactions described below, it may be necessary to protect reactive functional groups, for instance hydroxyl, acyl, free carboxyl or amino and monoalkylamino radicals, imino, thio, etc., which may thus be protected with appropriate protecting groups.

Conventional protecting groups may be used in accordance with the usual standard practice, for instance those described, for example, by T. W. Greene and P. G. M. Wuts in “Protective Groups in Organic Chemistry”, John Wiley and Sons, 1991.

The following non-exhaustive list of examples of protection of reaction functions may be mentioned:

the hydroxyl groups may be protected, for example, with alkyl radicals such as tert-butyl, trimethylsilyl, tert-butyldimethylsilyl, methoxymethyl, tetrahydropyranyl, benzyl or acetyl,

the amino groups may be protected, for example, with acetyl, trityl, benzyl, tert-butoxycarbonyl, benzyloxycarbonyl, phthalimido radicals or other radicals known in peptide chemistry,

the acyl groups such as the formyl group may be protected, for example, in the form of cyclic or noncyclic ketals or thioketals such as dimethyl or diethylketal or ethylene dioxyketal, or diethylthioketal or ethylenedithioketal,

the acid functions of the products described above may be, if desired, amidated with a primary or secondary amine, for example in methylene chloride in the presence, for example, of 1-ethyl-3-(dimethylaminopropyl)carbodiimide hydrochloride at room temperature:

the acid functions may be protected, for example, in the form of esters formed with readily cleavable esters such as benzyl esters or tert-butyl esters, or esters known in peptide chemistry.

These reactions a) to k) indicated above may be performed, for example, as indicated below.

a) The products described above may, if desired, undergo, on the possible carboxyl functions, esterification reactions that may be performed according to the usual methods known to those skilled in the art.
b) The possible conversions of ester functions into an acid function of the products described above may be, if desired, performed under the usual conditions known to those skilled in the art, especially by acid or alkaline hydrolysis, for example with sodium hydroxide or potassium hydroxide in alcoholic medium such as, for example, in methanol, or alternatively with hydrochloric acid or sulfuric acid.
c) the possible alkylthio groups in the products described above, in which the alkyl radical is optionally substituted with one or more halogen atoms, especially fluorine, may, if desired, be converted into the corresponding sulfoxide or sulfone functions under the usual conditions known to those skilled in the art such as, for example, with peracids such as, for example, peracetic acid or meta-chloroperbenzoic acid, or alternatively with ozone, oxone or sodium periodate in a solvent such as, for example, methylene chloride or dioxane at room temperature.

The production of the sulfoxide function may be promoted with an equimolar mixture of the product containing an alkylthio group and the reagent such as, especially, a peracid.

The production of the sulfone function may be promoted with a mixture of the product containing an alkylthio group with an excess of the reagent such as, especially, a peracid.

d) The reaction for conversion of a ketone function into an oxime may be performed under the usual conditions known to those skilled in the art, such as, especially, a reaction in the presence of an optionally O-substituted hydroxylamine in an alcohol such as, for example, ethanol, at room temperature or with heating.
e) The possible free or esterified carboxyl functions of the products described above may be, if desired, reduced to an alcohol function by the methods known to those skilled in the art: the possible esterified carboxyl functions may be, if desired, reduced to an alcohol function by the methods known to those skilled in the art and especially with lithium aluminium hydride in a solvent such as, for example, tetrahydrofuran or dioxane or ethyl ether.

The possible free carboxyl functions of the products described above may be, if desired, reduced to an alcohol function especially with boron hydride.

f) The possible alkoxy functions such as, especially, methoxy, in the products described above, may be, if desired, converted into a hydroxyl function under the usual conditions known to those skilled in the art, for example with boron tribromide in a solvent such as, for example, methylene chloride, with pyridine hydrobromide or hydrochloride or with hydrobromic acid or hydrochloric acid in water or trifluoroacetic acid at reflux.
g) The possible alcohol functions of the products described above may be, if desired, converted into an aldehyde or acid function by oxidation under the usual conditions known to those skilled in the art, such as, for example, by the action of manganese oxide to obtain the aldehydes, or of Jones's reagent to access the acids.
h) The possible nitrile functions of the products described above may be, if desired, converted into tetrazolyl under the usual conditions known to those skilled in the art, such as, for example, by cycloaddition of a metal azide such as, for example, sodium azide or a trialkyltin azide on the nitrile function, as indicated in the method described in the article referenced as follows:

### J. Organometallic Chemistry., 33, 337 (1971) KOZIMA S. et al.

It may be noted that the reaction for conversion of a carbamate into urea and especially of a sulfonylcarbamate into sulfonylurea may be performed, for example, at the reflux point of a solvent such as, for example, toluene, in the presence of the appropriate amine.

It is understood that the reactions described above may be performed as indicated or alternatively, where appropriate, according to other common methods known to those skilled in the art.

i) The removal of protecting groups such as, for example, those indicated above may be performed under the usual conditions known to those skilled in the art, especially via an acid hydrolysis performed with an acid such as hydrochloric acid, benzenesulfonic acid or para-toluenesulfonic acid, formic acid or trifluoroacetic acid, or alternatively via a catalytic hydrogenation.

The phthalimido group may be removed with hydrazine.

A list of various protecting groups that may be used will be found, for example, in patent BF 2 499 995.

j) The products described above may, if desired, be subjected to salification reactions, for example with a mineral or organic acid or with a mineral or organic base according to the usual methods known to those skilled in the art.
k) The possible optically active forms of the products described above may be prepared by resolving the racemic mixtures according to the usual methods known to those skilled in the art.

The possible reactive functions that are optionally protected are especially the hydroxyl or amino functions. Usual protecting groups are used to protect these functions. Examples that may be mentioned include the following protecting groups for the amino radical: tert-butyl, tert-amyl, trichloroacetyl, chloroacetyl, benzhydryl, trityl, formyl, benzyloxycarbonyl.

Protecting groups for the hydroxyl radical that may be mentioned include radicals such as formyl, chloroacetyl, tetrahydropyranyl, trimethylsilyl and tert-butyldimethylsilyl.

It is clearly understood that the above list is not limiting and that other protecting groups, which are known, for example, in peptide chemistry, may be used. A list of such protecting groups is found, for example, in French patent BF 2 499 995, the content of which is incorporated herein by reference.

The possible reactions for removal of the protecting groups are performed as indicated in said patent BF 2 499 995. The preferred method of removal is acid hydrolysis with acids chosen from hydrochloric acid, benzenesulfonic acid or para-toluenesulfonic acid, formic acid or trifluoroacetic acid. Hydrochloric acid is preferred.

The possible reaction for hydrolysis of the >C═NH group to a ketone group is also preferably performed using an acid such as aqueous hydrochloric acid, for example at reflux.

An example of removal of the tert-butyldimethylsilyl group using hydrochloric acid is given below in the examples.

The possible esterification of a free OH radical is performed under standard conditions. An acid or a functional derivative, for example an anhydride such as acetic anhydride in the presence of a base such as pyridine may be used, for example.

The possible esterification or salification of a COOH group is performed under the standard conditions known to those skilled in the art.

The possible amidation of a COOH radical is performed under standard conditions. A primary or secondary amine may be used on a functional derivative of the acid, for example a symmetrical or mixed anhydride.

The starting materials used for the preparation of the products of formula (I) according to the present invention may be known and commercially available or may be prepared according to methods known to those skilled in the art.

The products that are the subject of the present invention have advantageous pharmacological properties: it has been found that they especially have inhibitory properties on protein kinases.

Among these protein kinases, mention may be made especially of IGF1R.

Tests given in the experimental section below illustrate the inhibitory activity of products of the present invention with respect to such protein kinases.

These properties thus make the products of general formula (I) of the present invention usable as medicaments for treating malignant tumours.

The products of formula (I) may also be used in the veterinary field.

A subject of the invention is thus the use, as medicaments, of the pharmaceutically acceptable products of general formula (I).

A subject of the invention is particularly the use, as medicaments, of the products whose names are as follows:
1-({2-[(2,5-dichlorophenyl)amino]pyridin-4-yl}methyl)-5,5-dimethyl-3-{4-[(trifluoromethyl)thio]phenyl}imidazolidine-2,4-dione
N-{4-[(5,5-dimethyl-2,4-dioxo-3-{4-[(trifluoromethyl)thio]-phenyl}imidazolidin-1-yl)methyl]pyridin-2-yl}piperidine-1-carboxamide
3,4-dichloro-N-{4-[(5,5-dimethyl-2,4-dioxo-3-{4-[(trifluoro-methyl)thio]phenyl}imidazolidin-1-yl)methyl]pyridin-2-yl}benzamide
1-{4-[(5,5-dimethyl-2,4-dioxo-3-{4-[(trifluoromethyl)-sulfonyl]phenyl}imidazolidin-1-yl)methyl]pyridin-2-yl}-3-methylurea
1-({2-[(2,5-difluorophenyl)amino]pyridin-4-yl}methyl)-5,5-dimethyl-3-{4-[(trifluoromethyl)thio]phenyl}imidazolidine-2,4-dione
3,5-dichloro-N-{4-[(5,5-dimethyl-2,4-dioxo-3-{4-[(trifluoro-methyl)thio]phenyl}imidazolidin-1-yl)methyl]pyridin-2-yl}benzamide
2-chloro-N-{4-[(5,5-dimethyl-2,4-dioxo-3-{4-[(trifluoro-methyl)thio]phenyl}imidazolidin-1-yl)methyl]pyridin-2-yl}-6-fluoro-3-methylbenzamide
3-({4-[(5,5-dimethyl-2,4-dioxo-3-{4-[(trifluoromethyl)thio]-phenyl}imidazolidin-1-yl)methyl]pyridin-2-yl}amino)-N,N-dimethylbenzamide
1-[(2-{[(1R)-2-hydroxy-1-methylethyl]amino}pyrimidin-4-yl)-methyl]-5,5-dimethyl-3-{4-[(trifluoromethyl)sulfonyl]phenyl}-imidazolidine-2,4-dione
3-{4-[(5,5-dimethyl-2,4-dioxo-3-{4-[(trifluoromethyl)thio]-phenyl}imidazolidin-1-yl)methyl]pyrimidin-2-yl}-1,1-dimethyl-urea
5,5-dimethyl-1-{[2-(pyridin-3-ylamino)pyrimidin-4-yl]methyl}-3-{4-[(trifluoromethyl)thio]phenyl}imidazolidine-2,4-dione
3-{4-[(5,5-dimethyl-2,4-dioxo-3-{4-[(trifluoromethyl)-sulfonyl]phenyl}imidazolidin-1-yl)methyl]pyrimidin-2-yl}-1,1-dimethylurea
5,5-dimethyl-1-{[2-(pyrimidin-5-ylamino)pyridin-4-yl]methyl}-3-{4-[(trifluoromethyl)sulfonyl]phenyl}imidazolidine-2,4-dione
5,5-dimethyl-1-{[2-(pyrimidin-5-ylamino)pyridin-4-yl]methyl}-3-{4-[(trifluoromethyl)thio]phenyl}imidazolidine-2,4-dione
5,5-dimethyl-1-{[2-(pyrimidin-5-ylamino)pyrimidin-4-yl]-methyl}-3-{4-[(trifluoromethyl)thio]phenyl}imidazolidine-2,4-dione
5,5-dimethyl-1-{[2-(pyrimidin-5-ylamino)pyrimidin-4-yl]-methyl}-3-{4-[(trifluoromethyl)sulfonyl]phenyl}imidazolidine-2,4-dione
1-({2-[(5-fluoropyridin-3-yl)amino]pyridin-4-yl}methyl)-5,5-dimethyl-3-{4-[(trifluoromethyl)thio]phenyl}imidazolidine-2,4-dione
   the said products of formula (I) being in any possible racemic, enantiomeric or diastereoisomeric isomer form, and also the pharmaceutically acceptable addition salts with mineral and organic acids or with mineral and organic bases of the said products of formula (I).

The products may be administered parenterally, orally, perlingually, rectally or topically.

A subject of the invention is also pharmaceutical compositions, characterized in that they contain as active principle at least one of the medicaments of general formula (I).

These compositions may be in the form of injectable solutions or suspensions, tablets, coated tablets, capsules, syrups, suppositories, creams, ointments and lotions. These pharmaceutical forms are prepared according to the usual methods. The active principle may be incorporated into excipients usually used in these compositions, such as aqueous or nonaqueous vehicles, talc, gum arabic, lactose, starch, magnesium stearate, cocoa butter, fatty substances of animal or plant origin, paraffin derivatives, glycols, various wetting, dispersing or emulsifying agents, and preserving agents.

The usual dose, which varies according to the individual treated and the complaint under consideration, may be, for example, from 10 mg to 500 mg per day orally in man.

The present invention thus relates to the use of products of formula (I) as defined above or of pharmaceutically acceptable salts of said products of formula (I) for the preparation of medicaments for inhibiting the activity of protein kinases and especially of a protein kinase.

The present invention thus relates to the use of products of formula (I) as defined above or of pharmaceutically acceptable salts of said products of formula (I) in which the protein kinase is a protein tyrosine kinase.

The present invention thus relates to the use of products of formula (I) as defined above or of pharmaceutically acceptable salts of said products of formula (I) in which the protein kinase is chosen from the following group: EGFR, Fak, FLK-1, FGFR1, FGFR2, FGFR3, FGFR4, FGFR5, flt-1, IGF-1R, KDR, PDGFR, tie2, VEGFR, AKT, Raf.

The present invention thus relates particularly to the use of products of formula (I) as defined above or of pharmaceutically acceptable salts of said products of formula (I) in which the protein kinase is IGF1R.

The present invention also relates to the use of products of formula (I) as defined above or of pharmaceutically acceptable salts of said products of formula (I) in which the protein kinase is in a cell culture, and also to this use in a mammal.

The present invention thus relates to the use of products of formula (I) as defined above or of pharmaceutically acceptable salts of said products of formula (I) for the preparation of a medicament for preventing or treating a disease characterized by deregulation of the activity of a protein kinase and especially such a disease in a mammal.

The present invention relates to the use of products of formula (I) as defined above or of pharmaceutically acceptable salts of said products of formula (I) for the preparation of a medicament for preventing or treating a disease belonging to the following group: disorders of blood vessel proliferation, fibrotic disorders, disorders of mesangial cell proliferation, metabolic disorders, allergies, asthma, thrombosis, diseases of the nervous system, retinopathy, psoriasis, rheumatoid arthritis, diabetes, muscle degeneration, oncology diseases and cancer.

The present invention thus relates to the use of products of formula (I) as defined above or of pharmaceutically acceptable salts of said products of formula (I) for the preparation of a medicament for treating oncology diseases.

The present invention relates particularly to the use of products of formula (I) as defined above or of pharmaceutically acceptable salts of said products of formula (I) for the preparation of a medicament for treating cancers.

Among these cancers, the present invention is most particularly of interest in the treatment of solid tumours and the treatment of cancers that are resistant to cytotoxic agents.

Among these cancers, the present invention relates most particularly to the treatment of breast cancer, stomach cancer, cancer of the colon, lung cancer, cancer of the ovaries, cancer of the uterus, brain cancer, cancer of the kidney, cancer of the larynx, cancer of the lymphatic system, cancer of the thyroid, cancer of the urogenital tract, cancer of the tract including the seminal vesicle and prostate, bone cancer, cancer of the pancreas and melanomas.

The present invention is even more particularly of interest in treating breast cancer, cancer of the colon and lung cancer.

The present invention also relates to the use of products of formula (I) as defined above or of pharmaceutically acceptable salts of said products of formula (I) for the preparation of a medicament for cancer chemotherapy.

As medicaments according to the present invention for cancer chemotherapy, the products of formula (I) according to the present invention may be used alone or in combination with chemotherapy or radiotherapy or alternatively in combination with other therapeutic agents.

The present invention thus relates especially to the pharmaceutical compositions as defined above, also containing active principles of other chemotherapy medicaments for combating cancer.

Such therapeutic agents may be commonly used antitumour agents.

As examples of known inhibitors of protein kinases, mention may be made especially of butyrolactone, flavopiridol, 2-(2-hydroxyethylamino)-6-benzylamino-9-methylpurine, olomucine, Glivec and Iressa.

The products of formula (I) according to the present invention may thus also be advantageously used in combination with antiproliferative agents: as examples of such antiproliferative agents, but without, however, being limited to this list, mention may be made of aromatase inhibitors, antioestrogens, the topoisomerase I inhibitors, the topoisomerase II inhibitors, microtubule-active agents, alkylating agents, histone deacetylase inhibitors, farnesyl transferase inhibitors, COX-2 inhibitors, MMP inhibitors, mTOR inhibitors, antineoplastic antimetabolites, platinum compounds, compounds that reduce the activity of protein kinases and also anti-angiogenic compounds, gonadorelin agonists, antiandrogens, bengamides, biphosphonates and trastuzumab.

Examples that may thus be mentioned include anti-microtubule agents, for instance taxoids, vinca alkaloids, alkylating agents such as cyclophosphamide, DNA-intercalating agents, for instance cis-platinum, agents that are interactive on topoisomerase, for instance camptothecin and derivatives, anthracyclines, for instance adriamycin, antimetabolites, for instance 5-fluorouracil and derivatives, and the like.

The present invention thus relates to products of formula (I) as protein kinase inhibitors, said products of formula (I) being in any possible racemic, enantiomeric or diastereoisomeric isomer form, and also the addition salts with pharmaceutically acceptable mineral and organic acids or with pharmaceutically acceptable mineral and organic bases of said products of formula (I), and also the prodrugs thereof.

The present invention relates particularly to products of formula (I) as defined above, as IGF1R inhibitors.

The present invention relates more particularly to the products of formula (I) as defined above as IGF1R inhibitors.

The 1H NMR spectra are recorded on Brüker spectrometers at 400 MHz (AVANCE DRX-400) or at 300 MHz (BRUKER AVANCE DPX-300). The chemical shifts are given in ppm (δ in ppm)—in the solvent dimethyl sulfoxide-d6 (DMSO-d6) reference to 2.50 ppm at a temperature of 303K.

The mass spectra were acquired either by electrospray (ES) on a Q-Tof-2 (Micromass), ZQ (Micromass) or Quattro Premier (Micromass) machine, or by electron impact (EI); 70 eV; Micromass GCT of Premier machine, or by chemical ionization (CI); reactor and gas: ammonia; Micromass GCT of machine.

The LCMS is performed on a Hypersil Gold C18 column 3×50 mm in diameter; particles: 3 μm initial conditions:

Solvent A: water containing 0.05% TFA 95%
Solvent B: acetonitrile containing 0.05% TFA 5%
Flow rate 0.9 mL; pressure at t₀: 145b; volume injected: 5 μl
GRADIENT over 7 minutes

| Time | % A | % B |
|---|---|---|
| 0 | 95 | 5 |
| 5 | 5 | 95 |
| 5.5 | 5 | 95 |
| 6.5 | 95 | 5 |
| 7 | 95 | 5 |

DAD UV detector: 200<λ<400 nm, the mass is measured by electrospray (ES+) on a Q-Tof-2 machine (Micromass).

The examples whose preparation follows illustrate the present invention without, however, limiting it.

### EXAMPLE 1

### 1-({2-[(2,5-dichlorophenyl)amino]pyridin-4-yl}-methyl)-5,5-dimethyl-3-{4-[(trifluoromethyl)thio]phenyl}imidazolidine-2,4-dione

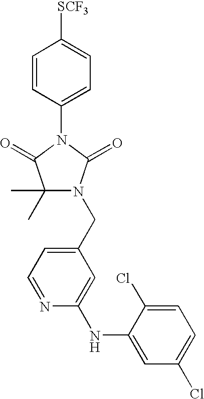

### Stage e)

### 1-({2-[(2,5-dichlorophenyl)amino]pyridin-4-yl}-methyl)-5,5-dimethyl-3-{4-[(trifluoromethyl)thio]phenyl}imidazolidine-2,4-dione

To a solution of 0.8 g of 1-[(2-chloropyridin-4-yl)methyl]-5,5-dimethyl-3-{4-[(trifluoromethyl)thio]phenyl}imidazolidine-2,4-dione obtained in stage d) below, 80 cm³ of toluene and 0.45 g of 2,5-dichloroaniline, under an inert atmosphere of argon at a temperature in the region of 20° C., are added 0.17 g of palladium acetate, 0.48 g of 9,9-dimethyl-4,5-bis(diphenyl-phosphino)xanthene and 2.4 g of caesium carbonate. The reaction medium is refluxed for 18 hours. After cooling, the reaction medium is concentrated under reduced pressure. The residue obtained is purified by flash chromatography (SiO2, dichloro-methane as eluent). The fractions containing the product are concentrated under reduced pressure. 0.46 g of 1-({2-[(2,5-dichlorophenyl)amino]pyridin-4-yl}methyl)-5,5-dimethyl-3-{4-[(trifluoromethyl)thio]phenyl}imidazolidine-2,4-dione is thus obtained, the characteristics of which are as follows:

1H NMR spectrum at 400 MHz: 1.45 (s, 6H); 4.58 (s, 2H); 6.92 (broad d, J=5.5 Hz, 1H); 7.03 (dd, J=2.5 and 8.5 Hz, 1H); 7.13 (broad s, 1H); 7.47 (d, J=8.5 Hz, 1H); 7.69 (d, J=9.0 Hz, 2H); 7.88 (d, J=9.0 Hz, 2H); 8.15 (d, J=5.5 Hz, 1H); 8.36 (d, J=2.5 Hz, 1H); 8.46 (s, 1H)

Mass Spectrum (ES): m/z=555 [M+H]⁺ base peak

### Stage d)

### 1-[(2-chloropyridin-4-yl)methyl]-5,5-dimethyl-3-{4-[(trifluoromethyl)thio]phenyl}imidazolidine-2,4-dione

To a solution of 5 g of 5,5-dimethyl-3-{4-[(trifluoromethyl)-thio]phenyl}imidazolidine-2,4-dione obtained in stage c) below in 220 mL of anhydrous THF, under an inert atmosphere of argon at a temperature in the region of 20° C., is added 0.9 g of sodium hydride, stirring is continued at this temperature for 30 minutes, and a solution of 3 g of 2-chloro-4-(chloro-methyl)pyridine obtained in stage b) below in 10 mL of anhydrous THF is added. The reaction medium is heated at 60° C. for 48 hours. The reaction medium is poured onto ice and then extracted with ethyl acetate. The organic phase is dried over magnesium sulfate, filtered, concentrated under vacuum and then purified by chromatography on 40-60 μm silica (eluents: dichloromethane/ethyl acetate 97/03 by volume). The fractions containing the product are concentrated under reduced pressure. 1.17 g of 1-[(2-chloropyridin-4-yl)methyl]-5,5-dimethyl-3-{4-[(trifluoromethyl)thio]phenyl}imidazolidine-2,4-dione are thus obtained in the form of a white powder, the characteristics of which are as follows:

m.p. 111° C.

Mass Spectrum (IC): m/z=447 MNH₄⁺, m/z=430 [M+H]⁺ base peak

### Stage c)

### 5,5-dimethyl-3-{4-[(trifluoromethyl)thio]phenyl}imidazolidine-2,4-dione

To a solution of 4 g of 4-(trifluoromethyl)thiophenyl isocyanate in 40 mL of toluene, under an inert atmosphere of argon at a temperature in the region of 20° C., are added 5.12 mL of triethylamine and 2.8 g of methyl α-aminoisobutyrate hydrochloride. The mixture thus obtained is refluxed for 24 hours and then cooled to room temperature. The reaction mixture is concentrated to dryness under reduced pressure, and the residue obtained is taken up in ethyl ether and filtered. The solid thus obtained is taken up in dichloromethane and then washed with water to give 2.76 g of 5,5-dimethyl-3-{4-[(tri-fluoromethyl)thio]phenyl}imidazolidine-2,4-dione, the characteristics of which are as follows:

1H NMR spectrum at 300 MHz: 1.44 (s: 6H); 7.62 (broad d, J=8.5 Hz: 2H); 7.85 (broad d, J=8.5 Hz: 2H); 8.72 (complex: 1H).

Mass Spectrum (IC): m/z=322 MNH₄⁺

### Stage b)

### 2-chloro-4-(chloromethyl)pyridine

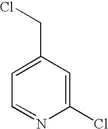

To a solution of 11.3 g of (2-chloropyridin-4-yl)methanol obtained in stage a) below in 200 mL of dichloromethane are added 6.896 mL of thionyl chloride and then 2.1 mL of dimethylformamide, the reaction mixture is stirred for 3 hours and 50 mL of water are then added dropwise. The solution is dried over magnesium sulfate, filtered and concentrated under vacuum to give 12.8 g (100%) of product in the form of an amber-coloured liquid, which is used without further purification.

R_{f}TLC silica=0.44 (eluent: dichloromethane).

### Stage a)

### (2-Chloropyridin-4-yl)methanol

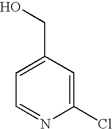

To a solution of 14.85 g of ethyl 2-chloroisonicotinate in 300 mL of ethanol are added, under argon, 9.08 g of sodium borohydride portionwise at 40° C. for 45 minutes. After addition, the reaction mixture is stirred for 15 minutes and the temperature is then gradually raised to reflux, which is maintained for 4 hours. After cooling to room temperature, 50 mL of saturated ammonium chloride solution are added and the solvents are evaporated off under reduced pressure. The residue is taken up in 200 mL of water and extracted with 3×100 mL of ethyl acetate, and the organic phase is washed with 2×100 mL of saturated sodium chloride solution, dried over sodium sulfate and filtered. After evaporation under reduced pressure, the product is obtained in the form of a white solid: 11.4 g.

R_{f} TLC silica=0.38 (eluent: dichloromethane/methanol 90/10).

### EXAMPLE 2

### N-{4-[(5,5-dimethyl-2,4-dioxo-3-{4-[(trifluoro-methyl)thio]phenyl}imidazolidin-1-yl)methyl]pyridin-2-yl}piperidine-1-carboxamide

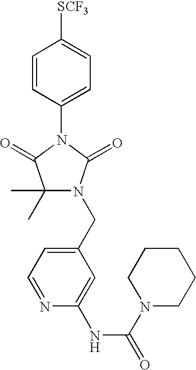

The product is prepared according to the procedure described in Example 1, starting with 0.4 g of 1-[(2-chloropyridin-4-yl)-methyl]-5,5-dimethyl-3-{4-[(trifluoromethyl)thio]phenyl}imidazolidine-2,4-dione obtained in stage d) of Example 1 and 0.18 g of 1-piperidinecarboxamide instead of the 2,5-dichloroaniline used in Example 1. After purification by flash-pack chromatography (SiO2, dichloromethane/methanol 98/02 by volume as eluents), 0.21 g of N-{4-[(5,5-dimethyl-2,4-dioxo-3-{4-[(trifluoromethyl)thio]phenyl}imidazolidin-1-yl)methyl]pyridin-2-yl}piperidine-1-carboxamide is obtained, the characteristics of which are as follows:

1H NMR spectrum at 400 MHz: from 1.39 to 1.60 (m, 6H); 1.41 (s, 6H); 3.42 (m, 4H); 4.59 (s, 2H); 7.01 (dd, J=1.0 and 5.5 Hz, 1H); 7.67 (d, J=8.5 Hz, 2H); 7.82 (broad s, 1H); 7.86 (d, J=8.5 Hz, 2H); 8.16 (d, J=5.5 Hz, 1H); 9.05 (s, 1H).

Mass Spectrum (ES): m/z=522 [M+H]⁺ base peak

### EXAMPLE 3

### 3,4-dichloro-N-{4-[(5,5-dimethyl-2,4-dioxo-3-{4-[(trifluoromethyl)thio]phenyl}imidazolidin-1-yl)methyl]pyridin-2-yl}benzamide

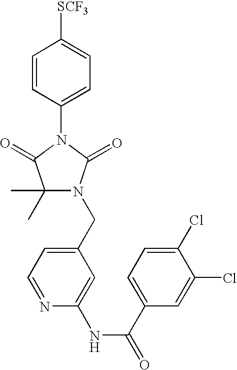

To a solution of 0.7 g of 1-[(2-chloropyridin-4-yl)methyl]-5,5-dimethyl-3-{4-[(trifluoromethyl)thio]phenyl}imidazolidine-2,4-dione obtained in stage d) of Example 1, 70 cm³ of dioxane and 0.63 g of 3,4-dichlorobenzamide, under an inert atmosphere of argon at a temperature in the region of 20° C., are added 0.16 g of copper iodide, 0.11 g of bis-methylcyclohexanediamine and 0.665 g of potassium carbonate. The reaction medium is refluxed for 18 hours. After cooling, the reaction medium is concentrated under reduced pressure. The residue obtained is purified by flash chromatography (SiO2, dichloromethane/ethyl acetate 95/05 by volume as eluents). The fractions containing the product are concentrated under reduced pressure. 0.49 g of 3,4-dichloro-N-{4-[(5,5-dimethyl-2,4-dioxo-3-{4-[(trifluoro-methyl)thio]phenyl}imidazolidin-1-yl)methyl]pyridin-2-yl}-benzamide is thus obtained, the characteristics of which are as follows:

1H NMR spectrum at 400 MHz: 1.45 (s, 6H); 4.69 (s, 2H); 7.25 (dd, J=1.5 and 5.5 Hz, 1H); 7.67 (d, J=8.5 Hz, 2H); 7.79 (d, J=8.5 Hz, 1H); 7.87 (d, J=8.5 Hz, 2H); 7.98 (dd, J=2.0 and 8.5 Hz, 1H); 8.21 (broad s, 1H); 8.28 (d, J=2.0 Hz, 1H); 8.36 (d, J=5.5 Hz, 1H); 11.05 (s, 1H).

Mass Spectrum (ES): m/z=583 [M+H]⁺ base peak

### EXAMPLE 4

### 1-{4-[(5,5-dimethyl-2,4-dioxo-3-{4-[(trifluoro-methyl)sulfonyl]phenyl}imidazolidin-1-yl)methyl]pyridin-2-yl}-3-methylurea

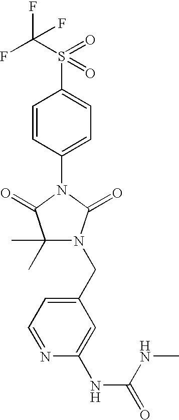

### Stage c)

### 1-{4-[(5,5-dimethyl-2,4-dioxo-3-{4-[(trifluoro-methyl)sulfonyl]phenyl}imidazolidin-1-yl)methyl]pyridin-2-yl}-3-methylurea

To a solution of 0.69 g of 1-[(2-chloropyridin-4-yl)methyl]-5,5-dimethyl-3-{4-[(trifluoromethyl)sulfonyl]phenyl}imidazolidine-2,4-dione obtained in stage b) above in 20 mL of dioxane are successively added, under argon, 0.166 g of methylurea, 1.85 g of caesium carbonate, 0.104 g of 9,9-dimethyl-4,5-bis(diphenylphosphino)xanthene and 0.33 g of palladium acetate. The reaction mixture is refluxed for 2.5 hours and then concentrated under reduced pressure and the residue is purified by chromatography on a column of silica, eluting with a mixture of cyclohexane and ethyl acetate (20/80 by volume) to give 0.11 g of 1-{4-[(5,5-dimethyl-2,4-dioxo-3-{4-[(trifluoro-methyl)sulfonyl]phenyl}imidazolidin-1-yl)methyl]pyridin-2-yl}-3-methylurea, the characteristics of which are as follows:

1H NMR spectrum at 400 MHz: 1.44 (s, 6H); 2.73 (d, J=5.0 Hz, 3H); 4.60 (s, 2H); 6.97 (dd, J=2.0 and 5.5 Hz, 1H); 7.30 (broad s, 1H); 8.04 (d, J=9.0 Hz, 2H); 8.12 (d, J=5.5 Hz, 1H); 8.17 (broad q, J=5.0 Hz, 1H); 8.31 (d, J=9.0 Hz, 2H); 9.18 (s, 1H).

Mass Spectrum (ES): m/z=500 [M+H]⁺ base peak

### Stage b

### 1-[(2-chloropyridin-4-yl)methyl]-5,5-dimethyl-3-{4-[(trifluoromethyl)sulfonyl]phenyl}imidazolidine-2,4-dione

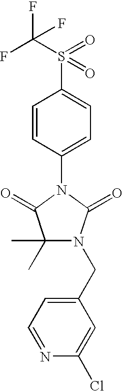

To a solution of 5 g of 3-(4-trifluoromethanesulfonyl-phenyl)-5,5-dimethylimidazolidone-2,4-dione obtained in stage a) below in 180 mL of tetrahydrofuran are successively added, under argon, 0.88 g of 60% sodium hydride and 3.61 g of 2-chloro-4-chloromethylpyridine. The solution is refluxed for 24 hours. The cooled reaction mixture is poured into distilled water and then extracted with ethyl acetate, dried over magnesium sulfate, filtered and concentrated under vacuum. The residue is purified by chromatography on a column of silica, eluting with a mixture of cyclohexane and ethyl acetate (70/30 by volume) to give 2.29 g of 1-[(2-chloropyridin-4-yl)methyl]-5,5-dimethyl-3-{4-[(trifluoromethyl)sulfonyl]phenyl}imidazolidine-2,4-dione, the characteristics of which are as follows:

1H NMR spectrum at 400 MHz: 1.44 (s, 6H); 4.68 (s, 2H); 7.49 (broad d, J=5.5 Hz, 1H); 7.42 (broad s, 1H); 8.07 (d, J=9.0 Hz, 2H); 8.31 (d, J=9.0 Hz, 2H); 8.37 (d, J=5.5 Hz, 1H).

Mass Spectrum (ES): m/z=462 [M+H]⁺ base peak

### Stage a)

### 5,5-dimethyl-3-{4-[(trifluoromethyl)sulfonyl]phenyl}-imidazolidine-2,4-dione

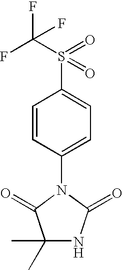

To a solution of 9.56 mL of diphosgene in toluene are successively added, under argon and at −20° C., 2.4 g of animal charcoal (3S charcoal) followed by 16.2 g of 4-tri-fluorosulfonylaniline in 150 mL of toluene and then 200 mL of toluene. The reaction mixture is refluxed for 2 hours and then cooled to room temperature. 13.26 g of 2,2-methylglycine methyl ester in 150 mL of toluene are then added, followed by 50.55 mL of triethylamine. The reaction mixture is refluxed for 15 hours, cooled to room temperature and then filtered. The organic phase is washed successively with water and with saturated sodium chloride solution, dried over magnesium sulfate, filtered and concentrated under reduced pressure. The residue is taken up in diethyl ether and the solid formed is filtered off and dried to give 14.5 g of 5,5-dimethyl-3-{4-[(trifluoromethyl)sulfonyl]phenyl}imidazolidine-2,4-dione, the characteristics of which are as follows:

1H NMR spectrum at 400 MHz: 1.44 (s, 6H); 7.99 (d, J=9.0 Hz, 2H); 8.27 (d, J=9.0 Hz, 2H); 8.81 (broad s, 1H).

Mass Spectrum (ES): m/z=337 [M+H]⁺ base peak

### EXAMPLE 5

### 1-({2-[(2,5-difluorophenyl)amino]pyridin-4-yl}-methyl)-5,5-dimethyl-3-{4-[(trifluoromethyl)thio]phenyl}imidazolidine-2,4-dione

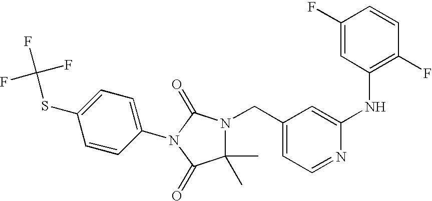

To a solution of 42.9 mg of 1-[(2-chloropyridin-4-yl)methyl]-5,5-dimethyl-3-{4-[(trifluoromethyl)thio]phenyl}imidazolidine-2,4-dione obtained in stage d) of Example 1 and 19.2 mg of 2,5-difluoroaniline in 5 mL of dioxane, under an inert atmosphere of argon, are added 2.2 mg of palladium acetate, 6.9 mg of 9,9-dimethyl-4,5-bis(diphenylphosphino)xanthene and 123 mg of caesium carbonate. The reaction medium is heated at 120° C. for 12 hours, cooled to room temperature and concentrated under reduced pressure. The residue obtained is purified by preparative HPLC chromatography (reverse-phase C18 column, eluting with a water/acetonitrile gradient containing 0.1% trifluoroacetic acid). After evaporating off the solvents under reduced pressure, 26.4 mg of 1-({2-[(2,5-difluorophenyl)amino]-pyridin-4-yl}methyl)-5,5-dimethyl-3-{4-[(trifluoromethyl)thio]-phenyl}imidazolidine-2,4-dione are obtained, the characteristics of which are as follows:

LCMS: m/Z=523.27 [M+H]⁺; RT: 1.95 min

### EXAMPLE 6

### 3,5-dichloro-N-{4-[(5,5-dimethyl-2,4-dioxo-3-{4-[(trifluoromethyl)thio]phenyl}imidazolidin-1-yl)methyl]pyridin-2-yl}benzamide

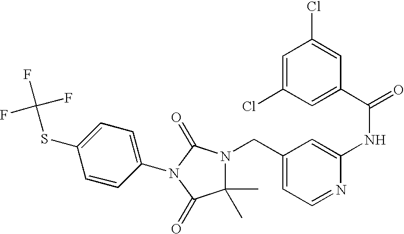

To a solution of 42.9 mg of 1-[(2-chloropyridin-4-yl)methyl]-5,5-dimethyl-3-{4-[(trifluoromethyl)thio]phenyl}imidazolidine-2,4-dione obtained in stage d) of Example 1, and 28.2 mg of 3,5-dichlorobenzamide in 5 mL of dioxane, under an inert atmosphere of argon, are added 2.2 mg of palladium acetate, 6.9 mg of 9,9-dimethyl-4,5-bis(diphenylphosphino)xanthene and 123 mg of caesium carbonate. The reaction medium is heated at 120° C. for 12 hours, cooled to room temperature and concentrated under reduced pressure. The residue obtained is purified by preparative HPLC chromatography (reverse-phase C18 column, eluting with a water/acetonitrile gradient containing 0.1% tri-fluoroacetic acid). After evaporating off the solvents under reduced pressure, 22.6 mg of 3,5-dichloro-N-{4-[(5,5-dimethyl-2,4-dioxo-3-{4-[(trifluoromethyl)thio]phenyl}imidazolidin-1-yl)methyl]pyridin-2-yl}benzamide are obtained, the characteristics of which are as follows:

LCMS: TR=2.40 min m/Z=583.30 [M+H]⁺

### EXAMPLE 7

### 2-chloro-N-{4-[(5,5-dimethyl-2,4-dioxo-3-{4-[(tri-fluoromethyl)thio]phenyl}imidazolidin-1-yl)methyl]pyridin-2-yl}-6-fluoro-3-methylbenzamide

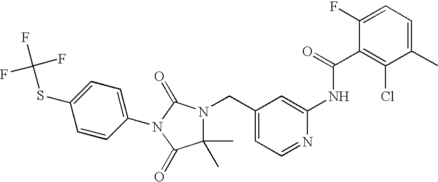

To a solution of 42.9 mg of 1-[(2-chloropyridin-4-yl)methyl]-5,5-dimethyl-3-{4-[(trifluoromethyl)thio]phenyl}imidazolidine-2,4-dione obtained in stage d) of Example 1, and 27.9 mg of 2-chloro-6-fluoro-3-methylbenzamide in 5 mL of dioxane, under an inert atmosphere of argon, are added 2.2 mg of palladium acetate, 6.9 mg of 9,9-dimethyl-4,5-bis(diphenyl-phosphino)xanthene and 123 mg of caesium carbonate. The reaction medium is heated at 120° C. for 12 hours, cooled to room temperature and concentrated under reduced pressure. The residue obtained is purified by preparative HPLC chromatography (reverse-phase C18 column, eluting with a water/acetonitrile gradient containing 0.1% trifluoroacetic acid). After evaporating off the solvents under reduced pressure, 18.2 mg of 2-chloro-N-{4-[(5,5-dimethyl-2,4-dioxo-3-{4-[(trifluoromethyl)-thio]phenyl}imidazolidin-1-yl)methyl]pyridin-2-yl}-6-fluoro-3-methylbenzamide are obtained, the characteristics of which are as follows:

LCMS: m/Z=581.31 [M+H]⁺; RT: 2.28 min

### EXAMPLE 8

### 3-({4-[(5,5-dimethyl-2,4-dioxo-3-{4-[(trifluoro-methyl)thio]phenyl}imidazolidin-1-yl)methyl]pyridin-2-yl}amino)-N,N-dimethylbenzamide

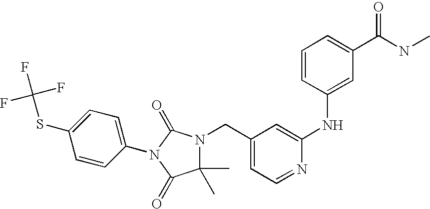

To a solution of 42.9 mg of 1-[(2-chloropyridin-4-yl)methyl]-5,5-dimethyl-3-{4-[(trifluoromethyl)thio]phenyl}imidazolidine-2,4-dione obtained in stage d) of Example 1, and 24.4 mg of 3-amino-N,N-dimethylbenzamide in 5 mL of dioxane, under an inert atmosphere of argon, are added 2.2 mg of palladium acetate, 6.9 mg of 9,9-dimethyl-4,5-bis(diphenylphosphino)xanthene and 123 mg of caesium carbonate. The reaction medium is heated at 120° C. for 12 hours, cooled to room temperature and concentrated under reduced pressure. The residue obtained is purified by preparative HPLC chromatography (reverse-phase C18 column, eluting with a water/acetonitrile gradient containing 0.1% tri-fluoroacetic acid). After evaporating off the solvents under reduced pressure, 33.2 mg of 3-({4-[(5,5-dimethyl-2,4-dioxo-3-{4-[(trifluoromethyl)thio]phenyl}imidazolidin-1-yl)methyl]pyridin-2-yl}amino)-N,N-dimethylbenzamide are obtained, the characteristics of which are as follows:

LCMS: m/Z=558.23 [M+H]⁺; RT: 1.46 min

### EXAMPLE 9

### 1-[(2-{[(1R)-2-hydroxy-1-methylethyl]amino}-pyrimidin-4-yl)methyl]-5,5-dimethyl-3-{4-[(trifluoromethyl)-sulfonyl]phenyl}imidazolidine-2,4-dione

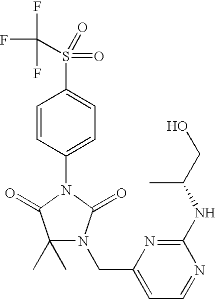

### Stage d)

### 1-[(2-{[(1R)-2-hydroxy-1-methylethyl]amino}pyrimidin-4-yl)methyl]-5,5-dimethyl-3-{4-[(trifluoromethyl)sulfonyl]-phenyl}imidazolidine-2,4-dione

A solution of 100 mg of 5,5-dimethyl-1-{[2-(methylsulfonyl)pyrimidin-4-yl]methyl}-3-{4-[(trifluoro-methyl)sulfonyl]phenyl}imidazolidine-2,4-dione obtained in stage c) below and 44.5 mg of (R) 2-amino-1-propanol in 2 mL of dioxane is poured into a tube and sealed with a Teflon septum. The tube is placed in a microwave oven (Emrys Optimizer, Personal Chemistry) and the solution is agitated at 120° C. for 1 hour. After cooling to room temperature, the solvent is evaporated off under reduced pressure and the residue is purified by preparative HPLC chromatography (reverse-phase C18 column, eluting with a water/acetonitrile gradient containing 0.1% trifluoroacetic acid). After freeze-drying the solution, a white solid is obtained, which is treated with saturated sodium hydrogen carbonate solution and extracted with ethyl acetate. The organic phase is dried over anhydrous sodium sulfate and evaporated to give 49.7 mg of 1-[(2-{[(1R)-2-hydroxy-1-methylethyl]amino}pyrimidin-4-yl)methyl]-5,5-dimethyl-3-{4-[(trifluoromethyl)sulfonyl]phenyl}imidazolidine-2,4-dione, the characteristics of which are as follows:

1H NMR spectrum at 400 MHz: d=8.32 (d, 2H); 8.22 (d, 1H); 8.05 (d, 2H); 6.80 (d, 1H); 6.67 (d, 1H); 4.65 (t, 1H); 4.46 (s, 2H); 3.93 (m, 1H); 3.44 (m, 1H); 1.48 (s, 6H); 1.09 (s, 3H)

Mass Spectrum (ES): m/z=502 [M+H]⁺

### Stage c)

### 5,5-dimethyl-1-{[2-(methylsulfonyl)pyrimidin-4-yl]-methyl}-3-{4-[(trifluoromethyl)sulfonyl]phenyl}imidazolidine-2,4-dione

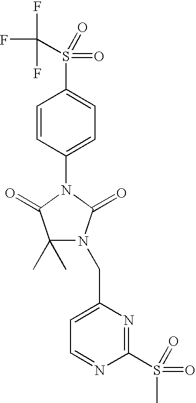

To a solution of 4.90 g of 5,5-dimethyl-1-{[2-(methyl-thio)pyrimidin-4-yl]methyl}-3-{4-[(trifluoromethyl)thio]-phenyl}imidazolidine-2,4-dione obtained in stage b) below in 80 mL of dichloroethane are added 16.37 g of 3-chloroperbenzoic acid (70%). The reaction mixture is stirred for 16 hours at room temperature and a further 2.73 g of 3-chloroperbenzoic acid (70%) are added, and the reaction mixture is heated at 40° C. for 2 hours. The solution is then washed twice with saturated sodium hydrogen carbonate solution. The organic phase is dried over anhydrous sodium sulfate and filtered, and the solvent is evaporated off under reduced pressure. The residue is purified by chromatography on a column of silica, eluting with a gradient of heptane and ethyl acetate to give 4.30 g of 5,5-dimethyl-1-{[2-(methylsulfonyl)pyrimidin-4-yl]methyl}-3-{4-[(trifluoromethyl)sulfonyl]phenyl}imidazolidine-2,4-dione, the characteristics of which are as follows:

1H NMR spectrum at 400 MHz: d=9.06 (d, 1H); 8.32 (d, 2H); 8.03 (m, 3H); 4.89 (s, 2H); 3.43 (s, 3H); 1.51 (s, 6H)

Mass Spectrum (ES): m/z=508 [M+H]⁺

### Stage b)

### 5,5-dimethyl-1-{[2-(methylthio)pyrimidin-4-yl]-methyl}-3-{4-[(trifluoromethyl)thio]phenyl}imidazolidine-2,4-dione

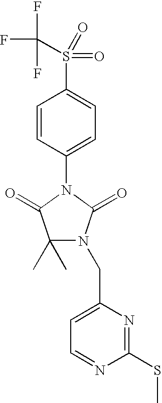

To a solution of 1.00 g of 5,5-dimethyl-3-{4-[(trifluoro-methyl)thio]phenyl}imidazolidine-2,4-dione obtained in stage c) of Example 1 in 10 mL of N,N-dimethylformamide are added 0.087 g of sodium hydride at 0° C. After stirring for 10 minutes, 2.88 g of a 40% solution of 4-bromomethyl-2-methylthiopyrimidine in hexane are added and the mixture is stirred for 4 hours at room temperature. The solvent is then evaporated off under reduced pressure and the residue is purified by preparative HPLC (reverse-phase C18 column, eluting with a water/acetonitrile gradient containing 0.1% tri-fluoroacetic acid). After freeze-drying the fractions, 1.12 g of 5,5-dimethyl-1-{[2-(methylthio)pyrimidin-4-yl]methyl}-3-{4-[(trifluoromethyl)thio]phenyl}imidazolidine-2,4-dione are obtained, the characteristics of which are as follows:

1H NMR spectrum at 400 MHz: 8.59 (d, 1H); 7.86 (d, 2H); 7.65 (d, 2H); 7.32 (d, 1H); 4.65 (s, 2H); 2.55-2.45 (s, 3H); 1.45 (s, 6H)

Mass Spectrum (ES): m/z=443 [M+H]⁺

Examples 10 to 17, the names and structures of which are described below, are prepared as indicated above in the General Schemes.

| Structure | Name |
|---|---|
| 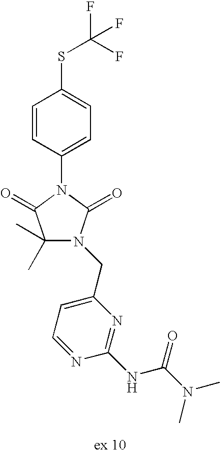 | 3-{4-[(5,5-dimethyl-2,4-dioxo-3-{4-[(trifluoro-methyl)thio]phenyl}imidazolidin-1-yl)methyl]-pyrimidin-2-yl}-1,1-dimethylurea |
| 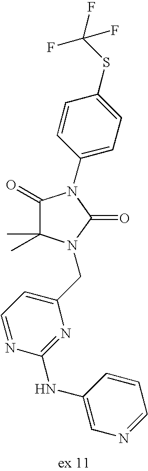 | 5,5-dimethyl-1-{[2-(pyridin-3-ylamino)pyrimidin-4-yl]methyl}-3-{4-[(trifluoromethyl)thio]phenyl}-imidazolidine-2,4-dione |
| 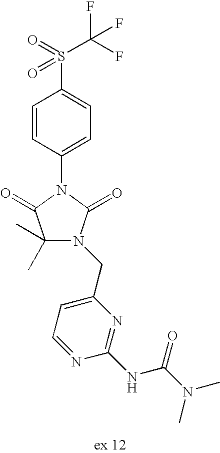 | 3-{4-[(5,5-dimethyl-2,4-dioxo-3-{4-[(trifluoro-methyl)sulfonyl]phenyl}imidazolidin-1-yl)-methyl]pyrimidin-2-yl}-1,1-dimethylurea |
| 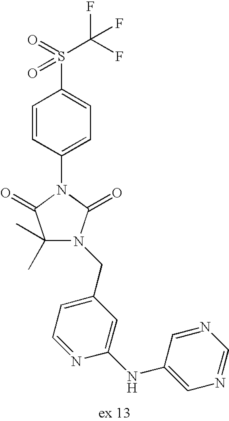 | 5,5-dimethyl-1-{[2-(pyrimidin-5-ylamino)pyridin-4-yl]methyl}-3-{4-[(trifluoromethyl)sulfonyl]-phenyl}imidazolidine-2,4-dione |
| 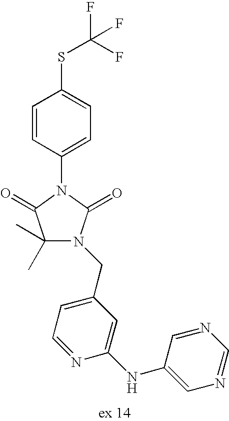 | 5,5-dimethyl-1-{[2-(pyrimidin-5-ylamino)pyridin-4-yl]methyl}-3-{4-[(trifluoromethyl)thio]phenyl}-imidazolidine-2,4-dione |
| 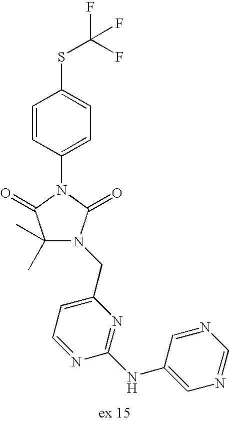 | 5,5-dimethyl-1-{[2-(pyrimidin-5-ylamino)-pyrimidin-4-yl]methyl}-3-{4-[(trifluoromethyl)-thio]phenyl}imidazolidine-2,4-dione |
| 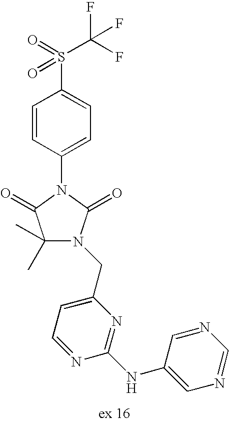 | 5,5-dimethyl-1-{[2-(pyrimidin-5-ylamino)-pyrimidin-4-yl]methyl}-3-{4-[(trifluoromethyl)-sulfonyl]phenyl}imidazolidine-2,4-dione |
| 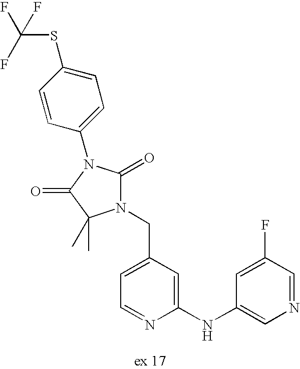 | 1-({2-[(5-fluoropyridin-3-yl)amino]pyridin-4-yl}-methyl)-5,5-dimethyl-3-{4-[(trifluoromethyl)-thio]phenyl}imidazolidine-2,4-dione |

### EXAMPLE 10

### 3-{4-[(5,5-dimethyl-2,4-dioxo-3-{4-[(trifluoro-methyl)thio]phenyl}imidazolidin-1-yl)methyl]pyrimidin-2-yl}-1,1-dimethylurea

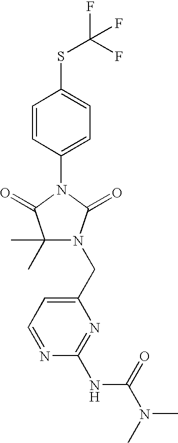

### Stage i)

### 3-{4-[(5,5-dimethyl-2,4-dioxo-3-{4-[(trifluoro-methyl)thio]phenyl}imidazolidin-1-yl)methyl]pyrimidin-2-yl}-1,1-dimethylurea

To a solution of 90 mg of phenyl {4-[(5,5-dimethyl-2,4-dioxo-3-{4-[(trifluoromethyl)thio]phenyl}imidazolidin-1-yl)methyl]-pyrimidin-2-yl}carbamate obtained in stage h) below in 4 mL of tetrahydrofuran are added 0.85 mL of a 2M solution of dimethylamine in tetrahydrofuran under argon. The reaction mixture is stirred for 15 hours at room temperature and concentrated under reduced pressure. The residue is purified by chromatography on a column of silica, eluting with a mixture of dichloromethane and methanol (98/2 by volume) to give 30 mg of 3-{4-[(5,5-dimethyl-2,4-dioxo-3-{4-[(trifluoromethyl)thio]-phenyl}imidazolidin-1-yl)methyl]pyrimidin-2-yl}-1,1-dimethyl-urea, the characteristics of which are as follows:

1H NMR spectrum at 400 MHz: 1.45 (s, 6H); 2.90 (s, 6H); 4.57 (s, 2H); 7.09 (d, J=5.5 Hz, 1H); 7.67 (d, J=8.5 Hz, 2H); 7.86 (d, J=8.5 Hz, 2H); 8.48 (d, J=5.5 Hz, 1H); 9.25 (s, 1H).

Mass Spectrum (ES): m/z=483 [M+H]⁺

### Stage h)

### phenyl {4-[(5,5-dimethyl-2,4-dioxo-3-{4-[(trifluoro-methyl)thio]phenyl}imidazolidin-1-yl)methyl]pyrimidin-2-yl}carbamate

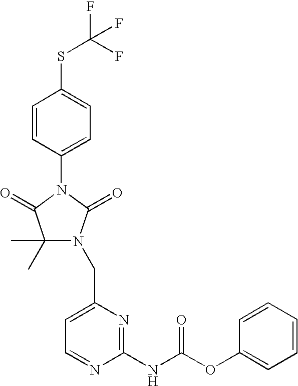

To a solution of 0.8 g of 1-[(2-aminopyrimidin-4-yl)methyl]-5,5-dimethyl-3-{4-[(trifluoromethyl)thio]phenyl}imidazolidine-2,4-dione obtained in stage g) below in 40 mL of tetrahydrofuran are added successively, at 0° C. under argon, 0.257 mL of pyridine and 0.34 mL of phenyl chloroformate and the solution is then stirred for 15 hours at room temperature. The reaction mixture is taken up in ethyl acetate, washed successively with concentrated hydrochloric acid, with water, with saturated sodium hydrogen carbonate solution and with saturated sodium chloride solution, and dried over magnesium sulfate. After filtration, the solution is concentrated under reduced pressure and the residue is purified by chromatography on a column of silica, eluting with a mixture of ethyl acetate and cyclohexane (65/35 by volume) to give 0.68 g of phenyl {4-[(5,5-dimethyl-2,4-dioxo-3-{4-[(trifluoromethyl)thio]phenyl}-imidazolidin-1-yl)methyl]pyrimidin-2-yl}carbamate, the characteristics of which are as follows:

1H NMR spectrum at 400 MHz: 1.46 (s, 6H); 4.67 (s, 2H); 6.75 (m, 3H); from 7.02 to 7.50 (m, 3H); 7.60 (d, J=8.5 Hz, 2H); 7.82 (d, J=8.5 Hz, 2H); 8.61 (d, J=5.5 Hz, 1H); 9.30 (s, 1H).

Mass Spectrum (ES): m/z=532 [M+H]⁺

### Stage g)

### 1-[(2-aminopyrimidin-4-yl)methyl]-5,5-dimethyl-3-{4-[(trifluoromethyl)thio]phenyl}imidazolidine-2,4-dione

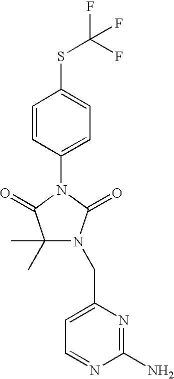

To a solution of 0.49 g of 5,5-dimethyl-1-{[2-(methylsulfonyl)pyrimidin-4-yl]methyl}-3-{4-[(trifluoromethyl)-thio]phenyl}imidazolidine-2,4-dione obtained in stage f) below in 2.2 mL of dioxane are added 2.2 mL of concentrated aqueous ammonia. The reaction mixture is heated by microwave at 120° C. for 1 hour, left at room temperature for 15 hours and then concentrated under reduced pressure. The residue is purified by chromatography on a column of silica, eluting with a mixture of ethyl acetate and cyclohexane (75/25 by volume) to give 0.31 g of 1-[(2-aminopyrimidin-4-yl)methyl]-5,5-dimethyl-3-{4-[(tri-fluoromethyl)thio]phenyl}imidazolidine-2,4-dione, the characteristics of which are as follows:

1H NMR spectrum at 400 MHz: 1.43 (s, 6H); 4.41 (s, 2H); 6.59 (s, 2H); 6.66 (d, J=5.5 Hz, 1H); 7.66 (d, J=8.5 Hz, 2H); 7.86 (d, J=8.5 Hz, 2H); 8.19 (d, J=5.5 Hz, 1H).

Mass Spectrum (1E): m/z=411: [M]⁺ (base peak)
m/z=396: [M]+—CH3
m/z=303: [M]+—C6H6N3
m/z=109: [C5H6N3]+

### Stage f)

### 5,5-dimethyl-1-{[2-(methylsulfonyl)pyrimidin-4-yl]-methyl}-3-{4-[(trifluoromethyl)thio]phenyl}imidazolidine-2,4-dione

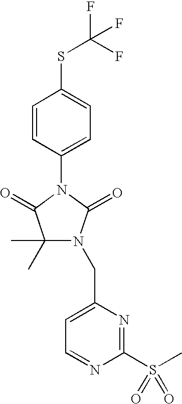

1.32 g of 5,5-dimethyl-3-{4-[(trifluoromethyl)thio]phenyl}imidazolidine-2,4-dione obtained in stage c) of Example 1 are added under argon to a suspension of 0.26 g of sodium hydride in 30 mL of dimethylformamide. After stirring at room temperature for 1.5 hours, a solution of 1.35 g of 4-(chloromethyl)-2-(methylsulfonyl)pyrimidine obtained in stage e) below in 5 mL of dimethylformamide is added. The reaction mixture is stirred for 15 hours at room temperature and then poured into distilled water and extracted with ethyl acetate. The aqueous phase is washed successively with water and with saturated sodium chloride solution, dried over magnesium sulfate, filtered and concentrated under reduced pressure. The residue is purified by chromatography on a column of silica, eluting with a mixture of ethyl acetate and cyclohexane (65/35 by volume) to give 0.35 g of 5,5-dimethyl-1-{[2-(methylsulfonyl)pyrimidin-4-yl]methyl}-3-{4-[(trifluoromethyl)thio]phenyl}imidazolidine-2,4-dione, the characteristics of which are as follows:

1H NMR spectrum at 400 MHz: 1.49 (s, 6H); 3.41 (s, 3H); 4.88 (s, 2H); 7.66 (d, J=8.5 Hz, 2H); 7.86 (d, J=8.5 Hz, 2H); 7.99 (d, J=5.5 Hz, 1H); 9.04 (d, J=5.5 Hz, 1H).

Mass Spectrum (ES): m/z=475 [M+H]⁺
m/z=473 [M−H]⁻

### Stage e)

### 4-(chloromethyl)-2-(methylsulfonyl)pyrimidine

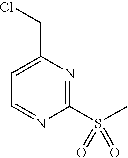

To a solution of 1.2 g of [2-(methylsulfonyl)pyrimidin-4-yl]-methanol obtained in stage d) below in 28 mL of dichloromethane are added successively 2.28 mL of dimethylformamide and 0.56 mL of thionyl chloride. The reaction mixture is stirred at room temperature for 2 hours and then concentrated under reduced pressure to give 1.3 g of 4-(chloromethyl)-2-(methylsulfonyl)-pyrimidine, the characteristics of which are as follows:

1H NMR spectrum at 400 MHz: 3.42 (s, 3H); 4.95 (s, 2H); 7.99 (d, J=5.5 Hz, 1H); 9.12 (d, J=5.5 Hz, 1H)

Mass Spectrum (1E): m/z=206: [M]⁺
m/z=191: [M]⁺—CH3
m/z=142: [M]⁺—SO2
m/z=127: [M]⁺—SO2CH3 (base peak)

### Stage d)

### [2-(methylsulfonyl)pyrimidin-4-yl]methanol

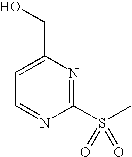

To a solution of 2.66 g of 2-(methylsulfonyl)-4-[(tetrahydro-2H-pyran-2-yloxy)methyl]pyrimidine obtained in stage c) below in 244 mL of ethanol are added 0.8 mL of concentrated hydrochloric acid. The reaction mixture is stirred at room temperature for 1 hour and then concentrated under reduced pressure to give 1.2 g of [2-(methylsulfonyl)pyrimidin-4-yl]-methanol, the characteristics of which are as follows:

1H NMR spectrum at 300 MHz: 3.40 (s, 3H); 4.68 (d, J=5.5 Hz, 2H); 5.87 (t, J=5.5 Hz, 1H); 7.85 (d, J=5.5 Hz, 1H); 9.02 (d, J=5.5 Hz, 1H).

Mass Spectrum (1E): m/z=188: [M]⁺
m/z=158: [M]⁺—CH2O
m/z=124: [M]⁺—SO2
m/z=109: [M]⁺—SO2CH3 (base peak)

### Stage c)

### 2-(methylsulfonyl)-4-[(tetrahydro-2H-pyran-2-yloxy)-methyl]pyrimidine

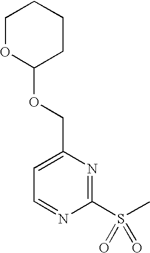

To a solution of 2.63 g of 2-(methylthio)-4-[(tetrahydro-2H-pyran-2-yloxy)methyl]pyrimidine obtained in stage b) below in 79 mL of dichloromethane and 8.8 mL of methanol are added 8.3 g of meta-chloroperbenzoic acid. The reaction mixture is stirred at room temperature for 5 hours. The organic phase is then washed successively with saturated sodium bisulfite solution, with saturated sodium bicarbonate solution and with saturated sodium chloride solution, dried over magnesium sulfate and filtered. The solvent is then distilled off under reduced pressure to give 3.02 g of 2-(methylsulfonyl)-4-[(tetrahydro-2H-pyran-2-yloxy)methyl]pyrimidine, the characteristics of which are as follows:

1H NMR spectrum at 400 MHz: from 1.40 to 1.87 (m, 6H); 3.40 (s, 3H); 3.49 (m, 1H); 3.79 (m, 1H); 4.74 (d, J=16.0 Hz, 1H); 4.80 (t, J=3.0 Hz, 1H); 4.83 (d, J=16.0 Hz, 1H); 7.86 (d, J=5.5 Hz, 1H); 9.05 (d, J=5.5 Hz, 1H).

Mass Spectrum (ES): m/z=273 [M+H]⁺ (base peak)
m/z=189 [M+H]⁺ —C5H9O (base peak)

### Stage b)

### 2-(methylthio)-4-[(tetrahydro-2H-pyran-2-yloxy)-methyl]pyrimidine

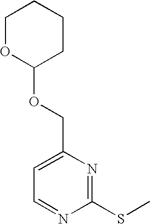

To a solution of 3.4 g of [2-(methylthio)pyrimidin-4-yl]-methanol obtained in stage a) below in 60 mL of dichloromethane are added 2.197 g of 3,4-dihydropyran and 0.414 g of para-toluenesulfonic acid. The reaction mixture is stirred at room temperature for 15 hours and then refluxed for 1 hour and cooled in an ice bath. The organic phase is then washed successively with saturated sodium bicarbonate solution, with water and with saturated sodium chloride solution, dried over magnesium sulfate, filtered and concentrated under reduced pressure. The residue is purified by chromatography on a column of silica, eluting with a mixture of ethyl acetate and cyclohexane (10/90 by volume) to give 4.75 g of 2-(methylthio)-4-[(tetrahydro-2H-pyran-2-yloxy)methyl]pyrimidine, the characteristics of which are as follows:

1H NMR spectrum at 300 MHz: from 1.40 to 1.87 (m, 6H); 2.50 (masked s, 3H); 3.48 (m, 1H); 3.78 (m, 1H); 4.51 (d, J=15.0 Hz, 1H); 4.65 (d, J=15.0 Hz, 1H); 4.74 (t, J=3.0 Hz, 1H); 7.23 (d, J=5.5 Hz, 1H); 8.61 (d, J=5.5 Hz, 1H).

Mass Spectrum (1E): m/z=240; [M]⁺
m/z=140: [M]⁺—C5H9O2

Mass Spectrum (IC): m/z=241 [M+H]⁺

### Stage a)

### [2-(methylthio)pyrimidin-4-yl]methanol

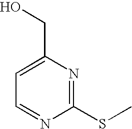

To a solution of 10 g of 4-formyl-2-(methylthio)pyrimidine in 200 mL of methanol are added portionwise, under argon, 4.9 g of sodium borohydride. The reaction mixture is stirred at room temperature for 15 hours and then concentrated under reduced pressure. The residue is taken up in dichloromethane, washed successively with water and with saturated sodium chloride solution, dried over magnesium sulfate, filtered and concentrated under reduced pressure. The residue is triturated from diisopropyl ether to give 5.4 g of [2-(methylthio)-pyrimidin-4-yl]methanol, the characteristics of which are as follows:

1H NMR spectrum at 300 MHz: 2.49 (s, 3H); 4.49 (d, J=5.5 Hz, 2H); 5.60 (t, J=5.5 Hz, 1H); 7.24 (d, J=5.5 Hz, 1H); 8.60 (d, J=5.5 Hz, 1H).

Mass Spectrum (1E): m/z=156: [M]⁺
m/z=138: [M]⁺—H2O

### EXAMPLE 11

### 5,5-dimethyl-1-{[2-(pyridin-3-ylamino)pyrimidin-4-yl]methyl}-3-{4-[(trifluoromethyl)thio]phenyl}imidazolidine-2,4-dione

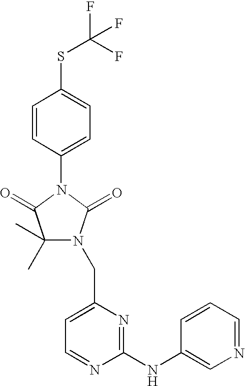

To a solution of 0.13 g of 1-[(2-aminopyrimidin-4-yl)methyl]-5,5-dimethyl-3-{4-[(trifluoromethyl)thio]phenyl}imidazolidine-2,4-dione obtained in stage g) of Example 10 in 10 mL of dioxane, are successively added, under argon, 0.1 g of 3-bromopyridine, 0.39 g of caesium carbonate, 0.044 g of 9,9-dimethyl-4,5-bis(diphenylphosphino)xanthene (Xantphos) and 0.015 g of palladium acetate. The reaction mixture is heated at 100° C. for 15 hours and then concentrated under reduced pressure. The residue is purified by chromatography on a column of silica, eluting with a mixture of dichloromethane, acetonitrile and methanol (98/1/1 by volume) to give 0.0264 g of 5,5-dimethyl-1-{[2-(pyridin-3-ylamino)pyrimidin-4-yl]-methyl}-3-{4-[(trifluoromethyl)thio]phenyl}imidazolidine-2,4-dione, the characteristics of which are as follows:

1H NMR spectrum at 400 MHz: 1.45 (s, 6H); 4.61 (s, 2H); 7.00 (d, J=5.5 Hz, 1H); 7.21 (dd, J=5.0 and 8.0 Hz, 1H); 7.70 (d, J=8.5 Hz, 2H); 7.87 (d, J=8.5 Hz, 2H); 8.14 (broad d, J=5.5 Hz, 1H); 8.19 (broad d, J=8.0 Hz, 1H); 8.48 (d, J=5.5 Hz, 1H); 8.92 (broad d, J=5.0 Hz, 1H); 9.80 (s, 1H).

Mass Spectrum (ES): m/z=489 [M+H]⁺
m/z=487 [M−H]⁻

### EXAMPLE 12

### 3-{4-[(5,5-dimethyl-2,4-dioxo-3-{4-[(trifluoro-methyl)sulfonyl]phenyl}imidazolidin-1-yl)methyl]pyrimidin-2-yl}-1,1-dimethylurea

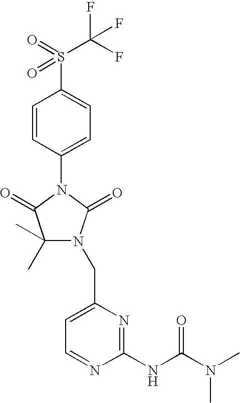

### Stage c)

### 3-{4-[(5,5-dimethyl-2,4-dioxo-3-{4-[(trifluoro-methyl)sulfonyl]phenyl}imidazolidin-1-yl)methyl]pyrimidin-2-yl}-1,1-dimethylurea

To a solution of 0.12 g of phenyl {4-[(5,5-dimethyl-2,4-dioxo-3-{4-[(trifluoromethyl)sulfonyl]phenyl}imidazolidin-1-yl)-methyl]pyrimidin-2-yl}carbamate obtained in stage b) below in 4 mL of tetrahydrofuran are added 1.06 mL of a 2M solution of dimethylamine in tetrahydrofuran. The reaction mixture is stirred at room temperature under argon for three hours and then concentrated under reduced pressure. The residue is purified by chromatography on a column of silica, eluting with a mixture of dichloromethane and methanol (98/2 by volume) to give 0.06 g of 3-{4-[(5,5-dimethyl-2,4-dioxo-3-{4-[(trifluoro-methyl)sulfonyl]phenyl}imidazolidin-1-yl)methyl]pyrimidin-2-yl}-1,1-dimethylurea, the characteristics of which are as follows:

1H NMR spectrum at 400 MHz: 1.48 (s, 6H); 2.89 (s, 6H); 4.59 (s, 2H); 7.11 (d, J=5.5 Hz, 1H); 8.03 (d, J=8.5 Hz, 2H); 8.30 (d, J=8.5 Hz, 2H); 8.48 (d, J=5.5 Hz, 1H); 9.27 (s, 1H).

Mass Spectrum (ES): m/z=515 [M+H]⁺
m/z=513 [M−H]⁻

### Stage b)

### phenyl {4-[(5,5-dimethyl-2,4-dioxo-3-{4-[(trifluoro-methyl)sulfonyl]phenyl}imidazolidin-1-yl)methyl]pyrimidin-2-yl}carbamate

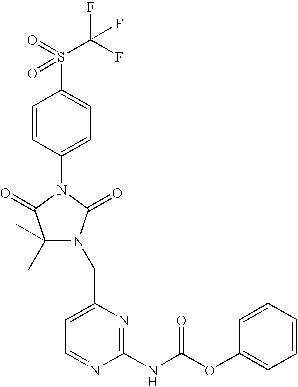

To a solution of 0.8 g of 1-[(2-aminopyrimidin-4-yl)methyl]-5,5-dimethyl-3-{4-[(trifluoromethyl)sulfonyl]phenyl}imidazolidine-2,4-dione obtained in stage a) below in 40 mL of tetrahydrofuran are added successively, at 0° C. under argon, 0.184 mL of pyridine and 0.23 mL of phenyl chloroformate and the solution is then stirred for 15 hours at room temperature. The reaction mixture is taken up in ethyl acetate and washed successively with concentrated hydrochloric acid, with water, with saturated sodium hydrogen carbonate solution and with saturated sodium chloride solution, and dried over magnesium sulfate. After filtration, the solution is concentrated under reduced pressure and the residue is purified by chromatography on a column of silica, eluting with a mixture of ethyl acetate and cyclohexane (65/35 by volume) to give 0.68 g of phenyl {4-[(5,5-dimethyl-2,4-dioxo-3-{4-[(trifluoromethyl)sulfonyl]-phenyl}imidazolidin-1-yl)methyl]pyrimidin-2-yl}carbamate, the characteristics of which are as follows:

1H NMR spectrum at 400 MHz: 1.43 (s, 6H); 4.55 (s, 2H); 7.01 (d, J=5.5 Hz, 1H); 7.08 (d, J=7.5 Hz, 2H); 7.28 (partially masked t, J=7.5 Hz, 1H); 7.31 (t, J=7.5 Hz, 2H); 7.84 (s, 1H); 7.86 (d, J=8.5 Hz, 2H); 7.97 (d, J=8.5 Hz, 2H); 8.50 (d, J=5.5 Hz, 1H).

Mass Spectrum (ES): m/z=563 [M+H]⁺

### Stage a)

### 1-[(2-aminopyrimidin-4-yl)methyl]-5,5-dimethyl-3-{4-[(trifluoromethyl)sulfonyl]phenyl}imidazolidine-2,4-dione

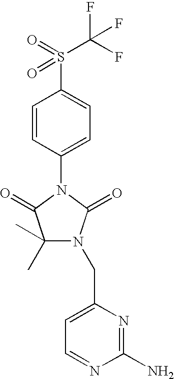

To a solution of 0.91 g of 1-(2-methanesulfonylpyrimidin-4-yl-methyl)-5,5-dimethyl-3-(4-trifluoromethylsulfonyl-phenyl)imidazolidine-2,4-dione obtained in stage c) of Example 9 in 5 mL of dioxane are added 5 mL of concentrated aqueous ammonia. The reaction mixture is heated by microwave at 120° C. for 1 hour, left at room temperature for 15 hours and then concentrated under reduced pressure. The residue is purified by chromatography on a column of silica, eluting with a mixture of ethyl acetate and cyclohexane (70/30 by volume) to give 0.54 g of 1-[(2-aminopyrimidin-4-yl)methyl]-5,5-dimethyl-3-{4-[(tri-fluoromethyl)sulfonyl]phenyl}imidazolidine-2,4-dione, the characteristics of which are as follows:

1H NMR spectrum at 400 MHz: 1.46 (s, 6H); 4.43 (s, 2H); 6.59 (broad s, 2H); 6.69 (d, J=5.5 Hz, 1H); 8.03 (d, J=8.5 Hz, 2H); 8.19 (d, J=5.5 Hz, 1H); 8.30 (d, J=8.5 Hz, 2H).

Mass Spectrum (ES): m/z=444 [M+H]⁺

### EXAMPLE 13

### 5,5-dimethyl-1-{[2-(pyrimidin-5-ylamino)pyridin-4-yl]methyl}-3-{4-[(trifluoromethyl)sulfonyl]phenyl}imidazolidine-2,4-dione

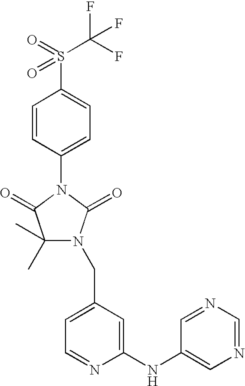

### Stage c)

### 5,5-dimethyl-1-{[2-(pyrimidin-5-ylamino)pyridin-4-yl]methyl}-3-{4-[(trifluoromethyl)sulfonyl]phenyl}imidazolidine-2,4-dione

To a solution of 0.36 g of 1-[(2-aminopyridine-4-yl)methyl]-5,5-dimethyl-3-{4-[(trifluoromethyl)sulfonyl]phenyl}imidazolidine-2,4-dione obtained in stage b) below in 20 mL of dioxane are successively added, under argon, 0.19 g of 5-bromopyrimidine, 0.056 g of 9,9-dimethyl-4,5-bis(diphenylphosphino)xanthene (Xantphos), 0.027 g of palladium acetate and 1 g of caesium carbonate. The reaction mixture is heated at 90° C. for 3 hours and then filtered and concentrated under reduced pressure. The residue is purified by chromatography on a column of silica, eluting with a mixture of dichloromethane and methanol (98/2 by volume) to give 0.15 g of 5,5-dimethyl-1-{[2-(pyrimidin-5-ylamino)pyridin-4-yl]methyl}-3-{4-[(trifluoromethyl)sulfonyl]-phenyl}imidazolidine-2,4-dione, the characteristics of which are as follows:

1H NMR spectrum at 400 MHz: 1.48 (s, 6H); 4.61 (s, 2H); 6.90 (broad s, 1H); 6.94 (broad d, J=5.5 Hz, 1H); 8.07 (d, J=8.5 Hz, 2H); 8.19 (d, J=5.5 Hz, 1H); 8.31 (d, J=8.5 Hz, 2H); 8.70 (s, 1H); 9.12 (s, 2H); 9.38 (s, 1H).

Mass Spectrum (ES): m/z=521 [M+H]⁺
m/z=519 [M−H]⁻

### Stage b)

### 1-[(2-aminopyridin-4-yl)methyl]-5,5-dimethyl-3-{4-[(trifluoromethyl)sulfonyl]phenyl}imidazolidine-2,4-dione

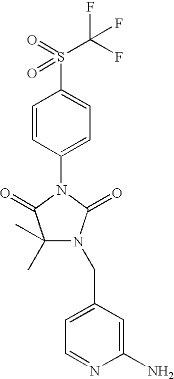

To a solution of 1.5 g of N-{4-[(5,5-dimethyl-2,4-dioxo-3-{4-[(trifluoromethyl)sulfonyl]phenyl}imidazolidin-1-yl)methyl]-pyridin-2-yl}acetamide obtained in stage a) below in 25 mL of methanol are added 0.62 mL of a 30% solution of sodium hydroxide in water. The reaction mixture is heated at 50° C. for 24 hours and then concentrated under reduced pressure. The residue is purified by chromatography on a column of silica, eluting with a mixture of ethyl acetate and cyclohexane (85/15 by volume) to give 0.4 g of 1-[(2-aminopyridin-4-yl)methyl]-5,5-dimethyl-3-{4-[(trifluoromethyl)sulfonyl]phenyl}imidazolidine-2,4-dione, the characteristics of which are as follows:

1H NMR spectrum at 400 MHz: 1.50 (s, 6H); 3.41 (s, 3H); 4.89 (s, 2H); 8.00 (d, J=7.0 Hz, 1H); 8.03 (d, J=8.5 Hz, 2H); 8.30 (d, J=8.5 Hz, 2H); 9.05 (d, J=7.0 Hz, 1H).

Mass Spectrum (ES): m/z=443 [M+H]⁺

### Stage a)

### N-{4-[(5,5-dimethyl-2,4-dioxo-3-{4-[(trifluoro-methyl)sulfonyl]phenyl}imidazolidin-1-yl)methyl]pyridin-2-yl}acetamide

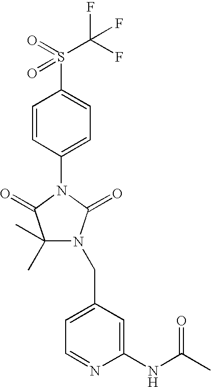

To a solution of 3 g of 1-(2-Chloropyridin-4-ylmethyl)-5,5-dimethyl-3-(4-trifluoromethanesulfonylphenyl)imidazolidine-2,4-dione obtained in stage b) of Example 4 in 60 mL of dioxane are successively added, under argon, 0.96 g of acetamide, 0.45 g of 9,9-dimethyl-4,5-bis(diphenylphosphino)xanthene (Xantphos), 0.146 g of palladium acetate and 7.4 g of caesium carbonate. The reaction mixture is refluxed for 5 hours and then filtered and concentrated under reduced pressure. The residue is purified by chromatography on a column of silica, eluting with a mixture of ethyl acetate and cyclohexane (60/40 by volume) to give 1.5 g of N-{4-[(5,5-dimethyl-2,4-dioxo-3-{4-[(trifluoro-methyl)sulfonyl]phenyl}imidazolidin-1-yl)methyl]pyridin-2-yl}-acetamide, the characteristics of which are as follows:

1H NMR spectrum at 400 MHz: 1.43 (s, 6H); 2.07 (s, 3H); 4.63 (s, 2H); 7.15 (dd, J=1.5 and 5.5 Hz, 1H); 8.03 (d, J=8.5 Hz, 2H); 8.11 (broad s, 1H); 8.24 (d, J=5.5 Hz, 1H); 8.30 (d, J=8.5 Hz, 2H); 10.5 (broad s, 1H).

Mass Spectrum (ES): m/z=485 [M+H]⁺
m/z=483 [M−H]⁻

### EXAMPLE 14

### 5,5-dimethyl-1-{[2-(pyrimidin-5-ylamino)pyridin-4-yl]methyl}-3-{4-[(trifluoromethyl)thio]phenyl}imidazolidine-2,4-dione

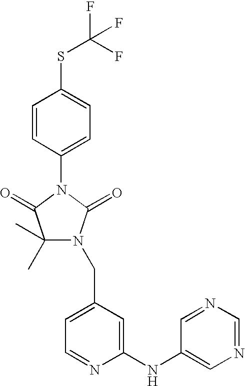

### Stage c)

### 5,5-dimethyl-1-{[2-(pyrimidin-5-ylamino)pyridin-4-yl]methyl}-3-{4-[(trifluoromethyl)thio]phenyl}imidazolidine-2,4-dione

To a solution of 0.2 g of 1-[(2-aminopyridin-4-yl)methyl]-5,5-dimethyl-3-{4-[(trifluoromethyl)thio]phenyl}imidazolidine-2,4-dione obtained in stage b) below in 5 mL of dioxane are successively added, under argon, 0.1 g of 5-bromopyrimidine, 0.025 g of 9,9-dimethyl-4,5-bis(diphenylphosphino)xanthene (Xantphos), 0.01 g of palladium acetate and 0.55 g of caesium carbonate. The reaction mixture is refluxed for 15 hours and then filtered and concentrated under reduced pressure. The residue is purified by chromatography on a column of silica, eluting with a mixture of dichloromethane, methanol and aqueous ammonia (93/6/1 by volume) to give 0.02 g of 5,5-dimethyl-1-{[2-(pyrimidin-5-ylamino)pyridin-4-yl]methyl}-3-{4-[(trifluoro-methyl)thio]phenyl}imidazolidine-2,4-dione, the characteristics of which are as follows:

1H NMR spectrum at 300 MHz: 1.45 (s, 6H); 4.59 (s, 2H); 6.90 (broad s, 1H); 6.92 (broad d, J=5.5 Hz, 1H); 7.69 (d, J=8.5 Hz, 2H); 7.87 (d, J=8.5 Hz, 2H); 8.18 (d, J=5.5 Hz, 1H); 8.70 (s, 1H); 9.13 (s, 2H); 9.40 (s, 1H).

Mass Spectrum (ES): m/z=489 [M+H]⁺
m/z=487 [M−H]⁻

### Stage b)

### 1-[(2-aminopyridin-4-yl)methyl]-5,5-dimethyl-3-{4-[(trifluoromethyl)thio]phenyl}imidazolidine-2,4-dione

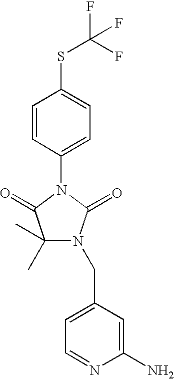

To a solution of 1.54 g of N-{4-[(5,5-dimethyl-2,4-dioxo-3-{4-[(trifluoromethyl)thio]phenyl}imidazolidin-1-yl)methyl]pyridin-2-yl}acetamide obtained in stage a) below in 25 mL of methanol is added 0.68 mL of a 30% solution of sodium hydroxide in water. The reaction mixture is heated at 50° C. for 8 hours and then concentrated under reduced pressure. The residue is purified by chromatography on a column of silica, eluting with a mixture of dichloromethane and methanol (98/2 by volume) to give 0.77 g of 1-[(2-aminopyridin-4-yl)methyl]-5,5-dimethyl-3-{4-[(trifluoromethyl)thio]phenyl}imidazolidine-2,4-dione, the characteristics of which are as follows:

1H NMR spectrum at 400 MHz: 1.40 (s, 6H); 4.44 (s, 2H); 5.88 (broad s, 2H); 6.42 (broad s, 1H); 6.50 (dd, J=1.5 and 5.5 Hz, 1H); 7.67 (d, J=8.5 Hz, 2H); 7.83 (d, J=5.5 Hz, 1H); 7.88 (d, J=8.5 Hz, 2H).

Mass Spectrum (ES): m/z=411 [M+H]⁺

### Stage a)

### N-{4-[(5,5-dimethyl-2,4-dioxo-3-{4-[(trifluoromethyl)thio]phenyl}imidazolidin-1-yl)methyl]pyridin-2-yl}-acetamide

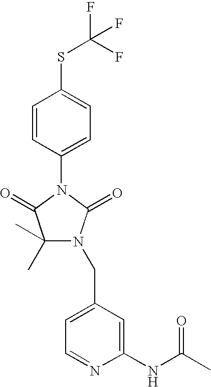

To a solution of 3 g of 1-[(2-chloropyridin-4-yl)methyl]-5,5-dimethyl-3-{4-[(trifluoromethyl)thio]phenyl}imidazolidine-2,4-dione obtained in stage d) of Example 1 in 60 mL of dioxane are successively added, under argon, 1.03 g of acetamide, 0.484 g of 9,9-dimethyl-4,5-bis(diphenylphosphino)xanthene (Xantphos), 0.156 g of palladium acetate and 7.96 g of caesium carbonate. The reaction mixture is heated at 90° C. for 5 hours and then filtered and concentrated under reduced pressure. The residue is purified by chromatography on a column of silica, eluting with a mixture of ethyl acetate and cyclohexane (50/50 by volume) to give 2.85 g of N-{4-[(5,5-dimethyl-2,4-dioxo-3-{4-[(trifluoromethyl)thio]phenyl}imidazolidin-1-yl)methyl]pyridin-2-yl}acetamide, the characteristics of which are as follows:

1H NMR spectrum at 400 MHz: 1.41 (s, 6H); 2.09 (s, 3H); 4.63 (s, 2H); 7.13 (dd, J=1.5 and 5.5 Hz, 1H); 7.66 (d, J=8.5 Hz, 2H); 7.88 (d, J=8.5 Hz, 2H); 8.11 (broad s, 1H); 8.24 (d, J=5.5 Hz, 1H); 10.5 (broad s, 1H).

Mass Spectrum (ES): m/z=453 [M+H]⁺
m/z=451 [M−H]⁻

### EXAMPLE 15

### 5,5-dimethyl-1-{[2-(pyrimidin-5-ylamino)pyrimidin-4-yl]methyl}-3-{4-[(trifluoromethyl)thio]phenyl}imidazolidine-2,4-dione

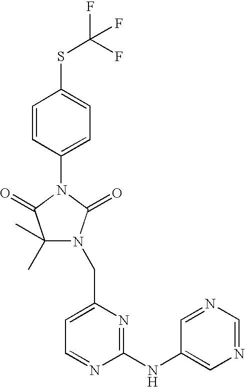

To a solution of 0.13 g of 1-[(2-aminopyrimidin-4-yl)methyl]-5,5-dimethyl-3-{4-[(trifluoromethyl)thio]phenyl}imidazolidine-2,4-dione obtained in stage g) of Example 10 in 10 mL of dioxane are successively added, under argon, 0.075 g of 5-bromopyrimidine, 0.02 g of 9,9-dimethyl-4,5-bis(diphenyl-phosphino)xanthene (Xantphos), 0.007 g of palladium acetate and 0.39 g of caesium carbonate. The reaction mixture is refluxed for 15 hours and then filtered and concentrated under reduced pressure. The residue is purified by chromatography on a column of silica, eluting with a mixture of dichloromethane and methanol (98/2 by volume) to give 0.043 g of 5,5-dimethyl-1-{[2-(pyrimidin-5-ylamino)pyrimidin-4-yl]methyl}-3-{4-[(tri-fluoromethyl)thio]phenyl}imidazolidine-2,4-dione, the characteristics of which are as follows:

1H NMR spectrum at 400 MHz: 1.45 (s, 6H); 4.64 (s, 2H); 7.06 (d, J=5.5 Hz, 1H); 7.71 (d, J=8.5 Hz, 2H); 7.86 (d, J=8.5 Hz, 2H); 8.52 (d, J=5.5 Hz, 1H); 8.76 (s, 1H); 9.18 (s, 2H); 10.0 (s, 1H).

Mass Spectrum (ES): m/z=490 [M+H]⁺
m/z=488 [M−H]⁻

### EXAMPLE 16

### 5,5-dimethyl-1-{[2-(pyrimidin-5-ylamino)pyrimidin-4-yl]methyl}-3-{4-[(trifluoromethyl)sulfonyl]phenyl}imidazolidine-2,4-dione

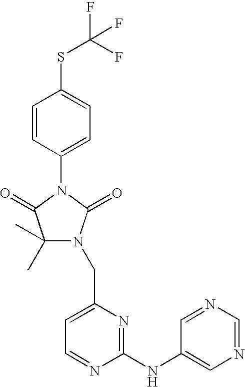

To a solution of 0.36 g of 1-[(2-aminopyrimidin-4-yl)methyl]-5,5-dimethyl-3-{4-[(trifluoromethyl)thio]phenyl}imidazolidine-2,4-dione obtained in stage g) of Example 10 in 20 mL of dioxane are successively added, under argon, 0.19 g of 5-bromopyrimidine, 0.055 g of 9,9-dimethyl-4,5-bis(diphenyl-phosphino)xanthene (Xantphos), 0.018 g of palladium acetate and 1 g of caesium carbonate. The reaction mixture is refluxed for 15 hours and then filtered and concentrated under reduced pressure. The residue is purified by chromatography on a column of silica, eluting with a mixture of dichloromethane and methanol (98/2 by volume) to give 0.16 g of 5,5-dimethyl-1-{[2-(pyrimidin-5-ylamino)pyrimidin-4-yl]methyl}-3-{4-[(trifluoro-methyl)sulfonyl]phenyl}imidazolidine-2,4-dione, the characteristics of which are as follows:

1H NMR spectrum at 400 MHz: 1.48 (s, 6H); 4.67 (s, 2H); 7.10 (d, J=5.5 Hz, 1H); 8.08 (d, J=8.5 Hz, 2H); 8.30 (d, J=8.5 Hz, 2H); 8.52 (d, J=5.5 Hz, 1H); 8.76 (s, 1H); 9.19 (s, 2H) 10.0 (s, 1H).

Mass Spectrum (ES): m/z=522 [M+H]⁺
m/z=520 [M−H]⁻

### EXAMPLE 17

### 1-({2-[(5-fluoropyridin-3-yl)amino]pyridin-4-yl}-methyl)-5,5-dimethyl-3-{4-[(trifluoromethyl)thio]phenyl}imidazolidine-2,4-dione

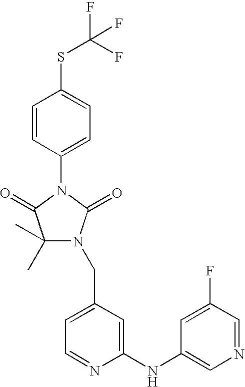

To a solution of 0.2 g of 1-[(2-aminopyridin-4-yl)methyl]-5,5-dimethyl-3-{4-[(trifluoromethyl)thio]phenyl}imidazolidine-2,4-dione obtained in stage b) of Example 14 in 5 mL of dioxane are successively added, under argon, 0.087 g of 5-bromo-3-fluoropyridine, 0.025 g of 9,9-dimethyl-4,5-bis(diphenyl-phosphino)xanthene (Xantphos), 0.010 g of palladium acetate and 0.7 g of caesium carbonate. The reaction mixture is refluxed for 3.5 hours and then filtered and concentrated under reduced pressure. The residue is purified by chromatography on a column of silica, eluting with a gradient of acetone in dichloro-methane to give 0.17 g of 1-({2-[(5-fluoropyridin-3-yl)amino]-pyridin-4-yl}methyl)-5,5-dimethyl-3-{4-[(trifluoromethyl)thio]-phenyl}imidazolidine-2,4-dione, the characteristics of which are as follows:

1H NMR spectrum at 400 MHz: 1.45 (s, 6H); 4.59 (s, 2H); 6.90 (broad s, 1H); 6.92 (broad d, J=5.5 Hz, 1H); 7.69 (d, J=8.5 Hz, 2H); 7.88 (d, J=8.5 Hz, 2H); 8.05 (d, J=2.5 Hz, 1H); 8.20 (d, J=5.5 Hz, 1H); 8.38 (td, J=2.5 and 12.5 Hz, 1H); 8.50 (t, J=2.5 Hz, 1H); 9.55 (broad m, 1H).

Mass Spectrum (ES): m/z=506 [M+H]⁺

The present invention especially includes the products of formula (I) belonging to formula (Ia) below:

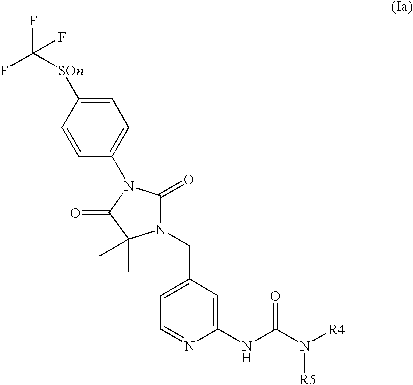

in which n and NR4R5 have the meanings given above.

The products of formula (Ia) may especially be prepared as indicated in the General Scheme 3 in two stages (compounds Z and AA).

The products of formula (Ia) as defined above in which the radical NR4R5 has the values given above numbered as ex 18 to ex 40 correspond, respectively, to Examples 18 to 40 belonging to the present invention: the preparation of the product of Example 18 is described below and the products of Examples 19 to 43 are prepared as indicated for the products of Example 18, replacing in stage B) the 3-pyrrolidin-1-ylpropylamine with the appropriate corresponding intermediate of formula HNR4R5.

Examples of products bearing different radicals NR4R5 according to the present invention are given below:

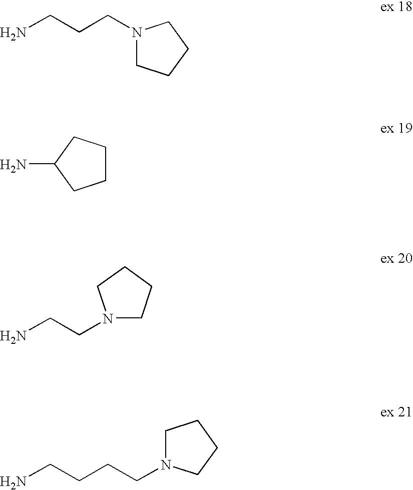

### EXAMPLE 18

### 1-{4-[(5,5-dimethyl-2,4-dioxo-3-{4-[(trifluoro-methyl)thio]phenyl}imidazolidin-1-yl)methyl]pyridin-2-yl}-3-(3-pyrrolidin-1-ylpropyl)urea

### Stage b)

### 1-{4-[(5,5-dimethyl-2,4-dioxo-3-{4-[(trifluoro-methyl)thio]phenyl}imidazolidin-1-yl)methyl]pyridin-2-yl}-3-(3-pyrrolidin-1-ylpropyl)urea

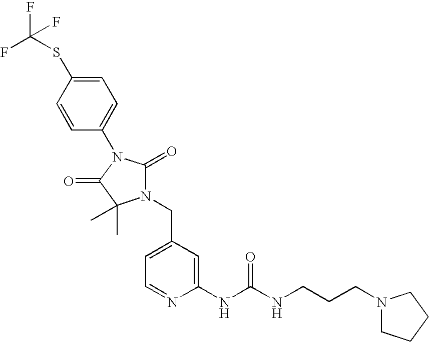

To a solution of 0.15 g of ethyl {{4-[(5,5-dimethyl-2,4-dioxo-3-{4-[(trifluoromethyl)thio]phenyl}imidazolidin-1-yl)methyl]-pyridin-2-yl}carbamate obtained in stage a) below in 2 mL of N-methylpyrrolidinone are added 0.316 mL of 3-pyrrolidin-1-yl-propylamine. The solution is heated at 130° C. by microwave for 1 hour. The reaction mixture is then diluted with 10 mL of distilled water and extracted with 3 times 30 mL of ethyl acetate. The combined organic phases are concentrated under reduced pressure and the residue is purified by chromatography on a column of silica (eluting with a gradient of dichloro-methane and a mixture of methanol and aqueous ammonia at 85/15 by volume) to give 0.072 g of 1-{4-[(5,5-dimethyl-2,4-dioxo-3-{4-[(trifluoromethyl)thio]phenyl}imidazolidin-1-yl)methyl]pyridin-2-yl}-3-(3-pyrrolidin-1-ylpropyl)urea, the characteristics of which are as follows:

1H NMR spectrum at 400 MHz: 1.42 (s, 6H); 1.61 (m, 2H); 1.67 (m, 4H); 2.41 (m, 6H); 3.20 (q, J=6.5 Hz, 2H); 4.56 (s, 2H); 6.94 (broad d, J=5.5 Hz, 1H); 7.32 (broad s, 1H); 7.67 (d, J=9.0 Hz, 2H); 7.87 (d, J=9.0 Hz, 2H); 8.11 (d, J=5.5 Hz, 1H); 8.27 (m, 1H); 9.11 (s, 1H).

Mass Spectrum (ES): m/z=565, [M+H]⁺ base peak

### Stage a)

### ethyl {4-[(5,5-dimethyl-2,4-dioxo-3-{4-[(trifluoromethyl)thio]phenyl}imidazolidin-1-yl)methyl]pyridin-2-yl}carbamate

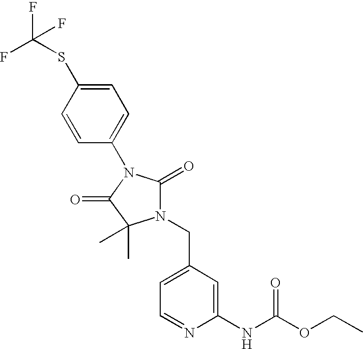

To a solution of 4.3 g of 1-[(2-chloropyridin-4-yl)methyl]-5,5-dimethyl-3-{4-[(trifluoromethyl)thio]phenyl}imidazolidine-2,4-dione obtained in stage d) of Example 1 in 105 mL of dioxane are successively added, under argon, 1.36 g of ethyl carbamate, 12.38 g caesium carbonate, 0.22 g of palladium acetate and 0.58 g of 9,9-dimethyl-4,5-bis(diphenylphosphino)xanthene. The reaction mixture is refluxed for 2 hours, filtered and concentrated under reduced pressure. The residue is triturated from diethyl ether to give 3.56 g of ethyl {{4-[(5,5-dimethyl-2,4-dioxo-3-{4-[(trifluoromethyl)thio]phenyl}imidazolidin-1-yl)methyl]pyridin-2-yl}carbamate, the characteristics of which are as follows:

1H NMR spectrum at 400 MHz: 1.23 (t, J=7.5 Hz, 3H); 1.42 (s, 6H); 4.14 (q, J=7.5 Hz, 2H); 4.62 (s, 2H); 7.09 (dd, J=1.5 and 5.5 Hz, 1H); 7.66 (d, J=8.5 Hz, 2H); 7.86 (m, 3H); 8.20 (d, J=5.5 Hz, 1H); 10.1 (broad s, 1H).

Mass Spectrum (1E): m/z=482 M+. base peak
m/z=467 (M —CH3)+
m/z=410 (M —CO2C2H5)+.

### EXAMPLE 19

### 1-cyclopentyl-3-{4-[(5,5-dimethyl-2,4-dioxo-3-{4-[(trifluoromethyl)thio]phenyl}imidazolidin-1-yl)methyl]pyridin-2-yl}urea

This product was prepared as in stage b) of Example 18, replacing the 3-pyrrolidin-1-ylpropylamine with cyclopentylamine and the N-methylpyrrolidinone with tetrahydrofuran, with heating for 2 hours at 140° C., to give 97 mg of 1-cyclopentyl-3-{4-[(5,5-dimethyl-2,4-dioxo-3-{4-[(trifluoromethyl)thio]phenyl}imidazolidin-1-yl)methyl]pyridin-2-yl}urea, the characteristics of which are as follows:

1H NMR spectrum at 400 MHz: 1.39 (partially masked m, 2H); 1.42 (s, 6H); from 1.50 to 1.72 (m, 4H); 1.86 (m, 2H); 4.00 (m, 1H); 4.58 (s, 2H); 6.94 (broad d, J=5.5 Hz, 1H); 7.36 (broad s, 1H); 7.65 (d, J=8.5 Hz, 2H); 7.87 (d, J=8.5 Hz, 2H); 8.11 (d, J=5.5 Hz, 1H); 8.20 (broad d, J=7.5 Hz, 1H); 9.02 (s, 1H).

Mass Spectrum (ES): m/Z=522 [M+H]⁺
m/Z==520; [M−H]−

### EXAMPLE 20

### 1-{4-[(5,5-dimethyl-2,4-dioxo-3-{4-[(trifluoro-methyl)thio]phenyl}imidazolidin-1-yl)methyl]pyridin-2-yl}-3-(2-pyrrolidin-1-ylethyl)urea

This product was prepared as in stage b) of Example 18, replacing the 3-pyrrolidin-1-ylpropylamine with N-(2-aminoethyl)pyrrolidine to give 93 mg of 1-{4-[(5,5-dimethyl-2,4-dioxo-3-{4-[(trifluoromethyl)thio]phenyl}imidazolidin-1-yl)methyl]pyridin-2-yl}-3-(2-pyrrolidin-1-ylethyl)urea, the characteristics of which are as follows:

1H NMR spectrum at 400 MHz: 1.42 (s, 6H); 1.69 (m, 4H); 2.47 (m, 4H); 2.52 (partially masked t, J=6.5 Hz, 2H); 3.27 (q, J=6.5 Hz, 2H); 4.58 (s, 2H); 6.94 (broad d, J=5.5 Hz, 1H); 7.36 (broad s, 1H); 7.67 (d, J=9.0 Hz, 2H); 7.87 (d, J=9.0 Hz, 2H); 8.10 (d, J=5.5 Hz, 1H); 8.25 (broad m, 1H); 9.17 (s, 1H).

Mass Spectrum (ES): m/z=551 [M+H]⁺
m/z=411; [MH—C7H12N2O]+
m/z=141; C7H13N2O+ base peak

### EXAMPLE 21

### 1-{4-[(5,5-dimethyl-2,4-dioxo-3-{4-[(trifluoro-methyl)thio]phenyl}imidazolidin-1-yl)methyl]pyridin-2-yl}-3-(4-pyrrolidin-1-ylbutyl)urea

This product was prepared as in stage b) of Example 18, replacing the 3-pyrrolidin-1-ylpropylamine with 1-(4-aminobutyl)pyrrolidine to give 100 mg of 1-{4-[(5,5-dimethyl-2,4-dioxo-3-{4-[(trifluoromethyl)thio]phenyl}imidazolidin-1-yl)methyl]pyridin-2-yl}-3-(4-pyrrolidin-1-ylbutyl)urea, the characteristics of which are as follows:

1H NMR spectrum at 400 MHz: 1.42 (s, 6H); 1.47 (m, 4H); 1.65 (m, 4H); 2.38 (m, 6H); 3.17 (q, J=6.5 Hz, 2H); 4.58 (s, 2H); 6.95 (broad d, J=5.5 Hz, 1H); 7.32 (broad s, 1H); 7.67 (d, J=9.0 Hz, 2H); 7.87 (d, J=9.0 Hz, 2H); 8.12 (d, J=5.5 Hz, 1H); 8.26 (broad m, 1H); 9.12 (s, 1H).

Mass Spectrum (ES): m/z=579 [M+H]⁺
m/z=290; [M+2H]2+/2 base peak

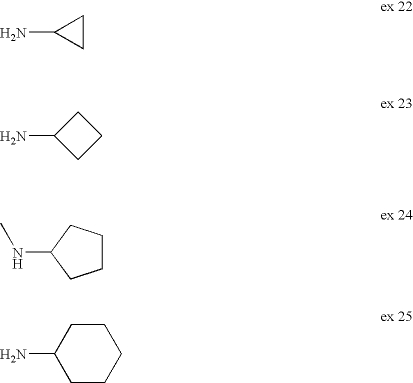

### EXAMPLE 22

### 1-cyclopropyl-3-{4-[(5,5-dimethyl-2,4-dioxo-3-{4-[(trifluoromethyl)thio]phenyl}imidazolidin-1-yl)methyl]pyridin-2-yl}urea

This product was prepared as in stage b) of Example 18, replacing the 3-pyrrolidin-1-ylpropylamine with cyclopropylamine and the N-methylpyrrolidinone with tetrahydrofuran, with heating for 2 hours at 140° C., to give 110 mg of 1-cyclopropyl-3-{4-[(5,5-dimethyl-2,4-dioxo-3-{4-[(trifluoromethyl)thio]phenyl}imidazolidin-1-yl)methyl]pyridin-2-yl}urea, the characteristics of which are as follows:

1H NMR spectrum at 400 MHz: 0.44 (m, 2H); 0.66 (m, 2H); 1.42 (s, 6H); 2.60 (m, 1H); 4.56 (s, 2H); 6.95 (dd, J=1.5 Hz, 1H); 7.37 (broad s, 1H); 7.67 (d, J=8.5 Hz, 2H); 7.87 (d, J=8.5 Hz, 2H); 8.11 (d, J=5.5 Hz, 1H); 8.23 (broad m, 1H); 9.06 (s, 1H).

Mass Spectrum (ES): m/z=494 [M+H]⁺
m/z=492; [M−H]−
m/z=538; MH-+HCO2H
m/z=409 [M+H]^{+-C}4H6NO

### EXAMPLE 23

### 1-cyclobutyl-3-{4-[(5,5-dimethyl-2,4-dioxo-3-{4-[(trifluoromethyl)thio]phenyl}imidazolidin-1-yl)methyl]pyridin-2-yl}urea

This product was prepared as in stage b) of Example 18, replacing the 3-pyrrolidin-1-ylpropylamine with cyclobutylamine and the N-methylpyrrolidinone with methanol, to give 50 mg of 1-cyclobutyl-3-{4-[(5,5-dimethyl-2,4-dioxo-3-{4-[(trifluoro-methyl)thio]phenyl}imidazolidin-1-yl)methyl]pyridin-2-yl}urea, the characteristics of which are as follows:

1H NMR spectrum at 400 MHz: 1.42 (s, 6H); from 1.55 to 1.70 (m, 2H); from 1.81 to 1.94 (m, 2H); from 2.18 to 2.27 (m, 2H); 4.18 (m, 1H); 4.58 (s, 2H); 6.96 (dd, J=1.5 and 5.5 Hz, 1H); 7.37 (broad s, 1H); 7.66 (d, J=9.0 Hz, 2H); 7.87 (d, J=9.0 Hz, 2H); 8.13 (d, J=5.5 Hz, 1H); 8.36 (broad d, J=7.5 Hz, 1H); 9.06 (s, 1H).

Mass Spectrum (ES): m/z=508 [M+H]⁺
m/z=506; [M−H]−

### EXAMPLE 24

### 1-cyclopentyl-3-{4-[(5,5-dimethyl-2,4-dioxo-3-{4-[(trifluoromethyl)thio]phenyl}imidazolidin-1-yl)methyl]pyridin-2-yl}-1-methylurea

This product was prepared as in stage b) of Example 18, replacing the 3-pyrrolidin-1-ylpropylamine with N-methylcyclopentylamine and the N-methylpyrrolidinone with tetrahydrofuran, to give 56 mg of 1-cyclopentyl-3-{4-[(5,5-dimethyl-2,4-dioxo-3-{4-[(trifluoromethyl)thio]phenyl}imidazolidin-1-yl)methyl]pyridin-2-yl}-1-methylurea, the characteristics of which are as follows:

1H NMR spectrum at 400 MHz: 1.41 (s, 6H); from 1.45 to 1.80 (m, 8H); 2.81 (s, 3H); 4.60 (s, 2H); 4.61 (m, 1H); 7.01 (broad d, J=5.5 Hz, 1H); 7.68 (d, J=8.5 Hz, 2H); 7.86 (m, 3H); 8.18 (d, J=5.5 Hz, 1H); 8.73 (s, 1H).

Mass Spectrum (ES): m/z=536 [M+H]⁺
m/z=534 [M−H]⁻

### EXAMPLE 25

### 1-cyclohexyl-3-{4-[(5,5-dimethyl-2,4-dioxo-3-{4-[(trifluoromethyl)thio]phenyl}imidazolidin-1-yl)methyl]pyridin-2-yl}urea

This product was prepared as in stage b) of Example 18, replacing the 3-pyrrolidin-1-ylpropylamine with cyclohexylamine and the N-methylpyrrolidinone with tetrahydrofuran, to give 90 mg of 1-cyclohexyl-3-{4-[(5,5-dimethyl-2,4-dioxo-3-{4-[(tri-fluoromethyl)thio]phenyl}imidazolidin-1-yl)methyl]pyridin-2-yl}urea, the characteristics of which are as follows:

1H NMR spectrum at 400 MHz: from 1.16 to 1.39 (m, 5H); 1.42 (s, 6H); 1.53 (m, 1H); 1.65 (m, 2H); 1.82 (m, 2H); 3.56 (m, 1H); 4.58 (s, 2H); 6.94 (broad d, J=5.5 Hz, 1H); 7.33 (broad s, 1H); 7.67 (d, J=8.5 Hz, 2H); 7.87 (d, J=8.5 Hz, 2H); 8.12 (d, J=5.5 Hz, 1H); 8.23 (broad d, J=7.5 Hz, 1H); 9.06 (s, 1H).

Mass Spectrum (ES): m/z=536 [M+H]⁺
m/z=534; [M−H]−
MH−+HCO2H=580−

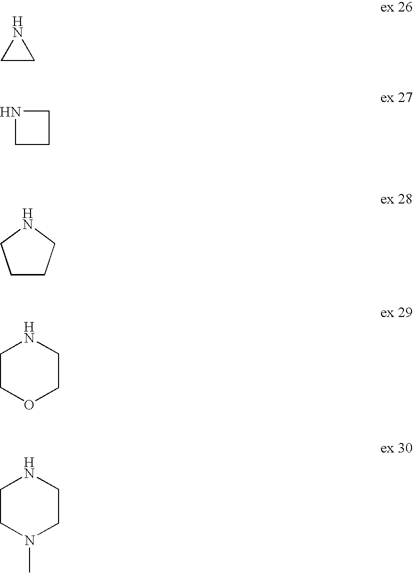

### EXAMPLE 26

### N-{4-[(5,5-dimethyl-2,4-dioxo-3-{4-[(trifluoro-methyl)thio]phenyl}imidazolidin-1-yl)methyl]pyridin-2-yl}aziridine-1-carboxamide

### EXAMPLE 27

### N-{4-[(5,5-dimethyl-2,4-dioxo-3-{4-[(trifluoro-methyl)thio]phenyl}imidazolidin-1-yl)methyl]pyridin-2-yl}azetidine-1-carboxamide

This product was prepared as in stage b) of Example 18, replacing the 3-pyrrolidin-1-ylpropylamine with azetidine and the N-methylpyrrolidinone with tetrahydrofuran, to give 65 mg of N-{4-[(5,5-dimethyl-2,4-dioxo-3-{4-[(trifluoromethyl)thio]-phenyl}imidazolidin-1-yl)methyl]pyridin-2-yl}azetidine-1-carboxamide, the characteristics of which are as follows:

1H NMR spectrum at 400 MHz: 1.42 (s, 6H); 2.15 (m, 2H); 3.98 (t, J=7.5 Hz, 4H); 4.59 (s, 2H); 7.01 (broad d, J=5.5 Hz, 1H); 7.66 (d, J=8.5 Hz, 2H); 7.86 (d, J=8.5 Hz, 2H); 7.95 (broad s, 1H); 8.16 (d, J=5.5 Hz, 1H); 8.97 (s, 1H).

Mass Spectrum (ES): m/z=494 [M+H]⁺
[M−H]−=492− m/z=492; [M−H]

### EXAMPLE 28

### N-{4-[(5,5-dimethyl-2,4-dioxo-3-{4-[(trifluoromethyl)thio]phenyl}imidazolidin-1-yl)methyl]pyridin-2-yl}pyrrolidine-1-carboxamide

This product was prepared as in stage b) of Example 18, replacing the 3-pyrrolidin-1-ylpropylamine with pyrrolidine to give 40 mg of N-{4-[(5,5-dimethyl-2,4-dioxo-3-{4-[(trifluoro-methyl)thio]phenyl}imidazolidin-1-yl)methyl]pyridin-2-yl}pyrrolidine-1-carboxamide, the characteristics of which are as follows:

1H NMR spectrum at 400 MHz: 1.42 (s, 6H); 1.83 (m, 4H); 3.39 (m, 4H); 4.59 (s, 2H); 7.01 (dd, J=1.5 and 5.0 Hz, 1H); 7.67 (d, J=9.0 Hz, 2H); 7.86 (d, J=9.0 Hz, 2H); 7.96 (broad s, 1H); 8.16 (d, J=5.0 Hz, 1H); 8.60 (s, 1H).

Mass Spectrum (ES): m/z=508 [M+H]⁺

### EXAMPLE 29

### N-{4-[(5,5-dimethyl-2,4-dioxo-3-{4-[(trifluoro-methyl)thio]phenyl}imidazolidin-1-yl)methyl]pyridin-2-yl}morpholine-4-carboxamide

This product was prepared as in stage b) of Example 18, replacing the 3-pyrrolidin-1-ylpropylamine with morpholine and the N-methylpyrrolidinone with tetrahydrofuran, to give 84 mg of N-{4-[(5,5-dimethyl-2,4-dioxo-3-{4-[(trifluoromethyl)thio]-phenyl}imidazolidin-1-yl)methyl]pyridin-2-yl}morpholine-4-carboxamide, the characteristics of which are as follows:

1H NMR spectrum at 400 MHz: 1.42 (s, 6H); 3.45 (m, 4H); 3.58 (m, 4H); 4.60 (s, 2H); 7.03 (broad d, J=5.5 Hz, 1H); 7.67 (d, J=8.5 Hz, 2H); 7.84 (broad s, 1H); 7.86 (d, J=8.5 Hz, 2H); 8.18 (d, J=5.5 Hz, 1H); 9.19 (s, 1H)

Mass Spectrum (ES): m/z=524 [M+H]⁺
m/z=522; [M−H]−

### EXAMPLE 30

### N-{4-[(5,5-dimethyl-2,4-dioxo-3-{4-[(trifluoro-methyl)thio]phenyl}imidazolidin-1-yl)methyl]pyridin-2-yl}-4-methylpiperazine-1-carboxamide

This product was prepared as in stage b) of Example 18, replacing the 3-pyrrolidin-1-ylpropylamine with N-methylpiperazine and the N-methylpyrrolidinone with tetrahydrofuran, to give 50 mg of N-{4-[(5,5-dimethyl-2,4-dioxo-3-{4-[(trifluoromethyl)thio]phenyl}imidazolidin-1-yl)-methyl]pyridin-2-yl}-4-methylpiperazine-1-carboxamide, the characteristics of which are as follows:

1H NMR spectrum at 400 MHz: 1.41 (s, 6H); 2.18 (s, 3H); 2.29 (m, 4H); 3.45 (m, 4H); 4.59 (s, 2H); 7.02 (broad d, J=5.5 Hz, 1H); 7.66 (d, J=8.5 Hz, 2H); 7.82 (broad s, 1H); 7.86 (d, J=8.5 Hz, 2H); 8.17 (d, J=5.5 Hz, 1H); 9.12 (s, 1H)

Mass Spectrum (ES): m/z=537 [M+H]⁺
m/z=535; [M−H]−
m/z=437 [M+H]⁺—C5H11N2

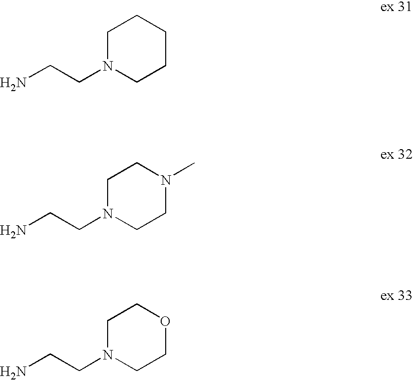

### EXAMPLE 31

### 1-{4-[(5,5-dimethyl-2,4-dioxo-3-{4-[(trifluoro-methyl)thio]phenyl}imidazolidin-1-yl)methyl]pyridin-2-yl}-3-(2-piperidin-1-ylethyl)urea

This product was prepared as in stage b) of Example 18, replacing the 3-pyrrolidin-1-ylpropylamine with 1-(2-aminoethyl)piperidine and the N-methylpyrrolidinone with tetrahydrofuran, to give 88 mg of 1-{4-[(5,5-dimethyl-2,4-dioxo-3-{4-[(trifluoromethyl)thio]phenyl}imidazolidin-1-yl)-methyl]pyridin-2-yl}-3-(2-piperidin-1-ylethyl)urea, the characteristics of which are as follows:

1H NMR spectrum at 400 MHz: from 1.35 to 1.55 (m, 6H); 1.42 (s, 6H); 2.36 (m, 6H); 3.26 (partially masked m, 2H); 4.58 (s, 2H); 6.94 (dd, J=1.5 and 5.5 Hz, 1H); 7.30 (broad s, 1H); 7.67 (d, J=8.5 Hz, 2H); 7.87 (d, J=8.5 Hz, 2H); 8.11 (d, J=5.5 Hz, 1H); 8.42 (broad m, 1H); 9.20 (s, 1H).

Mass Spectrum (ES): m/z=565 [M+H]⁺
m/z=563; [M−H]−

### EXAMPLE 32

### 1-{4-[(5,5-dimethyl-2,4-dioxo-3-{4-[(trifluoro-methyl)thio]phenyl}imidazolidin-1-yl)methyl]pyridin-2-yl}-3-[2-(4-methylpiperazin-1-yl)ethyl]urea

This product was prepared as in stage b) of Example 18, replacing the 3-pyrrolidin-1-ylpropylamine with 1-(2-aminoethyl)-4-methylpiperazine and the N-methylpyrrolidinone with tetrahydrofuran, to give 60 mg of 1-{4-[(5,5-dimethyl-2,4-dioxo-3-{4-[(trifluoromethyl)thio]phenyl}imidazolidin-1-yl)-methyl]pyridin-2-yl}-3-[2-(4-methylpiperazin-1-yl)ethyl]urea, the characteristics of which are as follows:

1H NMR spectrum at 400 MHz: 1.42 (s, 6H); from 2.35 to 2.42 (m, 6H); 3.27 (partially masked m, 2H); 3.59 (m, 4H); 4.58 (s, 2H); 6.95 (dd, J=1.5 and 5.5 Hz, 1H); 7.30 (broad s, 1H); 7.66 (d, J=8.5 Hz, 2H); 7.87 (d, J=8.5 Hz, 2H); 8.12 (d, J=5.5 Hz, 1H); 8.44 (broad m, 1H); 9.21 (s, 1H).

Mass Spectrum (ES): m/z=567 [M+H]⁺

### EXAMPLE 33

### 1-{4-[(5,5-dimethyl-2,4-dioxo-3-{4-[(trifluoro-methyl)thio]phenyl}imidazolidin-1-yl)methyl]pyridin-2-yl}-3-(2-morpholin-4-ylethyl)urea

This product was prepared as in stage b) of Example 18, replacing the 3-pyrrolidin-1-ylpropylamine with 1-(2-aminoethyl)morpholine and the N-methylpyrrolidinone with tetrahydrofuran, to give 110 mg of 1-{4-[(5,5-dimethyl-2,4-dioxo-3-{4-[(trifluoromethyl)thio]phenyl}imidazolidin-1-yl)-methyl]pyridin-2-yl}-3-(2-morpholin-4-ylethyl)urea, the characteristics of which are as follows:

1H NMR spectrum at 400 MHz: 1.42 (s, 6H); 2.15 (s, 3H); from 2.25 to 2.44 (m, 10H); 3.26 (partially masked m, 2H); 4.57 (s, 2H); 6.95 (broad d, J=5.5 Hz, 1H); 7.29 (broad s, 1H); 7.67 (d, J=8.5 Hz, 2H); 7.87 (d, J=8.5 Hz, 2H); 8.11 (d, J=5.5 Hz, 1H); 8.44 (broad m, 1H); 9.20 (s, 1H)

Mass Spectrum (ES): m/z=580 [M+H]⁺

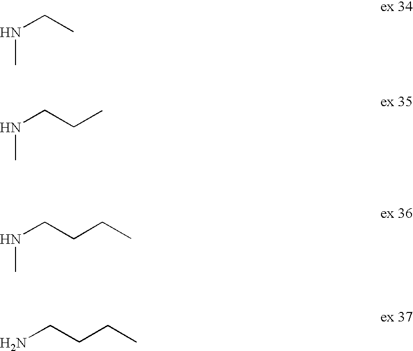

### EXAMPLE 34

### 3-{4-[(5,5-dimethyl-2,4-dioxo-3-{4-[(trifluoro-methyl)thio]phenyl}imidazolidin-1-yl)methyl]pyridin-2-yl}-1-ethyl-1-methylurea

This product was prepared as in stage b) of Example 18, replacing the 3-pyrrolidin-1-ylpropylamine with N-ethylmethylamine and the N-methylpyrrolidinone with tetrahydro-furan, to give 101 mg of 3-{4-[(5,5-dimethyl-2,4-dioxo-3-{4-[(trifluoromethyl)thio]phenyl}imidazolidin-1-yl)methyl]pyridin-2-yl}-1-ethyl-1-methylurea, the characteristics of which are as follows:

1H NMR spectrum at 400 MHz: 1.06 (t, J=7.0 Hz, 3H); 1.42 (s, 6H); 2.94 (s, 3H); 3.36 (d, J=7.0 Hz, 2H); 4.59 (s, 2H); 7.02 (dd, J=1.5 and 5.5 Hz, 1H); 7.67 (d, J=8.5 Hz, 2H); 7.85 (d, J=8.5 Hz, 2H); 7.87 (masked s, 1H); 8.17 (d, J=5.5 Hz, 1H); 8.72 (s, 1H).

Mass Spectrum (ES): m/z=496 [M+H]⁺
m/z=494; [M−H]−

### EXAMPLE 35

### 3-{4-[(5,5-dimethyl-2,4-dioxo-3-{4-[(trifluoromethyl)thio]phenyl}imidazolidin-1-yl)methyl]pyridin-2-yl}-1-methyl-1-propylurea

This product was prepared as in stage b) of Example 18, replacing the 3-pyrrolidin-1-ylpropylamine with N-methyl-N-propylamine and the N-methylpyrrolidinone with tetrahydrofuran, to give 100 mg of 3-{4-[(5,5-dimethyl-2,4-dioxo-3-{4-[(tri-fluoromethyl)thio]phenyl}imidazolidin-1-yl)methyl]pyridin-2-yl}-1-methyl-1-propylurea, the characteristics of which are as follows:

1H NMR spectrum at 400 MHz: 0.85 (t, J=7.0 Hz, 3H); 1.41 (s, 6H); 1.51 (m, 2H); 2.95 (s, 3H); 3.28 (masked m, 2H); 4.59 (s, 2H); 7.01 (dd, J=1.5 Hz and 5.5 Hz, 1H); 7.67 (d, J=8.5 Hz, 2H); 7.85 (d, J=8.5 Hz, 2H); 7.87 (masked s, 1H); 8.17 (d, J=5.5 Hz, 1H); 8.72 (s, 1H).

Mass Spectrum (ES): m/z=510 [M+H]⁺
m/z=510; [M−H]−

### EXAMPLE 36

### 1-butyl-3-{4-[(5,5-dimethyl-2,4-dioxo-3-{4-[(trifluoromethyl)thio]phenyl}imidazolidin-1-yl)methyl]pyridin-2-yl}-1-methylurea

This product was prepared as in stage b) of Example 18, replacing the 3-pyrrolidin-1-ylpropylamine with N-methyl-N-butylamine to give 40 mg of 1-butyl-3-{4-[(5,5-dimethyl-2,4-dioxo-3-{4-[(trifluoromethyl)thio]phenyl}imidazolidin-1-yl)-methyl]pyridin-2-yl}-1-methylurea, the characteristics of which are as follows:

1H NMR spectrum at 400 MHz: 0.89 (t, J=7.0 Hz, 3H); 1.27 (m, 2H); 1.42 (s, 6H); 1.47 (m, 2H); 2.94 (s, 3H); 3.30 (masked m, 2H); 4.59 (s, 2H); 7.01 (broad d, J=5.5 Hz, 1H); 7.69 (d, J=8.5 Hz, 2H); 7.85 (d, J=8.5 Hz, 2H); 7.86 (s, 1H); 8.17 (d, J=5.5 Hz, 1H); 8.72 (broad t, J=6.5 Hz, 1H).

Mass Spectrum (ES): m/z=524 [M+H]⁺
m/z=522; [M−H]−

### EXAMPLE 37

### 1-butyl-3-{4-[(5,5-dimethyl-2,4-dioxo-3-{4-[(tri-fluoromethyl)thio]phenyl}imidazolidin-1-yl)methyl]pyridin-2-yl}urea

This product was prepared as in stage b) of Example 18, replacing the 3-pyrrolidin-1-ylpropylamine with N-butylamine to give 40 mg of 1-butyl-3-{4-[(5,5-dimethyl-2,4-dioxo-3-{4-[(tri-fluoromethyl)thio]phenyl}imidazolidin-1-yl)methyl]pyridin-2-yl}urea, the characteristics of which are as follows:

1H NMR spectrum at 400 MHz: 0.90 (t, J=7.0 Hz, 3H); 1.31 (m, 2H); 1.42 (s, 6H); 1.44 (m, 2H); 3.17 (q, J=7.0 Hz, 2H); 4.58 (s, 2H); 6.94 (dd, J=1.5 and 5.5 Hz, 1H); 7.31 (broad s, 1H); 7.67 (d, J=8.5 Hz, 2H); 7.87 (d, J=8.5 Hz, 2H); 8.12 (d, J=5.5 Hz, 1H); 8.25 (broad t, J=7.0 Hz, 1H); 9.11 (s, 1H).

Mass Spectrum (ES): m/z=510 [M+H]⁺
m/z=508; [M−H]−

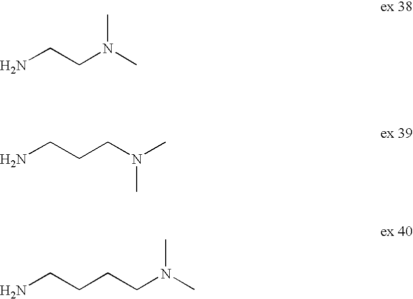

### EXAMPLE 38

### 1-[3-(dimethylamino)propyl]-3-{4-[(5,5-dimethyl-2,4-dioxo-3-{4-[(trifluoromethyl)thio]phenyl}imidazolidin-1-yl)methyl]pyridin-2-yl}urea

This product was prepared as in stage b) of Example 18, replacing the 3-pyrrolidin-1-ylpropylamine with N,N-dimethylethylenediamine and the N-methylpyrrolidinone with tetrahydrofuran, to give 54 mg of 1-[3-(dimethylamino)propyl]-3-{4-[(5,5-dimethyl-2,4-dioxo-3-{4-[(trifluoromethyl)thio]-phenyl}imidazolidin-1-yl)methyl]pyridin-2-yl}urea, the characteristics of which are as follows:

1H NMR spectrum at 400 MHz: 1.42 (s, 6H); 2.17 (s, 6H); 2.34 (t, J=6.5 Hz, 2H); 3.24 (q, J=6.5 Hz, 2H); 4.58 (s, 2H); 6.94 (broad d, J=5.5 Hz, 1H); 7.37 (broad s, 1H); 7.67 (d, J=8.5 Hz, 2H); 7.87 (d, J=8.5 Hz, 2H); 8.11 (d, J=5.5 Hz, 1H); 8.17 (broad t, J=6.5 Hz, 1H); 9.17 (s, 1H)

Mass Spectrum (ES): m/z=525 [M+H]⁺
m/z=523; [M−H]−

### EXAMPLE 39

### 1-[3-(dimethylamino)propyl]-3-{4-[(5,5-dimethyl-2,4-dioxo-3-{4-[(trifluoromethyl)thio]phenyl}imidazolidin-1-yl)methyl]pyridin-2-yl}urea

This product was prepared as in stage b) of Example 18, replacing the 3-pyrrolidin-1-ylpropylamine with N,N-dimethyl-1,3-propanediamine to give 106 mg of 1-[3-(dimethylamino)propyl]-3-{4-[(5,5-dimethyl-2,4-dioxo-3-{4-[(trifluoromethyl)thio]phenyl}imidazolidin-1-yl)methyl]pyridin-2-yl}urea, the characteristics of which are as follows:

1H NMR spectrum at 400 MHz: 1.42 (s, 6H); 1.59 (m, 2H); 2.12 (s, 6H); 2.23 (t, J=6.5 Hz, 2H); 3.18 (q, J=6.5 Hz, 2H); 4.58 (s, 2H); 6.95 (broad d, J=5.5 Hz, 1H); 7.31 (broad s, 1H); 7.67 (d, J=8.5 Hz, 2H); 7.87 (d, J=8.5 Hz, 2H); 8.11 (d, J=5.5 Hz, 1H); 8.31 (broad t, J=6.5 Hz, 1H); 9.13 (s, 1H).

Mass Spectrum (ES): m/z=539 [M+H]⁺
m/z=537 [M−H]⁻

### EXAMPLE 40

### 1-[4-(dimethylamino)butyl]-3-{4-[(5,5-dimethyl-2,4-dioxo-3-{4-[(trifluoromethyl)thio]phenyl}imidazolidin-1-yl)-methyl]pyridin-2-yl}urea

This product was prepared as in stage b) of Example 18, replacing the 3-pyrrolidin-1-ylpropylamine with N,N-dimethylaminobutylamine and the N-methylpyrrolidinone with tetrahydrofuran, to give 60 mg of 1-[4-(dimethylamino)butyl]-3-{4-[(5,5-dimethyl-2,4-dioxo-3-{4-[(trifluoromethyl)thio]-phenyl}imidazolidin-1-yl)methyl]pyridin-2-yl}urea, the characteristics of which are as follows:

1H NMR spectrum at 400 MHz: from 1.36 to 1.50 (m, 4H); 1.42 (s, 6H); 2.10 (s, 6H); 2.19 (t, J=6.5 Hz, 2H); 3.17 (q, J=6.5 Hz, 2H); 4.58 (s, 2H); 6.94 (broad d, J=5.5 Hz, 1H); 7.31 (broad s, 1H); 7.66 (d, J=8.5 Hz, 2H); 7.87 (d, J=8.5 Hz, 2H); 8.11 (d, J=5.5 Hz, 1H); 8.26 (broad t, J=6.5 Hz, 1H); 9.12 (s, 1H)

Mass Spectrum (ES): m/z=553 [M+H]⁺
m/z=551; [M−H]−

### EXAMPLE 40A

### 1-({2-[(5-fluoropyridin-3-yl)amino]pyridin-4-yl}-methyl)-5,5-dimethyl-3-{4-[(trifluoromethyl)thio]phenyl}imidazolidine-2,4-dione

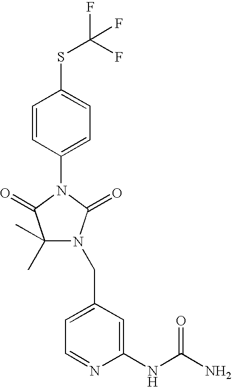

The product was prepared using the general method of Example 18 stage b) above, replacing the 3-pyrrolidin-1-ylpropylamine and the N-methylpyrrolidinone with a 7N solution of ammonia in methanol. The 1-({2-[(5-fluoropyridin-3-yl)amino]pyridin-4-yl}-methyl)-5,5-dimethyl-3-{4-[(trifluoromethyl)thio]phenyl}imidazolidine-2,4-dione is obtained in the form of a solid, the characteristics of which are as follows:

1H NMR spectrum at 400 MHz: 1.42 (s, 6H); 4.58 (s, 2H); 6.95 (broad d, J=5.5 Hz, 1H); 7.07 (very broad m, 2H); 7.38 (broad s, 1H); 7.66 (d, J=8.5 Hz, 2H); 7.87 (d, J=8.5 Hz, 2H); 8.12 (d, J=5.5 Hz, 1H); 9.08 (s, 1H)

Mass Spectrum (ES): m/z=454 [M+H]⁺
m/z=452; [M−H]−

### EXAMPLE 40B

### 1-{4-[(5,5-dimethyl-2,4-dioxo-3-{4-[(trifluoro-methyl)thio]phenyl}imidazolidin-1-yl)methyl]pyridin-2-yl}-3-[3-(pyrrolidin-1-ylmethyl)cyclobutyl]urea

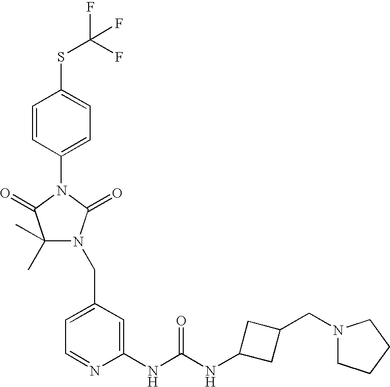

### Stage c)

### 1-{4-[(5,5-dimethyl-2,4-dioxo-3-{4-[(trifluoro-methyl)thio]phenyl}imidazolidin-1-yl)methyl]pyridin-2-yl}-3-[3-(pyrrolidin-1-ylmethyl)cyclobutyl]urea

To a solution of 22 mg of {3-[({4-[(5,5-dimethyl-2,4-dioxo-3-{4-[(trifluoromethyl)thio]phenyl}imidazolidin-1-yl)methyl]pyridin-2-yl}carbamoyl)amino]cyclobutyl}methyl methanesulfonate obtained in stage b) below in 0.8 mL of tetrahydrofuran are added 15 μL of pyrrolidine. The reaction mixture is heated by microwave at 130° C. for 1 hour and then concentrated under reduced pressure. The residue is purified by HPLC (water/acetonitrile gradient containing 0.1% formic acid) to give 7 mg of 1-{4-[(5,5-dimethyl-2,4-dioxo-3-{4-[(trifluoro-methyl)thio]phenyl}imidazolidin-1-yl)methyl]pyridin-2-yl}-3-[3-(pyrrolidin-1-ylmethyl)cyclobutyl]urea, the characteristics of which are as follows:

1H NMR spectrum at 400 MHz (60%/40% mixture of Cis and Trans isomers): 1.42 (s, 6H); 1.52 (m, 1H); 1.65 (m, 4H); from 1.95 to 2.57 (partially masked m, 10H); 4.03 (m, 0.6H); 4.22 (m, 0.4H); 4.57 (s, 2H); 6.96 (broad d, J=5.5 Hz, 1H); 7.37 (broad s, 1H); 7.67 (d, J=8.5 Hz, 2H); 7.87 (d, J=8.5 Hz, 2H); 8.13 (m, 1H); 8.24 (s, 1H); 8.27 (broad m, 0.6H); 8.38 (broad m, 0.4H); 9.02 (s, 0.6H); 9.04 (s, 0.4H).

Mass Spectrum: m/z=591 [M+H]⁺

### Stage b)

### {3-[({4-[(5,5-dimethyl-2,4-dioxo-3-{4-[(trifluoro-methyl)thio]phenyl}imidazolidin-1-yl)methyl]pyridin-2-yl}carbamoyl)amino]cyclobutyl}methyl methanesulfonate

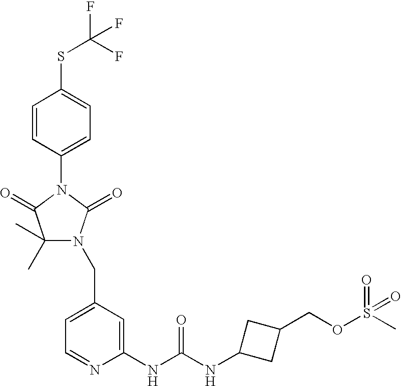

To a solution of 120 mg of 1-{4-[(5,5-dimethyl-2,4-dioxo-3-{4-[(trifluoromethyl)thio]phenyl}imidazolidin-1-yl)methyl]pyridin-2-yl}-3-[3-(hydroxymethyl)cyclobutyl]urea obtained in stage a) below in 10 mL of dichloromethane are added successively, under argon at 0° C., 2.7 mg of 4-N,N-dimethylaminopyridine, 46 μL of triethylamine and 26 μL of methanesulfonyl chloride. The reaction mixture is stirred for 1 hour at this temperature, the ice bath is then removed and 20 mL of saturated sodium hydrogen carbonate solution are added and the aqueous phase is extracted with twice 50 mL of ethyl acetate. The combined organic phases are washed with saturated sodium chloride solution, dried over magnesium sulfate, filtered and concentrated under reduced pressure. The residue is purified by chromatography on a column of silica, eluting with a mixture of ethyl acetate and dichloromethane (90/10 by volume) to give 95 mg of {3-[({4-[(5,5-dimethyl-2,4-dioxo-3-{4-[(trifluoromethyl)thio]phenyl}-imidazolidin-1-yl)methyl]pyridin-2-yl}carbamoyl)amino]-cyclobutyl}methyl methanesulfonate, the characteristics of which are as follows:

1H NMR spectrum at 400 MHz (60%/40% mixture of Cis and Trans isomers): 1.41 (s, 6H); from 1.65 to 2.43 (m, 5H); 3.18 (s, 1.8H); 3.20 (s, 1.2H); 4.12 (m, 0.6H); 4.20 (d, J=6.0 Hz, 0.4H); 4.29 (d, J=6.0 Hz, 0.4H); 4.32 (m, 0.4H); 4.59 (s, 2H); 6.98 (broad d, J=5.5 Hz, 1H); 7.35 (broad s, 0.4H); 7.38 (broad s, 0.6H); 7.68 (d, J=8.5 Hz, 2H); 7.89 (d, J=8.5 Hz, 2H); 8.14 (m, 1H); 8.32 (broad d, J=8.0 Hz, 0.6H); 8.48 (broad d, J=0.4H); 9.09 (s, 0.6H); 9.11 (s, 0.4H).

Mass Spectrum: m/z=616 [M+H]⁺
m/z=614 [M−H]⁻

### Stage a)

### 1-{4-[(5,5-dimethyl-2,4-dioxo-3-{4-[(trifluoro-methyl)thio]phenyl}imidazolidin-1-yl)methyl]pyridin-2-yl}-3-[3-(hydroxymethyl)cyclobutyl]urea

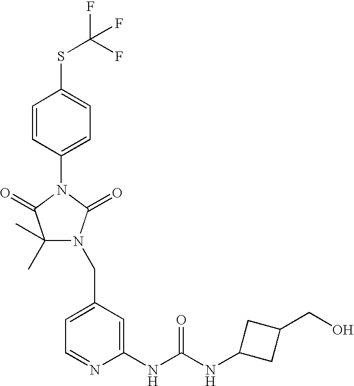

To a solution of 650 mg of ethyl {4-[(5,5-dimethyl-2,4-dioxo-3-{4-[(trifluoromethyl)thio]phenyl}imidazolidin-1-yl)methyl]pyridin-2-yl}carbamate obtained in stage a) of Example 18 in 3 mL of tetrahydrofuran are added 409 mg of (3-aminocyclobutyl)-methanol obtained according to the literature reference: Maruyama, T. et al. Chem. Pharm. Bull. (1990), 38(10), pp. 2719-2725. The reaction mixture is heated by microwave at 130° C. for 3 hours and then concentrated under reduced pressure. The residue is purified by HPLC (reverse-phase C18 column, eluting with a water/acetonitrile gradient containing 0.1% formic acid) to give 122 mg of 1-{4-[(5,5-dimethyl-2,4-dioxo-3-{4-[(trifluoromethyl)thio]phenyl}imidazolidin-1-yl)methyl]pyridin-2-yl}-3-[3-(hydroxymethyl)cyclobutyl]urea, the characteristics of which are as follows:

1H NMR spectrum at 400 MHz (60%/40% mixture of cis-trans isomers): 1.42 (s, 6H); 1.62 (m, 1H); from 1.85 to 2.32 (m, 4H); from 3.30 to 3.47 (partially masked m, 2H); 4.04 (m, 0.6H); 4.21 (m, 0.4H); 4.49 (t, J=5.5 Hz, 0.6H); 4.56 (t, J=5.5 Hz, 0.4H); 4.58 (s, 2H); 6.96 (broad d, J=5.5 Hz, 1H); 7.35 (broad s, 0.4H); 7.39 (broad s, 0.6H); 7.68 (d, J=8.5 Hz, 2H); 7.88 (d, J=8.5 Hz, 2H); 8.14 (m, 1H); 8.21 (broad d, J=8.0 Hz, 0.6H); 8.39 (broad d, J=8.0 Hz, 0.4H); 9.00 (s, 0.6H); 9.04 (s, 0.4H).

Mass Spectrum: m/z=538 [M+H]⁺
m/z=536 [M−H]⁻

### EXAMPLE 40C

### 1-({2-[(3-fluorophenyl)amino]pyridin-4-yl}methyl)-5,5-dimethyl-3-{4-[(trifluoromethyl)thio]phenyl}imidazolidine-2,4-dione

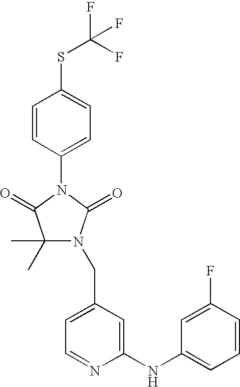

To a solution of 520 mg of 1-[(2-chloropyridin-4-yl)methyl]-5,5-dimethyl-3-{4-[(trifluoromethyl)thio]phenyl}imidazolidine-2,4-dione obtained in stage d) of Example 1 in 15 mL of dioxane are successively added, under argon, 27 mg of palladium diacetate, 84 mg of (9,9-dimethyl-9H-xanthene-3,6-diyl)bis(diphenylphosphine) (Xantphos), 1.5 g of caesium carbonate and 269 mg of 3-fluoroaniline. The reaction mixture is heated at 100° C. for 1.5 hours and then filtered and the filtrate is concentrated under reduced pressure. The residue is purified by chromatography on a column of silica, eluting with a mixture of petroleum ether and ethyl acetate (70/30 by volume) to give 404 mg of 1-({2-[(3-fluorophenyl)amino]pyridin-4-yl}methyl)-5,5-dimethyl-3-{4-[(trifluoromethyl)thio]phenyl}-imidazolidine-2,4-dione, the characteristics of which are as follows:

1H NMR spectrum at 400 MHz: 1.44 (s, 6H); 4.57 (s, 2H); 6.66 (m, 1H); 6.85 (m, 2H); from 7.20 to 7.31 (m, 2H); 7.69 (d, J=8.5 Hz, 2H); 7.83 (td, J=1.5 and 12.0 Hz, 1H); 7.88 (d, J=8.5 Hz, 2H); 8.15 (d, J=5.5 Hz, 1H); 9.25 (s, 1H).

Mass Spectrum (ES): m/z=505 [M+H]⁺

### EXAMPLE 40D

### 1-{[2-(cyclopropylamino)pyridin-4-yl]methyl}-5,5-dimethyl-3-{4-[(trifluoromethyl)thio]phenyl}imidazolidine-2,4-dione

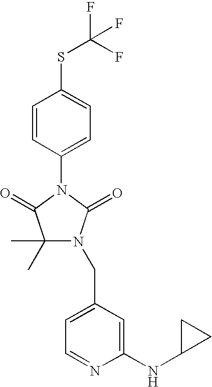

700 mg of 1-[(2-chloropyridin-4-yl)methyl]-5,5-dimethyl-3-{4-[(trifluoromethyl)thio]phenyl}imidazolidine-2,4-dione obtained in stage d) of Example 1 and 1.6 mL of cyclopropylamine are heated by microwave at 150° C. for 12 hours and then concentrated under reduced pressure. The residue is purified by chromatography on a column of silica, eluting with a mixture of petroleum ether and ethyl acetate (50/50 by volume) to give 65 mg of 1-{[2-(cyclopropylamino)pyridin-4-yl]methyl}-5,5-dimethyl-3-{4-[(trifluoromethyl)thio]phenyl}imidazolidine-2,4-dione, the characteristics of which are as follows:

1H NMR spectrum at 400 MHz: 0.40 (m, 2H); 0.68 (m, 2H); 1.41 (s, 6H); 2.50 (masked m, 1H); 4.49 (s, 2H); 6.58 (m, 2H); 6.70 (d, J=2.0 Hz, 1H); 7.65 (d, J=8.5 Hz, 2H); 7.87 (d, J=8.5 Hz, 2H); 7.92 (d, J=5.5 Hz, 1H).

Mass Spectrum (ES): m/z=451 [M+H]⁺

### EXAMPLE 40E

### 1-({2-[(2-chloropyridin-3-yl)amino]pyridin-4-yl}-methyl)-5,5-dimethyl-3-{4-[(trifluoromethyl)thio]phenyl}imidazolidine-2,4-dione

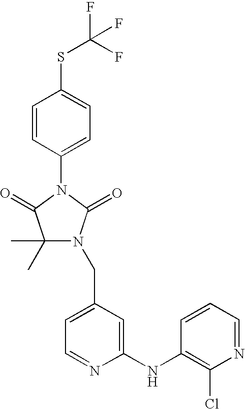

To a solution of 600 mg of 1-[(2-aminopyridin-4-yl)methyl]-5,5-dimethyl-3-{4-[(trifluoromethyl)thio]phenyl}imidazolidine-2,4-dione obtained in stage b) of Example 14 in 50 mL of dioxane are successively added, under argon, 33 mg of palladium diacetate, 100 mg of (9,9-dimethyl-9H-xanthene-3,6-diyl)bis(diphenylphosphine) (Xantphos), 1.81 g of caesium carbonate and 0.42 g of 2-chloro-3-iodopyridine. The reaction mixture is heated at 90° C. for 5 hours and then filtered and the filtrate is concentrated under reduced pressure. The residue is purified by chromatography on a column of silica, eluting with a mixture of cyclohexane and ethyl acetate (70/30 by volume) to give 0.47 g of 1-({2-[(2-chloropyridin-3-yl)amino]pyridin-4-yl}methyl)-5,5-dimethyl-3-{4-[(trifluoromethyl)thio]phenyl}-imidazolidine-2,4-dione, the characteristics of which are as follows:

1H NMR spectrum at 400 MHz: 1.46 (s, 6H); 4.58 (s, 2H); 6.90 (broad d, J=5.5 Hz, 1H); 7.12 (broad s, 1H); 7.35 (dd, J=5.5 and 8.5 Hz, 1H); 7.69 (d, J=8.5 Hz, 2H); 7.88 (d, J=8.5 Hz, 2H); 8.00 (dd, J=2.0 and 5.5 Hz, 1H); 8.10 (d, J=5.5 Hz, 1H); 8.47 (s, 1H); 8.56 (dd, J=2.0 and 8.5 Hz, 1H).

Mass Spectrum (ES): m/z=522 [M+H]⁺
m/z=520 [M−H]⁻

### EXAMPLE 40F

### 1-({2-[(6-chloropyridin-3-yl)amino]pyridin-4-yl}-methyl)-5,5-dimethyl-3-{4-[(trifluoromethyl)thio]phenyl}imidazolidine-2,4-dione

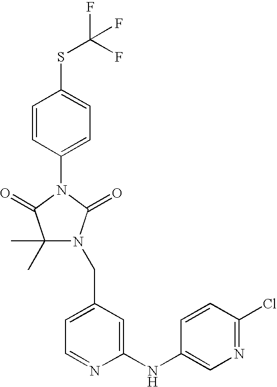

To a solution of 400 mg of 1-[(2-aminopyridin-4-yl)methyl]-5,5-dimethyl-3-{4-[(trifluoromethyl)thio]phenyl}imidazolidine-2,4-dione obtained in stage b) of Example 14 in 30 mL of dioxane are successively added, under argon, 22 mg of palladium diacetate, 67 mg of (9,9-dimethyl-9H-xanthene-3,6-diyl)bis(diphenylphosphine) (Xantphos), 1.2 g of caesium carbonate and 0.28 g of 2-chloro-5-iodopyridine. The reaction mixture is heated at 90° C. for 3 hours and then filtered and the filtrate is concentrated under reduced pressure. The residue is purified by chromatography on a column of silica, eluting with a mixture of cyclohexane and ethyl acetate (70/30 by volume) to give 0.38 g of 1-({2-[(6-chloropyridin-3-yl)amino]pyridin-4-yl}methyl)-5,5-dimethyl-3-{4-[(trifluoromethyl)thio]phenyl}-imidazolidine-2,4-dione, the characteristics of which are as follows:

1H NMR spectrum at 400 MHz: 1.43 (s, 6H); 4.59 (s, 2H); 6.86 (broad s, 1H); 6.89 (broad d, J=5.5 Hz, 1H); 7.38 (d, J=8.5 Hz, 1H); 7.69 (d, J=8.5 Hz, 2H); 7.88 (d, J=8.5 Hz, 2H); 8.14 (d, J=5.5 Hz, 1H); 8.27 (dd, J=2.5 and 8.5 Hz, 1H); 8.64 (d, J=2.5 Hz, 1H); 9.38 (s, 1H).

Mass Spectrum (ES): m/z=522 [M+H]⁺

### EXAMPLE 40G

### 1-({2-[(6-hydroxypyridin-3-yl)amino]pyridin-4-yl}-methyl)-5,5-dimethyl-3-{4-[(trifluoromethyl)thio]phenyl}imidazolidine-2,4-dione

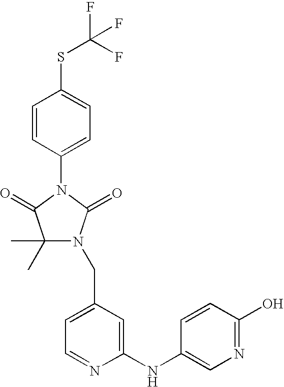

To a solution of 500 mg of 1-[(2-chloropyridin-4-yl)methyl]-5,5-dimethyl-3-{4-[(trifluoromethyl)thio]phenyl}imidazolidine-2,4-dione obtained in stage d) of Example 1 in 15 mL of dioxane are successively added, under argon, 52 mg of palladium diacetate, 160 mg of (9,9-dimethyl-9H-xanthene-3,6-diyl)bis(diphenylphosphine) (Xantphos), 1.74 g of caesium carbonate and 320 mg of 5-amino-2-hydroxypyridine. The reaction mixture is refluxed for 5 hours and then filtered and the filtrate is concentrated under reduced pressure. The residue is purified by chromatography on a column of silica, eluting with a mixture of dichloromethane and methanol (98/2 by volume) to give 11 mg of 1-({2-[(6-hydroxypyridin-3-yl)amino]pyridin-4-yl}methyl)-5,5-dimethyl-3-{4-[(trifluoromethyl)thio]phenyl}-imidazolidine-2,4-dione, the characteristics of which are as follows:

1H NMR spectrum at 400 MHz: 1.45 (s, 6H); 4.58 (s, 2H); 6.40 (d, J=10.0 Hz, 1H); 6.80 (m, 2H); 7.48 (broad d, J=10.0 Hz, 1H); 7.67 (d, J=8.5 Hz, 2H); 7.88 (m, 3H); 7.99 (d, J=5.0 Hz, 1H); 9.00 (broad m, 1H).

Mass Spectrum (ES): m/z=504 [M+H]⁺
m/z=502 [M−H]⁻

### EXAMPLE 40H

### 5,5-dimethyl-1-[(2-{[5-(pyrrolidin-1-yl-methyl)pyridin-3-yl]amino}pyridin-4-yl)methyl]-3-{4-[(tri-fluoromethyl)thio]phenyl}imidazolidine-2,4-dione

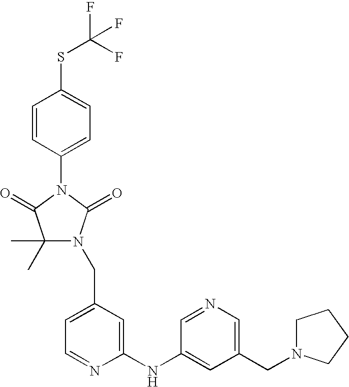

### Stage b)

### 5,5-dimethyl-1-[(2-{[5-(pyrrolidin-1-ylmethyl)pyri-din-3-yl]amino}pyridin-4-yl)methyl]-3-{4-[(trifluoromethyl)-thio]phenyl}imidazolidine-2,4-dione

To a solution of 360 mg of 1-[(2-aminopyridin-4-yl)methyl]-5,5-dimethyl-3-{4-[(trifluoromethyl)thio]phenyl}imidazolidine-2,4-dione obtained in stage b) of Example 14 in 25 mL of dioxane are successively added, under argon, 29 mg of palladium diacetate, 61 mg of (9,9-dimethyl-9H-xanthene-3,6-diyl)bis(diphenylphosphine) (Xantphos), 1.1 g of caesium carbonate and 0.25 g of 3-bromo-5-pyrrolidin-1-ylmethylpyridine obtained in stage a) below. The reaction mixture is refluxed for 5 hours and then filtered and the filtrate is concentrated under reduced pressure. The residue is purified by chromatography on a column of silica, eluting with a mixture of dichloromethane and methanol (96/4 by volume) to give 56 mg of 5,5-dimethyl-1-[(2-{[5-(pyrrolidin-1-ylmethyl)pyridin-3-yl]amino}pyridin-4-yl)methyl]-3-{4-[(trifluoromethyl)thio]-phenyl}imidazolidine-2,4-dione, the characteristics of which are as follows:

1H NMR spectrum at 400 MHz: 1.44 (s, 6H); 1.69 (m, 4H); 2.43 (m, 4H); 3.54 (s, 2H); 4.57 (s, 2H); 6.83 (m, 2H); 7.69 (d, J=8.5 Hz, 2H); 7.88 (d, J=8.5 Hz, 2H); 8.00 (d, J=2.5 Hz, 1H); 8.07 (t, J=2.5 Hz, 1H); 8.14 (d, J=5.5 Hz, 1H); 8.73 (d, J=2.5 Hz, 1H); 9.15 (s, 1H).

Mass Spectrum (ES): m/z=571 [M+H]⁺
m/z=569 [M−H]⁻

### Stage a)

### 3-Bromo-5-pyrrolidin-1-ylmethylpyridine

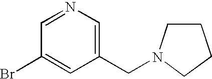

To a solution of 5-bromo-3-pyridinecarboxaldehyde in 20 mL of dichloro-1,2-ethane are successively added, under argon, 4.55 g of sodium triacetoxyborohydride and 0.94 mL of pyrrolidine. The reaction mixture is stirred at room temperature for 3 hours and then washed with saturated sodium hydrogen carbonate solution, with water and with saturated sodium chloride solution, dried over magnesium sulfate, filtered and concentrated under reduced pressure. The residue is purified by chromatography on a column of silica, eluting with a mixture of ethyl acetate and cyclohexane (80/20 by volume) to give 1.4 g of 3-bromo-5-pyrrolidin-1-ylmethylpyridine in the form of a pale yellow oil.

Mass Spectrum (ES): m/z=241 [M+H]⁺

m/z=161 [M+H]⁺−Br (base peak)

### EXAMPLE 40I

### 5,5-dimethyl-1-[(2-{[6-(pyrrolidin-1-yl-methyl)pyridin-3-yl]amino}pyridin-4-yl)methyl]-3-{4-[(trifluoromethyl)thio]phenyl}imidazolidine-2,4-dione

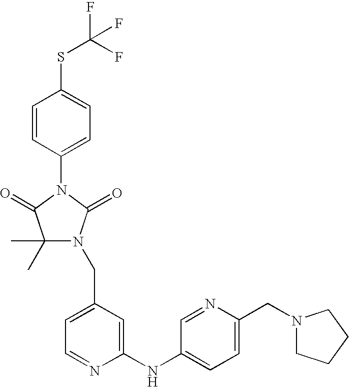

### Stage b)

### 5,5-dimethyl-1-[(2-{[6-(pyrrolidin-1-ylmethyl)pyridin-3-yl]amino}pyridin-4-yl)methyl]-3-{4-[(trifluoromethyl)-thio]phenyl}imidazolidine-2,4-dione

To a solution of 0.5 g of 1-[(2-aminopyridin-4-yl)methyl]-5,5-dimethyl-3-{4-[(trifluoromethyl)thio]phenyl}imidazolidine-2,4-dione obtained in stage b) of Example 14 in 15 mL of dioxane are successively added, under argon, 0.32 g of 5-bromo-2-pyrrolidin-1-ylmethylpyridine obtained in stage a) below, 77 mg of 9,9-dimethyl-4,5-bis(diphenylphosphino)xanthene (Xantphos), 38 mg of palladium acetate and 1.75 g of caesium carbonate. The reaction mixture is refluxed for 6 hours and then filtered and concentrated under reduced pressure. The residue is purified by chromatography on a column of silica, eluting with a mixture of dichloromethane and methanol (96/4 by volume) to give 0.1 g of 5,5-dimethyl-1-[(2-{[6-(pyrrolidin-1-ylmethyl)pyridin-3-yl]amino}pyridin-4-yl)methyl]-3-{4-[(trifluoromethyl)thio]-phenyl}imidazolidine-2,4-dione, the characteristics of which are as follows:

1H NMR spectrum at 400 MHz: 1.43 (s, 6H); 1.70 (m, 4H); 2.46 (m, 4H); 3.60 (s, 2H); 4.56 (s, 2H); 6.82 (m, 2H); 7.29 (d, J=8.5 Hz, 1H); 769 (d, J=8.5 Hz, 2H); 7.88 (d, J=8.5 Hz, 2H); 8.11 (d, J=5.5 Hz, 1H); 8.15 (dd, J=2.5 and 8.5 Hz, 1H); 8.64 (d, J=2.5 Hz, 1H); 9.12 (s, 1H).

Mass Spectrum (ES): m/z=571 [M+H]⁺
m/z=569 [M−H]⁻

### Stage a)

### 5-Bromo-2-pyrrolidin-1-ylmethylpyridine

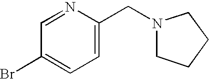

To a solution of 2 g of 5-bromo-2-formylpyridine in 20 mL of dichloro-1,2-ethane are successively added, under argon, 4.55 g of sodium triacetoxyborohydride and 0.94 mL of pyrrolidine. The reaction mixture is stirred at room temperature for 1 hour and then diluted with dichloromethane and the organic phase is washed with saturated sodium hydrogen carbonate solution, with water and with saturated sodium chloride solution, dried over magnesium sulfate, filtered and concentrated under reduced pressure. The residue is purified by chromatography on a column of silica, eluting with a mixture of dichloromethane and methanol (98/2 by volume) to give 0.93 g of 5-bromo-2-pyrrolidin-1-ylmethylpyridine, the characteristics of which are as follows:

1H NMR spectrum at 400 MHz: 1.70 (m, 4H); 2.48 (m, 4H); 3.69 (s, 2H); 7.40 (d, J=8.5 Hz, 1H); 7.99 (dd, J=2.5 and 8.5 Hz, 1H); 8.59 (d, J=2.5 Hz, 1H).

Mass Spectrum (ES): m/z=241 [M+H]⁺

### EXAMPLE 40J

### 1-[3-(azetidin-1-ylmethyl)cyclobutyl]-3-{4-[(5,5-dimethyl-2,4-dioxo-3-{4-[(trifluoromethyl)thio]phenyl}imidazolidin-1-yl)methyl]pyridin-2-yl}urea

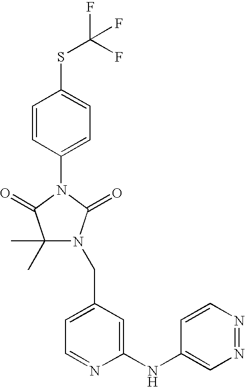

To a solution of 0.6 g of 1-[(2-chloropyridin-4-yl)methyl]-5,5-dimethyl-3-{4-[(trifluoromethyl)thio]phenyl}imidazolidine-2,4-dione obtained in stage d) of Example 1 in 15 mL of dioxane are successively added, under argon, 0.2 g of 4-aminopyridazine, 1.73 g of caesium carbonate, 97 mg of (9,9-dimethyl-9H-xanthene-3,6-diyl)bis(diphenylphosphine) (Xantphos) and 62 mg of palladium diacetate. The reaction mixture is refluxed for 5 hours, filtered and concentrated under reduced pressure. The residue is purified by chromatography on a column of silica, eluting with a mixture of dichloromethane and methanol (98/2 by volume) to give 50 mg of 1-[3-(azetidin-1-ylmethyl)cyclobutyl]-3-{4-[(5,5-dimethyl-2,4-dioxo-3-{4-[(trifluoromethyl)thio]-phenyl}imidazolidin-1-yl)methyl]pyridin-2-yl}urea, the characteristics of which are as follows:

1H NMR spectrum at 400 MHz: 1.43 (s, 6H); 4.61 (s, 2H); 6.99 (broad s, 1H); 7.03 (dd, J=1.5 and 5.5 Hz, 1H); 7.69 (d, J=8.5 Hz, 2H); 7.88 (d, J=8.5 Hz, 2H); 8.12 (dd, J=2.0 and 6.0 Hz, 1H); 8.28 (d, J=5.5 Hz, 1H); 8.82 (d, J=6.0 Hz, 1H); 9.26 (d, J=2.0 Hz, 1H); 9.80 (s, 1H).

Mass Spectrum (ES): m/z=489 [M+H]⁺
m/z=487 [M−H]⁻

### EXAMPLE 40K

### 1-[3-(azetidin-1-ylmethyl)cyclobutyl]-3-{4-[(5,5-dimethyl-2,4-dioxo-3-{4-[(trifluoromethyl)thio]phenyl}imidazolidin-1-yl)methyl]pyridin-2-yl}urea

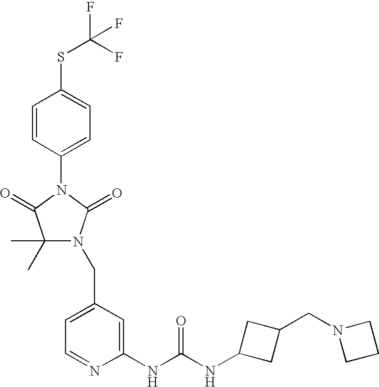

To a solution of 22 mg of {3-[({4-[(5,5-dimethyl-2,4-dioxo-3-{4-[(trifluoromethyl)thio]phenyl}imidazolidin-1-yl)methyl]pyridin-2-yl}carbamoyl)amino]cyclobutyl}methyl methanesulfonate obtained in stage b) of Example 40B in 0.8 mL of tetrahydrofuran are added 12 μL of azetidine. The reaction mixture is heated by microwave at 130° C. for 1 hour and then concentrated under reduced pressure. The residue is purified by HPLC (water/acetonitrile gradient containing 0.1% formic acid) to give 4 mg of 1-[3-(azetidin-1-ylmethyl)cyclobutyl]-3-{4-[(5,5-dimethyl-2,4-dioxo-3-{4-[(trifluoromethyl)thio]phenyl}-imidazolidin-1-yl)methyl]pyridin-2-yl}urea, the characteristics of which are as follows:

LCMS: TR=3.54 min; m/Z=577 [M+H]⁺; m/z=575 [M−H]⁻

### EXAMPLE 40L

### methyl {4-[(5,5-dimethyl-2,4-dioxo-3-{4-[(tri-fluoromethyl)thio]phenyl}imidazolidin-1-yl)methyl]pyridin-2-yl}carbamate

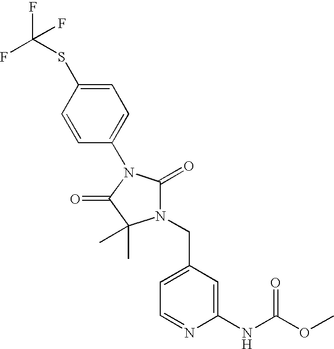

To a solution of 0.5 g of 1-[(2-chloropyridin-4-yl)methyl]-5,5-dimethyl-3-{4-[(trifluoromethyl)thio]phenyl}imidazolidine-2,4-dione obtained in stage d) of Example 1 in 15 mL of dioxane are successively added, under argon, 131 mg of methyl carbamate, 1.44 g of caesium carbonate, 26 mg of palladium acetate and 67 mg of 9,9-dimethyl-4,5-bis(diphenylphosphino)xanthene. The reaction mixture is refluxed for 1 hour, filtered and concentrated under reduced pressure. The residue is purified by chromatography on a column of silica, eluting with a gradient of dichloromethane and ethyl acetate (from 100/0 to 80/20 by volume) to give 243 mg of methyl {4-[(5,5-dimethyl-2,4-dioxo-3-{4-[(trifluoromethyl)thio]phenyl}imidazolidin-1-yl)methyl]pyri-din-2-yl}carbamate, the characteristics of which are as follows:

1H NMR spectrum at 400 MHz: 1.42 (s, 6H); 3.67 (s, 3H); 4.62 (s, 2H); 7.09 (dd, J=1.5 and 5.5 Hz, 1H); 7.66 (d, J=8.5 Hz, 2H); 7.86 (m, 3H); 8.20 (d, J=5.5 Hz, 1H); 10.1 (broad s, 1H).

Mass Spectrum (1E): m/z=467 M+. base peak

### In Vitro Biological Tests

### A) Experimental Protocol for the Kinase IGF-1R Test:

The inhibitory activity of the compounds on IGF1R is determined by measuring the inhibition of autophosphorylation of the enzyme using a time-resolved fluorescence test (HTRF). The human cytoplasmic domain of IGF-1R was cloned by fusion with glutathione S-transferase (GST) in the baculovirus expression vector pFastBac-GST. The protein is expressed in the SF21 cells and purified to about 80% homogeneity. For the enzymatic test, the test compound at 10 mM dissolved in DMSO is diluted in ⅓ steps in a 50 mM Hepes, pH 7.5, 5 mM MnCl₂, 50 mM NaCl, 3% Glycerol, 0.025% Tween 20 buffer. To measure the inhibition, the successive dilutions of the compound are preincubated for 30 minutes and 90 minutes in the presence of 5 nM of enzyme, the final DMSO concentration not exceeding 1%. The enzymatic reaction is initiated to have a final ATP concentration of 120 μM, and is stopped after 5 minutes by addition of 100 mM Hepes, pH 7.0 buffer containing 0.4 M of potassium fluoride, 133 mM EDTA, 0.1% BSA, the XL665-labelled antibody anti-GST and the anti-phosphotyrosine antibody conjugated to europium cryptate Eu—K (Cis-Bio Int.). The characteristics of the two fluorophores, XL-665 and Eu—K, are available in G. Mathis et al., Anticancer Research, 1997, 17, pages 3011-3014. The energy transfer between the excited europium cryptate to the acceptor XL665 is proportional to the degree of autophosphorylation of IGF-1R. The specific long-lasting signal of XL-665 is measured in a GENios Pro TECAN plate counter. The inhibition of autophosphorylation of IGF-1R at time 30 minutes and 90 minutes with the test compounds of the invention is calculated relative to a 1% in DMSO control, whose activity is measured in the absence of compound. The curve representing the percentage of inhibition as a function of the log of the concentration is established to determine the concentration corresponding to 50% inhibition (IC₅₀),

B) Measurement of the Autophosphorylation of IGF-1R in MCF7 Cells after Stimulation with IGF-1

Cell Culturing and Execution of the Test:

The autophosphorylation of IGF1R in the IGF1-induced cells is evaluated by means of an ELISA technique (Enzyme Linked ImmunoSorbent Assay). The MCF-7 are seeded at 60 000 cells per well in 6-well plates and incubated at 37° C., 5% CO₂ in medium containing 10% serum. After one night in 10% serum, the cells are deprived of serum for 24 hours. The compounds are added to the medium one hour before stimulation with IGF1. After 10 minutes of stimulation with IGF1, the cells are lysed with a buffer (Hepes 50 mM pH 7.6, Triton X100 1%, orthovanadate 2 mM, protease inhibitor cocktail). The cell lysates are incubated on a 96-well plate precoated with an anti-IGF1R antibody, followed by incubation with an anti-phosphotyrosine antibody coupled to the enzyme peroxidase. The level of peroxidase activity (measured by OD with a luminescent substrate) reflects the phosphorylation status of the receptor.

Calculating the Results:

(i) The tests are performed in duplicate and the mean of the two tests is calculated.

(ii) The value of the signal of the maximum response is calculated from the positive control: cells stimulated with IGF1 without compound.

(iii) Value of the signal of the minimum response is calculated from the negative control: cells not stimulated with IGF1 without compound.

(iv) By using these values as maximum (100%) and minimum (0%), respectively, the data were normalized so as to give a percentage of the maximum response.

(v) A curve of dose response is plotted and the IC₅₀ (the concentration of which the compound induces a 50% decrease in the signal) of the compound is calculated by non-linear regression analysis.

### C) Measurement of the Proliferation/Viability of MEF-IGF1R

Cell culture: the MEF-IGF1R cells (stable clone of cells transfected with the receptor hIGF-1R) are cultured at 37° C. under 5% CO2 in EMEM medium containing 10% FCS.

Test procedure: the cells are seeded at 5000 cells per well in 96-well Cytostar plates (Amersham) with 0.2 mL of EMEM culture medium at 37° C. for 18 hours. The cells are then washed twice with EMEM medium and left to culture without serum for 24 hours. The compounds are then added at various concentrations in the presence of rhIGF1 (100 ng/mL) and 0.1 μCi of Thymidine [¹⁴C] (specific activity ˜50 mCi/mmol) to give 0.2 mL of volume per well. After incubation for 72 hours in the presence of the compound, at 37° C. under 5% CO₂, the incorporation of Thymidine [¹⁴C] is measured by counting the radioactivity on a Microbeta trilux counter (Perkin-Elmer). The IC₅₀ is determined from 10 increasing concentrations of the compound.

Calculating the Results:

(i) The tests are performed in duplicate and the mean of the two tests is calculated.

(ii) The value of the signal of the maximum response is calculated from the positive control: cells stimulated with IGF1 without compound.

(iii) Value of the signal of the minimum response is calculated from the negative control: cells not stimulated with IGF1 without compound.

(iv) By using these values as maximum (100%) and minimum (0%), respectively, the data were normalized so as to give a percentage of the maximum response.

(v) A curve of dose response is plotted and the IC₅₀ (the concentration of which the compound induces a 50% decrease in the signal) of the compound is calculated by non-linear regression analysis.

The table below gives the activities of certain examples of the present invention in the three tests A, B and C described above:

Test A*

| Examples | 30′ | 90′ | Test B * | Test C * |
|---|---|---|---|---|
| Example 1 | + | + | + | ++ |
| Example 2 | ++ | ++ | +++ | ++ |
| Example 3 | + | + | + | ++ |
| Example 4 | + | ++ | +++ | ++ |
| Example 5 | ++ | | + | ++ |
| Example 6 | + | | + | +++ |
| Example 7 | + | ++ | + | ++ |
| Example 8 | ++ | +++ | +++ | ++ |
| Example 9 | + | ++ | + | ++ |
| Example 10 | + | + | | + |
| Example 11 | ++ | +++ | +++ | +++ |
| Example 12 | + | + | | + |
| Example 14 | + | + | + | + |
| Example 15 | ++ | ++ | ++ | + |
| Example 16 | ++ | ++ | ++ | + |
| Example 17 | + | ++ | | + |
| Example 18 | ++ | ++ | ++ | + |
| Example 18 | ++ | ++ | ++ | ++ |
| Stage a) | | | | |
| Example 19 | ++ | ++ | +++ | ++ |
| Example 20 | + | + | + | + |
| Example 21 | ++ | ++ | ++ | + |
| Example 22 | ++ | ++ | ++ | ++ |
| Example 23 | ++ | ++ | ++ | ++ |
| Example 25 | + | ++ | | + |
| Example 27 | ++ | ++ | ++ | +++ |
| Example 28 | ++ | ++ | +++ | ++ |
| Example 29 | ++ | ++ | ++ | ++ |
| Example 30 | ++ | +++ | ++ | + |
| Example 31 | + | ++ | | + |
| Example 32 | ++ | ++ | | + |
| Example 33 | ++ | ++ | | + |
| Example 34 | ++ | ++ | ++ | ++ |
| Example 35 | ++ | ++ | ++ | ++ |
| Example 36 | ++ | ++ | ++ | ++ |
| Example 37 | + | ++ | | ++ |
| Example 38 | ++ | ++ | | + |
| Example 39 | ++ | ++ | + | + |
| Example 40 | ++ | +++ | | + |
| Example 40A | ++ | +++ | +++ | ++ |
| Example 40B | ++ | +++ | | |
| Stage a) | | | | |
| Example 40C | + | ++ | ++ | ++ |
| Example 40D | ++ | ++ | ++ | + |
| Example 40E | ++ | ++ | ++ | ++ |
| Example 40F | + | ++ | + | + |
| Example 40G | ++ | ++ | ++ | + |
| Example 40K | | | | |
| Example 40L | ++ | +++ | ++ | + |

*For tests A, B and C, the IC₅₀ (nM) are distributed as follows:
+ > 100 nM
10 nM < ++ < 100 nM
+++ < 10 nM

The examples of pharmaceutical compositions that follow form part of the present invention: it may be noted that pharmaceutical compositions prepared with the other products of formula (I), salts thereof or prodrugs thereof according to the present invention also form part of the present invention.

### EXAMPLE 41

### Pharmaceutical Composition

Tablet corresponding to the following formula were prepared:

Products of Example 1 . . . 0.2 g

Excipient for a finished tablet weighing . . . 1 g

(details of the excipient: lactose, talc, starch, magnesium stearate).

### EXAMPLE 42

### Pharmaceutical Composition

Tablets corresponding to the following formula were prepared:

### Product of Example 9 . . . 0.2 g

Excipient for a finished tablet weighing . . . 1 g
(details of the excipient: lactose, talc, starch, magnesium stearate).

What is claimed is:

1) A compound of formula (I): 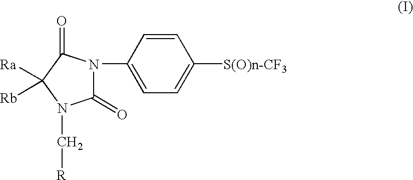
in which:
n represents the integer 0 or 2
Ra and Rb represent CH3 or form, together with the carbon atom to which they are attached, a cycloalkyl radical,
R represents a pyridyl or pyrimidinyl radical substituted with a radical NR1R2,
NR1R2 being such that:
one from among R1 and R2 represents a hydrogen atom or an alkyl radical, and the other from among R1 and R2 is chosen from a hydrogen atom and alkyl radicals optionally substituted with a radical chosen from hydroxyl, alkoxy, aziridyl, azetidinyl, pyrrolidinyl, piperidyl, morpholinyl, and piperazinyl, which is itself optionally substituted on its second nitrogen atom with an alkyl radical; optionally substituted cycloalkyl, heterocycloalkyl, aryl and heteroaryl radicals; and the radical CO—R3 with R3 chosen from NR4R5 and optionally substituted alkoxy, heterocycloalkyl, aryl, aryloxy and heteroaryl radicals;
R4 and R5, which may be identical to or different from R1 and R2, are such that:
either one from among R4 and R5 represents a hydrogen atom or an alkyl radical, and the other from among R4 and R5 is chosen from a hydrogen atom and alkyl radicals optionally substituted with a radical chosen from hydroxyl, alkoxy, aziridyl, azetidinyl, pyrrolidinyl, piperidyl, morpholinyl, and piperazinyl, which is itself optionally substituted on its second nitrogen atom with an alkyl radical; optionally substituted cycloalkyl, heterocycloalkyl, aryl and heteroaryl radicals;
or R4 and R5 form, with the nitrogen atom to which they are attached, a cyclic amine optionally containing another heteroatom chosen from N and O, which is optionally substituted,
all the above aryl, phenyl, aryloxy and heteroaryl radicals, and also the cyclic amine NR4R5, being optionally substituted with one to three radicals, which may be identical or different, chosen from halogen atoms and alkyl, phenyl, NH2, NHAlk, N(Alk)2, CO—NHAlk and CO—N(Alk)2 radicals;
or an addition salt with a mineral or organic acid or with a mineral or organic base of said compound of formula (I);
said compound being in any possible racemic, enantiomeric or diastereoisomeric isomer form.

2) A compound of formula (I) according to claim 1: 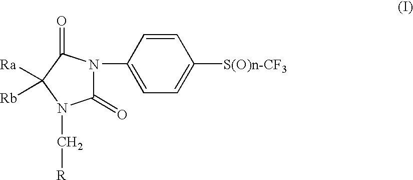
in which:
n represents the integer 0 or 2,
Ra and Rb represent CH3,
R represents a pyridyl or pyrimidinyl radical substituted with
a radical NR1R2,
NR1R2 being such that:
one from among R1 and R2 represents a hydrogen atom or an alkyl radical, and the other from among R1 and R2 is chosen from a hydrogen atom and alkyl radicals optionally substituted with a radical chosen from hydroxyl, alkoxy, aziridyl, azetidinyl, pyrrolidinyl, piperidyl, morpholinyl, and piperazinyl, which is itself optionally substituted on its second nitrogen atom with an alkyl radical; optionally substituted cycloalkyl, heterocycloalkyl, phenyl, pyrimidinyl and pyridyl radicals; and
the radical CO—R3 with R3 chosen from NR4R5 and optionally substituted alkoxy, piperidyl, phenyl and phenoxy radicals;
R4 and R5, which may be identical to or different from R1 and R2, are such that:
either one from among R4 and R5 represents a hydrogen atom or an alkyl radical, and the other from among R4 and R5 is chosen from a hydrogen atom and alkyl radicals optionally substituted with a radical chosen from hydroxyl, alkoxy, aziridyl, azetidinyl, pyrrolidinyl, piperidyl, morpholinyl, and piperazinyl, which is itself optionally substituted on its second nitrogen atom with an alkyl radical; optionally substituted cycloalkyl, heterocycloalkyl, phenyl, pyrimidinyl and pyridyl radicals;
or R4 and R5 form, with the nitrogen atom to which they are attached, a cyclic amine optionally containing another heteroatom chosen from N and O, which is optionally substituted,
all the above phenyl, pyrimidinyl and pyridyl radicals being optionally substituted with one to three radicals, which may be identical or different, chosen from halogen atoms and alkyl, phenyl, NH2, NHAlk, N(Alk)2, CO—NHAlk and CO—N(Alk)2 radicals;
or an addition salt with a mineral or organic acid or with a mineral or organic base of said compound of formula (I);
said compound being in any possible racemic, enantiomeric or diastereoisomeric isomer form.

3) A compound of formula (I) according to claim 1 in which:
n represents the integer 0 or 2
R represents a pyridyl or pyrimidinyl radical substituted with
a radical NR1R2,
NR1R2 being such that R1 represents a hydrogen atom or an alkyl radical, and R2 is chosen from a hydrogen atom and alkyl radicals optionally substituted with a hydroxyl, aziridyl, azetidinyl, pyrrolidinyl, piperidyl, morpholinyl, or piperazinyl, which is itself optionally substituted on its second nitrogen atom with an alkyl radical; 3- to 6-membered cycloalkyl radicals; an optionally substituted phenyl radical;
a pyrimidinyl radical; a pyridyl radical optionally substituted with a halogen atom; and the radical CO—R3 with R3 chosen from NR4R5 and optionally substituted alkoxy, piperidyl and phenyl radicals;
R4 and R5, which may be identical to or different from R1 and R2, are such that:
either one from among R4 and R5 represents a hydrogen atom or an alkyl radical, and the other from among R4 and R5 is chosen from a hydrogen atom and alkyl radicals optionally substituted with a hydroxyl, aziridyl, azetidinyl, pyrrolidinyl, piperidyl, morpholinyl, or piperazinyl, which is itself optionally substituted on its second nitrogen atom with an alkyl radical; 3- to 6-membered cycloalkyl radicals; an optionally substituted phenyl radical; a pyrimidinyl radical; a pyridyl radical optionally substituted with a halogen atom;
or R4 and R5 form, with the nitrogen atom to which they are attached, an aziridyl, azetidinyl, pyrrolidinyl, piperidyl, morpholinyl, or piperazinyl, which is itself optionally substituted on its second nitrogen atom with an alkyl radical, all the phenyl radicals being optionally substituted with one to three radicals, which may be identical or different, chosen from halogen atoms, alkyl radicals and radicals CO—NHAlk and CO—N(Alk)2;
or an addition salt with a mineral or organic acid or with a mineral or organic base of said compound of formula (I);
said compound being in any possible racemic, enantiomeric or diastereoisomeric isomer form.

4) A compound of formula (I) according to claim 1 in which:
n represents the integer 0 or 2
R represents a pyridyl or pyrimidinyl radical substituted with
a radical NR1R2,
NR1R2 being such that R1 represents a hydrogen atom or an alkyl radical containing one or two carbon atoms, and R2 is chosen from alkyl radicals containing 1 to 4 carbon atoms optionally substituted with a hydroxyl radical; an optionally substituted phenyl radical; a pyrimidinyl radical; a pyridyl radical optionally substituted with a halogen atom; and the radical CO—R3 with R3 chosen from piperidyl, optionally substituted phenyl, NH(alk) and N(alk)2; all the phenyl radicals being optionally substituted with one to three radicals, which may be identical or different, chosen from halogen atoms and alkyl radicals and radicals CO—NHAlk and CO—N(Alk)2;
or an addition salt with a mineral or organic acid or with a mineral or organic base of said compound of formula (I);
said compound being in any possible racemic, enantiomeric or diastereoisomeric isomer form.

5) A compound of formula (I) according to claim 1 in which:
n represents the integer 0 or 2
R represents a pyridyl or pyrimidinyl radical substituted with a radical NR1R2
in which R1 represents a hydrogen atom and R2 represents an isopropyl radical substituted with a hydroxyl radical; an optionally substituted phenyl radical; a pyrimidinyl radical; a pyridyl radical optionally substituted with a fluorine atom; or a radical CO—R3 with R3 chosen from piperidyl, optionally substituted phenyl, NHCH3 and N(CH3)2; all the phenyl radicals being optionally substituted with one to three radicals, which may be identical or different, chosen from chlorine and fluorine atoms, methyl radicals and the radical CO—N(CH3)2;
or an addition salt with a mineral or organic acid or with a mineral or organic base of said compound of formula (I);
said compound being in any possible racemic, enantiomeric or diastereoisomeric isomer form.

6) A compound of formula (I) according to claim 1 in which n, Ra, Rb and R are as defined in claim 1,
in which the radicals NR1R2 or NR4R5 or alternatively NR1R2 and NR4R5 are chosen from the following radicals named ex 18 to ex 40: 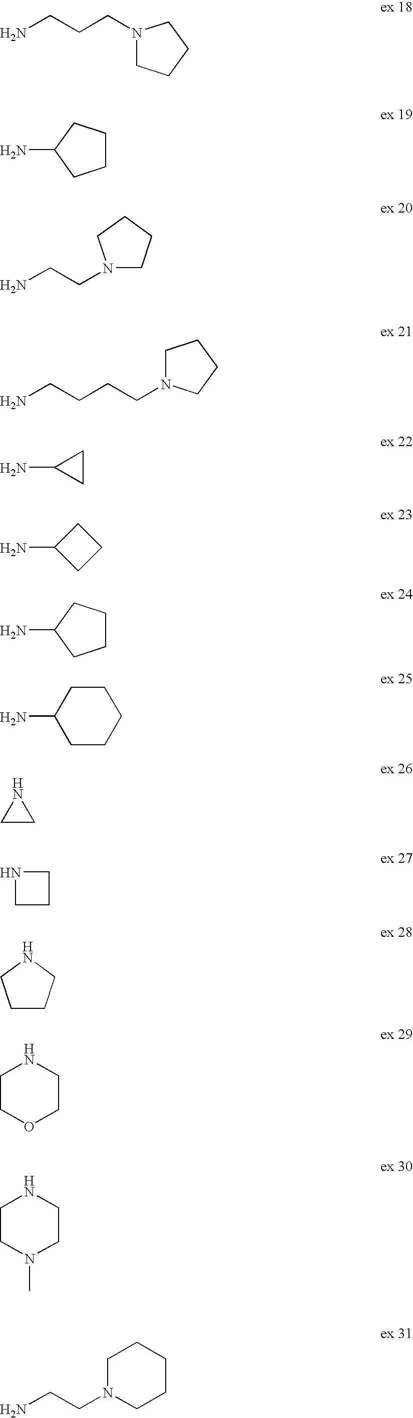 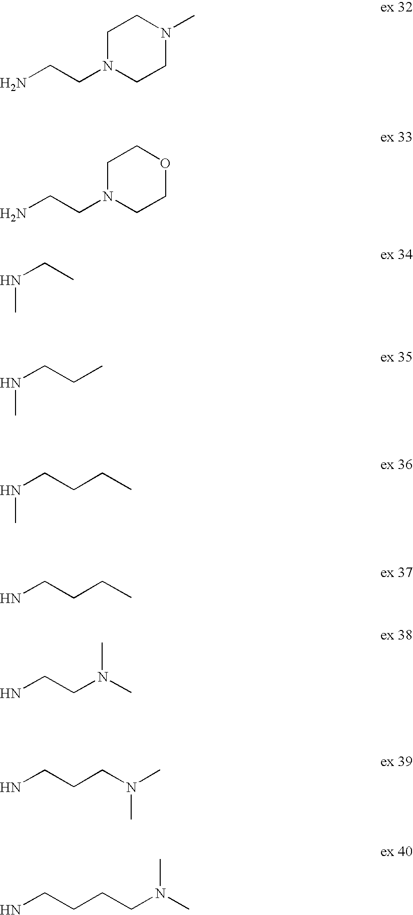
or an addition salt with a mineral or organic acid or with a mineral or organic base of said compound of formula (I);
said compound being in any possible racemic, enantiomeric or diastereoisomeric isomer form.

7) A compound according to claim 6 having the formula (Ia): 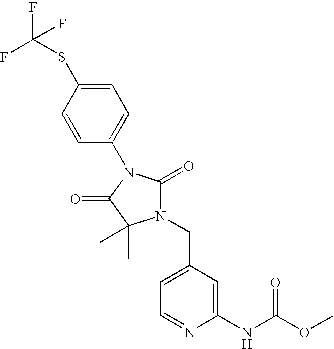
in which n and NR4R5 are as defined in claim 6;
or an addition salt with a mineral or organic acid or with a mineral or organic base of said compound of formula (Ia);
said compound being in any possible racemic, enantiomeric or diastereoisomeric isomer form.

8) A compound of formula (I) according to claim 1, selected from the group consisting of:
1-({2-[(2,5-dichlorophenyl)amino]pyridin-4-yl}methyl)-5,5-dimethyl-3-{4-[(trifluoromethyl)thio]phenyl}imidazolidine-2,4-dione;
N-{4-[(5,5-dimethyl-2,4-dioxo-3-{4-[(trifluoromethyl)thio]-phenyl}imidazolidin-1-yl)methyl]pyridin-2-yl}piperidine-1-carboxamide;
3,4-dichloro-N-{4-[(5,5-dimethyl-2,4-dioxo-3-{4-[(trifluoro-methyl)thio]phenyl}imidazolidin-1-yl)methyl]pyridin-2-yl}benzamide;
1-{4-[(5,5-dimethyl-2,4-dioxo-3-{4-[(trifluoromethyl)-sulfonyl]phenyl}imidazolidin-1-yl)methyl]pyridin-2-yl}-3-methylurea;
1-({2-[(2,5-difluorophenyl)amino]pyridin-4-yl}methyl)-5,5-dimethyl-3-{4-[(trifluoromethyl)thio]phenyl}imidazolidine-2,4-dione;
3,5-dichloro-N-{4-[(5,5-dimethyl-2,4-dioxo-3-{4-[(trifluoro-methyl)thio]phenyl}imidazolidin-1-yl)methyl]pyridin-2-yl}benzamide;
2-chloro-N-{4-[(5,5-dimethyl-2,4-dioxo-3-{4-[(trifluoro-methyl)thio]phenyl}imidazolidin-1-yl)methyl]pyridin-2-yl}-6-fluoro-3-methylbenzamide;
3-({4-[(5,5-dimethyl-2,4-dioxo-3-{4-[(trifluoromethyl)thio]-phenyl}imidazolidin-1-yl)methyl]pyridin-2-yl}amino)-N,N-dimethylbenzamide;
1-[(2-{[(1R)-2-hydroxy-1-methylethyl]amino}pyrimidin-4-yl)-methyl]-5,5-dimethyl-3-{4-[(trifluoromethyl)sulfonyl]phenyl}-imidazolidine-2,4-dione;
3-{4-[(5,5-dimethyl-2,4-dioxo-3-{4-[(trifluoromethyl)thio]-phenyl}imidazolidin-1-yl)methyl]pyrimidin-2-yl}-1,1-dimethyl-urea;
5,5-dimethyl-1-{[2-(pyridin-3-ylamino)pyrimidin-4-yl]methyl}-3-{4-[(trifluoromethyl)thio]phenyl}imidazolidine-2,4-dione;
3-{4-[(5,5-dimethyl-2,4-dioxo-3-{4-[(trifluoromethyl)-sulfonyl]phenyl}imidazolidin-1-yl)methyl]pyrimidin-2-yl}-1,1-dimethylurea;
5,5-dimethyl-1-{[2-(pyrimidin-5-ylamino)pyridin-4-yl]methyl}-3-{4-[(trifluoromethyl)sulfonyl]phenyl}imidazolidine-2,4-dione;
5,5-dimethyl-1-{[2-(pyrimidin-5-ylamino)pyridin-4-yl]methyl}-3-{4-[(trifluoromethyl)thio]phenyl}imidazolidine-2,4-dione;
5,5-dimethyl-1-{[2-(pyrimidin-5-ylamino)pyrimidin-4-yl]-methyl}-3-{4-[(trifluoromethyl)thio]phenyl}imidazolidine-2,4-dione;
5,5-dimethyl-1-{[2-(pyrimidin-5-ylamino)pyrimidin-4-yl]-methyl}-3-{4-[(trifluoromethyl)sulfonyl]phenyl}imidazolidine-2,4-dione; and
1-({2-[(5-fluoropyridin-3-yl)amino]pyridin-4-yl}methyl)-5,5-dimethyl-3-{4-[(trifluoromethyl)thio]phenyl}imidazolidine-2,4-dione;
or an addition salt with a mineral or organic acid or with a mineral or organic base of said compound of formula (I);
said compound being in any possible racemic, enantiomeric or diastereoisomeric isomer form.

9) A pharmaceutical composition comprising a compound according to claim 1, or a pharmaceutically acceptable addition salt thereof, and one or more pharmaceutically acceptable excipients.

10) A pharmaceutical composition comprising a compound according to claim 7, or a pharmaceutically acceptable addition salt thereof, and one or more pharmaceutically acceptable excipients.

11) A pharmaceutical composition comprising a compound according to claim 8, or a pharmaceutically acceptable addition salt thereof, and one or more pharmaceutically acceptable excipients.

12) The pharmaceutical composition according to claim 9, further comprising another chemotherapy medicament for combating cancer.

13) A method for inhibiting the activity of a protein kinase, comprising contacting the protein kinase with a compound according to claim 1, or a pharmaceutically acceptable addition salt thereof.

14) The method according to claim 13, wherein the protein kinase is in a cell culture.

15) The method according to claim 13, wherein the protein kinase is in a mammal.

16) The method according to claim 13, wherein the protein kinase is a protein tyrosine kinase.

17) The method according to claim 13, wherein the protein kinase is IGF1R.

18) A method for the prevention or treatment of a disease characterized by deregulation of the activity of a protein kinase comprising administering to a patient in need of said prevention or treatment a therapeutically effective amount of a compound of formula (I) according to claim 1 or a pharmaceutically acceptable addition salt thereof.

19) The method according to claim 18 wherein the disease is selected from the group consisting of disorders of blood vessel proliferation, fibrotic disorders, disorders of mesangial cell proliferation, metabolic disorders, allergies, asthma, thrombosis, diseases of the nervous system, retinopathy, psoriasis, rheumatoid arthritis, diabetes, muscle degeneration, oncology diseases and cancers.

20) The method according to claim 18, wherein the disease to be treated is cancer.

21) The method according to claim 18, wherein the disease to be treated is a cancer of solid or liquid tumours.

22) The method according to claim 18, wherein the disease to be treated is a cancer that is resistant to cytotoxic agents.

23) The method according to claim 19, wherein the cancer is selected from the group consisting of breast cancer, stomach cancer, cancer of the colon, lung cancer, cancer of the ovaries, cancer of the uterus, brain cancer, cancer of the kidney, cancer of the larynx, cancer of the lymphatic system, cancer of the thyroid, cancer of the urogenital tract, cancer of the tract including the seminal vesicle and prostate, bone cancer, cancer of the pancreas and melanomas.

24) The method according to claim 22, wherein the cancer is selected from the group consisting of breast cancer, cancer of the colon and lung cancer.

25) The method according to claim 22, wherein the compound is administered in combination with a chemotherapy or radiotherapy, or alternatively in combination with other therapeutic agents.

26) The method according to claim 24 wherein the other therapeutic agents are antitumour agents.
